SEQUENCE LISTING (101 sequences)

SEQ ID NO: 1 - protein, 449 aa, Escherichia coli
  Lysine-sensitive aspartokinase 3 (1)..(449)
MSEIVVSKFGGTSVADFDAMNRSADIVLSDANVRLVVLSASAGITNLLVALAEGLEPGERFEKLDAIRNIQFAILERLRYPNVIREEIERLLENITVLAEAAALATSPALTDELVSHGELMSTLLFVEILRERDVQAQWFDVRKVMRTNDRFGRAEPDIAALAELAALQLLPRLNEGLVITQGFIGSENKGRTTTLGRGGSDYTAALLAEALHASRVDIWTDVPGIYTTDPRVVSAAKRIDEIAFAEAAEMATFGAKVLHPATLLPAVRSDIPVFVGSSKDPRAGGTLVCNKTENPPLFRALALRRNQTLLTLHSLNMLHSRGFLAEVFGILARHNISVDLITTSEVSVALTLDTTGSTSTGDTLLTQSLLMELSALCRVEVEEGLALVALIGNDLSKACGVGKEVFGVLEPFNIRMICYGASSHNLCFLVPGEDAEQVVQKLHSNLFE

SEQ ID NO: 2 - protein, 366 aa, Escherichia coli
  Aspartate-semialdehyde dehydrogenase (1)..(366)
MKNVGFIGWRGMVGSVLMQRMVEERDFDAIRPVFFSTSQLGQAAPSFGGTTGTLQDAFDLEALKALDIIVTCQGGDYTNEIYPKLRESGWQGYWIDAASSLRMKDDAIIILDPVNQDVITDGLNNGIRTFVGGNCTVSLMLMSLGGLFANDLVDWVSVATYQAASGGGARHMRELLTQMGHLYGHVADELATPSSAILDIERKVTTLTRSGELPVDNFGVPLAGSLIPWIDKQLDNGQSREEWKGQAETNKILNTSSVIPVDGLCVRVGALRCHSQAFTIKLKKDVSIPTVEELLAAHNPWAKVVPNDREITMRELTPAAVTGTLTTPVGRLRKLNMGPEFLSAFTVGDQLLWGAAEPLRRMLRQL

SEQ ID NO: 3 - protein, 292 aa, Escherichia coli
  4-hydroxy-tetrahydrodipicolinate synthase (1)..(292)
MFTGSIVAIVTPMDEKGNVCRASLKKLIDYHVASGTSAIVSVGTTGESATLNHDEHADVVMMTLDLADGRIPVIAGTGANATAEAISLTQRFNDSGIVGCLTVTPYYNRPSQEGLYQHFKAIAEHTDLPQILYNVPSRTGCDLLPETVGRLAKVKNIIGIKEATGNLTRVNQIKELVSDDFVLLSGDDASALDFMQLGGHGVISVTANVAARDMAQMCKLAAEGHFAEARVINQRLMPLHNKLFVEPNPIPVKWACKELGLVATDTLRLPMTPITDSGRETVRAALKHAGLL

SEQ ID NO: 4 - protein, 397 aa, Corynebacterium glutamicum
MALALVLNSGSSSIKFQLVNPENSAIDEPYVSGLVEQIGEPNGRIVLKIEGEKYTLETPIADHSEGLNLAFDLMDQHNCGPSQLEITAVGHRVVHGGILFSAPELITDEIVEMIRDLIPLAPLHNPANVDGIDVARKILPDVPHVAVFDTGFFHSLPPAAALYAINKDVAAEHGIRRYGFHGTSHEFVSKRVVEILEKPTEDINTITFHLGNGASMAAVQGGRAVDTSMGMTPLAGLVMGTRSGDIDPGIVFHLSRTAGMSIDEIDNLLNKKSGVKGLSGVNDFRELREMIDNNDQDAWSAYNIYIHQLRRYLGSYMVALGRVDTIVFTAGVGENAQFVREDALAGLEMYGIEIDPERNALPNDGPRLISTDASKVKVFVIPTNEELAIARYAVKFA

SEQ ID NO: 5 - protein, 273 aa, Escherichia coli
  4-hydroxy-tetrahydrodipicolinate reductase (1)..(273)
MHDANIRVAIAGAGGRMGRQLIQAALALEGVQLGAALEREGSSLLGSDAGELAGAGKTGVTVQSSLDAVKDDFDVFIDFTRPEGTLNHLAFCRQHGKGMVIGTTGFDEAGKQAIRDAAADIAIVFAANFSVGVNVMLKLLEKAAKVMGDYTDIEIIEAHHRHKVDAPSGTALAMGEAIAHALDKDLKDCAVYSREGHTGERVPGTIGFATVRAGDIVGEHTAMFADIGERLEITHKASSRMTFANGAVRSALWLSGKESGLFDMRDVLDLNNL

SEQ ID NO: 6 - protein, 420 aa, Escherichia coli
  Diaminopimelate decarboxylase (1)..(420)
MPHSLFSTDTDLTAENLLRLPAEFGCPVWVYDAQIIRRQIAALKQFDVVRFAQKACSNIHILRLMREQGVKVDSVSLGEIERALAAGYNPQTHPDDIVFTADVIDQATLERVSELQIPVNAGSVDMLDQLGQVSPGHRVWLRVNPGFGHGHSQKTNTGGENSKHGIWYTDLPAALDVIQRHHLQLVGIHMHIGSGVDYAHLEQVCGAMVRQVIEFGQDLQAISAGGGLSVPYQQGEEAVDTEHYYGLWNAAREQIARHLGHPVKLEIEPGRFLVAQSGVLITQVRSVKQMGSRHFVLVDAGFNDLMRPAMYGSYHHISALAADGRSLEHAPTVETVVAGPLCESGDVFTQQEGGNVETRALPEVKAGDYLVLHDTGAYGASMSSNYNSRPLLPEVLFDNGQARLIRRRQTIEELLALELL

SEQ ID NO: 7 - protein, 299 aa, Escherichia coli
  Lysine exporter LysO (1)..(299)
MFSGLLIILVPLIVGYLIPLRQQAALKVINQLLSWMVYLILFFMGISLAFLDNLASNLLAILHYSAVSITVILLCNIAALMWLERGLPWRNHHQQEKLPSRIAMALESLKLCGVVVIGFAIGLSGLAFLQHATEASEYTLILLLFLVGIQLRNNGMTLKQIVLNRRGMIVAVVVVVSSLIGGLINAFILDLPINTALAMASGFGWYSLSGILLTESFGPVIGSAAFFNDLARELIAIMLIPGLIRRSRSTALGLCGATSMDFTLPVLQRTGGLDMVPAAIVHGFILSLLVPILIAFFSA

SEQ ID NO: 8 - protein, 223 aa, Escherichia coli
  Uncharacterized membrane protein YahN (1)..(223)
MMQLVHLFMDEITMDPLHAVYLTVGLFVITFFNPGANLFVVVQTSLASGRRAGVLTGLGVALGDAFYSGLGLFGLATLITQCEEIFSLIRIVGGAYLLWFAWCSMRRQSTPQMSTLQQPISAPWYVFFRRGLITDLSNPQTVLFFISIFSVTLNAETPTWARLMAWAGIVLASIIWRVFLSQAFSLPAVRRAYGRMQRVASRVIGAIIGVFALRLIYEGVTQR

SEQ ID NO: 9 - protein, 361 aa, Corynebacterium glutamicum
  Lysine exporter protein (1)..(361)
MAVMAYQPADNRYDDMIYRRVGNSGLKLPAISLGLWHNFGDDKPLSTQRSIIHRAFDRGVTHFDLANNYGPPAGSAETNFGRILREDLKSHRDELIISSKAGWDMWPGPYGFGGSRKYLVSSLDQSLTRLGLDYVDIFYHHRPDPDTPLEETMYALRDIVASGKALYVGISSYGPELTAEAAEFMAEEGCPLLIHQPSYSIINRWVEEPGDDGENLLQSAANNGLGVIAFSPLAQGLLTDKYLDGIPEGSRASQGKSLSEGMLNVNNIDMVRKLNDIAQERGQSLAQMALAWVLREQGEYGADTVTSALIGASSVEQLDNSLDSLNNLEFSDAELEAIDEISHDAGINIWAKATDSKTREN

SEQ ID NO: 10 - protein, 883 aa, Escherichia coli
  Phosphoenolpyruvate carboxylase (1)..(883)
MNEQYSALRSNVSMLGKVLGETIKDALGEHILERVETIRKLSKSSRAGNDANRQELLTTLQNLSNDELLPVARAFSQFLNLANTAEQYHSISPKGEAASNPEVIARTLRKLKNQPELSEDTIKKAVESLSLELVLTAHPTEITRRTLIHKMVEVNACLKQLDNKDIADYEHNQLMRRLRQLIAQSWHTDEIRKLRPSPVDEAKWGFAVVENSLWQGVPNYLRELNEQLEENLGYKLPVEFVPVRFTSWMGGDRDGNPNVTADITRHVLLLSRWKATDLFLKDIQVLVSELSMVEATPELLALVGEEGAAEPYRYLMKNLRSRLMATQAWLEARLKGEELPKPEGLLTQNEELWEPLYACYQSLQACGMGIIANGDLLDTLRRVKCFGVPLVRIDIRQESTRHTEALGELTRYLGIGDYESWSEADKQAFLIRELNSKRPLLPRNWQPSAETREVLDTCQVIAEAPQGSIAAYVISMAKTPSDVLAVHLLLKEAGIGFAMPVAPLFETLDDLNNANDVMTQLLNIDWYRGLIQGKQMVMIGYSDSAKDAGVMAASWAQYQAQDALIKTCEKAGIELTLFHGRGGSIGRGGAPAHAALLSQPPGSLKGGLRVTEQGEMIRFKYGLPEITVSSLSLYTGAILEANLLPPPEPKESWRRIMDELSVISCDVYRGYVRENKDFVPYFRSATPEQELGKLPLGSRPAKRRPTGGVESLRAIPWIFAWTQNRLMLPAWLGAGTALQKVVEDGKQSELEAMCRDWPFFSTRLGMLEMVFAKADLWLAEYYDQRLVDKALWPLGKELRNLQEEDIKVVLAIANDSHLMADLPWIAESIQLRNIYTDPLNVLQAELLHRSRQAEKEGQEPDPRVEQALMVTIAGIAAGMRNTG

SEQ ID NO: 11 - protein, 510 aa, Escherichia coli
  proton-translocating transhydrogenase (1)..(510)
  proton-translocating transhydrogenase (pntA) (1)..(510)
MRIGIPRERLTNETRVAATPKTVEQLLKLGFTVAVESGAGQLASFDDKAFVQAGAEIVEGNSVWQSEIILKVNAPLDDEIALLNPGTTLVSFIWPAQNPELMQKLAERNVTVMAMDSVPRISRAQSLDALSSMANIAGYRAIVEAAHEFGRFFTGQITAAGKVPPAKVMVIGAGVAGLAAIGAANSLGAIVRAFDTRPEVKEQVQSMGAEFLELDFKEEAGSGDGYAKVMSDAFIKAEMELFAAQAKEVDIIVTTALIPGKPAPKLITREMVDSMKAGSVIVDLAAQNGGNCEYTVPGEIFTTENGVKVIGYTDLPGRLPTQSSQLYGTNLVNLLKLLCKEKDGNITVDFDDVVIRGVTVIRAGEITWPAPPIQVSAQPQAAQKAAPEVKTEEKCTCSPWRKYALMALAIILFGWMASVAPKEFLGHFTVFALACVVGYYVVWNVSHALHTPLMSVTNAISGIIVVGALLQIGQGGWVSFLSFIAVLIASINIFGGFTVTQRMLKMFRKN

SEQ ID NO: 12 - protein, 1140 aa, Corynebacterium glutamicum
  Pyruvate carboxylase (1)..(1140)
MSTHTSSTLPAFKKILVANRGEIAVRAFRAALETGAATVAIYPREDRGSFHRSFASEAVRIGTEGSPVKAYLDIDEIIGAAKKVKADAIYPGYGFLSENAQLARECAENGITFIGPTPEVLDLTGDKSRAVTAAKKAGLPVLAESTPSKNIDEIVKSAEGQTYPIFVKAVAGGGGRGMRFVASPDELRKLATEASREAEAAFGDGAVYVERAVINPQHIEVQILGDHTGEVVHLYERDCSLQRRHQKVVEIAPAQHLDPELRDRICADAVKFCRSIGYQGAGTVEFLVDEKGNHVFIEMNPRIQVEHTVTEEVTEVDLVKAQMRLAAGATLKELGLTQDKIKTHGAALQCRITTEDPNNGFRPDTGTITAYRSPGGAGVRLDGAAQLGGEITAHFDSMLVKMTCRGSDFETAVARAQRALAEFTVSGVATNIGFLRALLREEDFTSKRIATGFIADHPHLLQAPPADDEQGRILDYLADVTVNKPHGVRPKDVAAPIDKLPNIKDLPLPRGSRDRLKQLGPAAFARDLREQDALAVTDTTFRDAHQSLLATRVRSFALKPAAEAVAKLTPELLSVEAWGGATYDVAMRFLFEDPWDRLDELREAMPNVNIQMLLRGRNTVGYTPYPDSVCRAFVKEAASSGVDIFRIFDALNDVSQMRPAIDAVLETNTAVAEVAMAYSGDLSDPNEKLYTLDYYLKMAEEIVKSGAHILAIKDMAGLLRPAAVTKLVTALRREFDLPVHVHTHDTAGGQLATYFAAAQAGADAVDGASAPLSGTTSQPSLSAIVAAFAHTRRDTGLSLEAVSDLEPYWEAVRGLYLPFESGTPGPTGRVYRHEIPGGQLSNLRAQATALGLADRFELIEDNYAAVNEMLGRPTKVTPSSKVVGDLALHLVGAGVDPADFAADPQKYDIPDSVIAFLRGELGNPPGGWPEPLRTRALEGRSEGKAPLTEVPEEEQAHLDADDSKERRNSLNRLLFPKPTEEFLEHRRRFGNTSALDDREFFYGLVEGRETLIRLPDVRTPLLVRLDAISEPDDKGMRNVVANVNGQIRPMRVRDRSVESVTATAEKADSSNKGHVAAPFAGVVTVTVAEGDEVKAGDAVAIIEAMKMEATITASVDGKIDRVVVPAATKVEGGDLIVVVS

SEQ ID NO: 13 - protein, 331 aa, Escherichia coli
  glyceraldehyde-3-phosphate dehydrogenase (NADP-dependent) (1)..(331)
MTIKVGINGFGRIGRIVFRAAQKRSDIEIVAINDLLDADYMAYMLKYDSTHGRFDGTVEVKDGHLIVNGKKIRVTAERDPANLKWDEVGVDVVAEATGLFLTDETARKHITAGAKKVVMTGPSKDNTPMFVKGANFDKYAGQDIVSNASCTTNCLAPLAKVINDNFGIIEGLMTTVHATTATQKTVDGPSHKDWRGGRGASQNIIPSSTGAAKAVGKVLPELNGKLTGMAFRVPTPNVSVVDLTVRLEKAATYEQIKAAVKAAAEGEMKGVLGYTEDDVVSTDFNGEVCTSVFDAKAGIALNDNFVKLVSWYDNETGYSNKVLDLIAHISK

SEQ ID NO: 14 - protein, 445 aa, Corynebacterium glutamicum
  Homoserine dehydrogenase (1)..(445)
MTSASAPSFNPGKGPGSAVGIALLGFGTVGTEVMRLMTEYGDELAHRIGGPLEVRGIAVSDISKPREGVAPELLTEDAFALIEREDVDIVVEVIGGIEYPREVVLAALKAGKSVVTANKALVAAHSAELADAAEAANVDLYFEAAVAGAIPVVGPLRRSLAGDQIQSVMGIVNGTTNFILDAMDSTGADYADSLAEATRLGYAEADPTADVEGHDAASKAAILASIAFHTRVTADDVYCEGISNISAADIEAAQQAGHTIKLLAICEKFTNKEGKSAISARVHPTLLPVSHPLASVNKSFNAIFVEAEAAGRLMFYGNGAGGAPTASAVLGDVVGAARNKVHGGRAPGESTYANLPIADFGETTTRYHLDMDVEDRVGVLAELASLFSEQGISLRTIRQEERDDDARLIVVTHSALESDLSRTVELLKAKPVVKAINSVIRLERD

SEQ ID NO: 15 - protein, 310 aa, Escherichia coli
  Homoserine kinase (1)..(310)
MVKVYAPASSANMSVGFDVLGAAVTPVDGALLGDVVTVEAAETFSLNNLGRFADKLPSEPRENIVYQCWERFCQELGKQIPVAMTLEKNMPIGSGLGSSACSVVAALMAMNEHCGKPLNDTRLLALMGELEGRISGSIHYDNVAPCFLGGMQLMIEENDIISQQVPGFDEWLWVLAYPGIKVSTAEARAILPAQYRRQDCIAHGRHLAGFIHACYSRQPELAAKLMKDVIAEPYRERLLPGFRQARQAVAEIGAVASGISGSGPTLFALCDKPETAQRVADWLGKNYLQNQEGFVHICRLDTAGARVLEN

SEQ ID NO: 16 - protein, 377 aa, Corynebacterium glutamicum
  Homoserin O-Acetyltransferase (1)..(377)
MPTLAPSGQLEIQAIGDVSTEAGAIITNAEIAYHRWGEYRVDKEGRSNVVLIEHALTGDSNAADWWADLLGPGKAINTDIYCVICTNVIGGCNGSTGPGSMHPDGNFWGNRFPATSIRDQVNAEKQFLDALGITTVAAVLGGSMGGARTLEWAAMYPETVGAAAVLAVSARASAWQIGIQSAQIKAIENDHHWHEGNYYESGCNPATGLGAARRIAHLTYRGELEIDERFGTKAQKNENPLGPYRKPDQRFAVESYLDYQADKLVQRFDAGSYVLLTDALNRHDIGRDRGGLNKALESIKVPVLVAGVDTDILYPYHQQEHLSRNLGNLLAMAKIVSPVGHDAFLTESRQMDRIVRNFFSLISPDEDNPSTYIEFYI

SEQ ID NO: 17 - protein, 502 aa, Corynebacterium glutamicum
MSDRIASEKLRSKLMSADEAAQFVNHGDKVGFSGFTGAGYPKALPTAIANRAKEAHGAGNDYAIDLFTGASTAPDCDGVLAEADAIRWRMPYASDPIMRNKINSGSMGYSDIHLSHSGQQVEEGFFGQLNVAVIEITRITEEGYIIPSSSVGNNVEWLNAAEKVILEVNSWQSEDLEGMHDIWSVPALPNRIAVPINKPGDRIGKTYIEFDTDKVVAVVETNTADRNAPFKPVDDISKKIAGNFLDFLESEVAAGRLSYDGYIMQSGVGNVPNAVMAGLLESKFENIQAYTEVIQDGMVDLIDAGKMTVASATSFSLSPEYAEKMNNEAKRYRESIILRPQQISNHPEVIRRVGLIATNGLIEADIYGNVNSTNVSGSRVMNGIGGSGDFTRNGYISSFITPSEAKGGAISAIVPFASHIDHTEHDVMVVISEYGYADLRGLAPRERVAKMIGLAHPDYRPLLEEYYARATSGDNKYMQTPHDLATAFDFHINLAKNGSMKA

SEQ ID NO: 18 - protein, 428 aa, Escherichia coli
  threonine synthase (1)..(428)
MKLYNLKDHNEQVSFAQAVTQGLGKNQGLFFPHDLPEFSLTEIDEMLKLDFVTRSAKILSAFIGDEIPQEILEERVRAAFAFPAPVANVESDVGCLELFHGPTLAFKDFGGRFMAQMLTHIAGDKPVTILTATSGDTGAAVAHAFYGLPNVKVVILYPRGKISPLQEKLFCTLGGNIETVAIDGDFDACQALVKQAFDDEELKVALGLNSANSINISRLLAQICYYFEAVAQLPQETRNQLVVSVPSGNFGDLTAGLLAKSLGLPVKRFIAATNVNDTVPRFLHDGQWSPKATQATLSNAMDVSQPNNWPRVEELFRRKIWQLKELGYAAVDDETTQQTMRELKELGYTSEPHAAVAYRALRDQLNPGEYGLFLGTAHPAKFKESVEAILGETLDLPKELAERADLPLLSHNLPADFAALRKLMMNHQ

SEQ ID NO: 19 - protein, 206 aa, Escherichia coli
  threonine exporter (1)..(206)
MLMLFLTVAMVHIVALMSPGPDFFFVSQTAVSRSRKEAMMGVLGITCGVMVWAGIALLGLHLIIEKMAWLHTLIMVGGGLYLCWMGYQMLRGALKKEAVSAPAPQVELAKSGRSFLKGLLTNLANPKAIIYFGSVFSLFVGDNVGTTARWGIFALIIVETLAWFTVVASLFALPQMRRGYQRLAKWIDGFAGALFAGFGIHLIISR

SEQ ID NO: 20 - protein, 462 aa, Escherichia coli
  proton-translocating transhydrogenase (pntB) (1)..(462)
MSGGLVTAAYIVAAILFIFSLAGLSKHETSRQGNNFGIAGMAIALIATIFGPDTGNVGWILLAMVIGGAIGIRLAKKVEMTEMPELVAILHSFVGLAAVLVGFNSYLHHDAGMAPILVNIHLTEVFLGIFIGAVTFTGSVVAFGKLCGKISSKPLMLPNRHKMNLAALVVSFLLLIVFVRTDSVGLQVLALLIMTAIALVFGWHLVASIGGADMPVVVSMLNSYSGWAAAAAGFMLSNDLLIVTGALVGSSGAILSYIMCKAMNRSFISVIAGGFGTDGSSTGDDQEVGEHREITAEETAELLKNSHSVIITPGYGMAVAQAQYPVAEITEKLRARGINVRFGIHPVAGRLPGHMNVLLAEAKVPYDIVLEMDEINDDFADTDTVLVIGANDTVNPAAQDDPKSPIAGMPVLEVWKAQNVIVFKRSMNTGYAGVQNPLFFKENTHMLFGDAKASVDAILKAL

SEQ ID NO: 21 - protein, 719 aa, Escherichia coli
  acyl-ACP synthetase (1)..(719)
MLFSFFRNLCRVLYRVRVTGDTQALKGERVLITPNHVSFIDGILLGLFLPVRPVFAVYTSISQQWYMRWLKSFIDFVPLDPTQPMAIKHLVRLVEQGRPVVIFPEGRITTTGSLMKIYDGAGFVAAKSGATVIPVRIEGAELTHFSRLKGLVKRRLFPQITLHILPPTQVAMPDAPRARDRRKIAGEMLHQIMMEARMAVRPRETLYESLLSAMYRFGAGKKCVEDVNFTPDSYRKLLTKTLFVGRILEKYSVEGERIGLMLPNAGISAAVIFGAIARRRMPAMMNYTAGVKGLTSAITAAEIKTIFTSRQFLDKGKLWHLPEQLTQVRWVYLEDLKADVTTADKVWIFAHLLMPRLAQVKQQPEEEALILFTSGSEGHPKGVVHSHKSILANVEQIKTIADFTTNDRFMSALPLFHSFGLTVGLFTPLLTGAEVFLYPSPLHYRIVPELVYDRSCTVLFGTSTFLGHYARFANPYDFYRLRYVVAGAEKLQESTKQLWQDKFGLRILEGYGVTECAPVVSINVPMAAKPGTVGRILPGMDARLLSVPGIEEGGRLQLKGPNIMNGYLRVEKPGVLEVPTAENVRGEMERGWYDTGDIVRFDEQGFVQIQGRAKRFAKIAGEMVSLEMVEQLALGVSPDKVHATAIKSDASKGEALVLFTTDNELTRDKLQQYAREHGVPELAVPRDIRYLKQMPLLGSGKPDFVTLKSWVDEAEQHDE

SEQ ID NO: 22 - protein, 561 aa, Escherichia coli
  acyl-CoA synthetase (1)..(561)
MKKVWLNRYPADVPTEINPDRYQSLVDMFEQSVARYADQPAFVNMGEVMTFRKLEERSRAFAAYLQQGLGLKKGDRVALMMPNLLQYPVALFGILRAGMIVVNVNPLYTPRELEHQLNDSGASAIVIVSNFAHTLEKVVDKTAVQHVILTRMGDQLSTAKGTVVNFVVKYIKRLVPKYHLPDAISFRSALHNGYRMQYVKPELVPEDLAFLQYTGGTTGVAKGAMLTHRNMLANLEQVNATYGPLLHPGKELVVTALPLYHIFALTINCLLFIELGGQNLLITNPRDIPGLVKELAKYPFTAITGVNTLFNALLNNKEFQQLDFSSLHLSAGGGMPVQQVVAERWVKLTGQYLLEGYGLTECAPLVSVNPYDIDYHSGSIGLPVPSTEAKLVDDDDNEVPPGQPGELCVKGPQVMLGYWQRPDATDEIIKNGWLHTGDIAVMDEEGFLRIVDRKKDMILVSGFNVYPNEIEDVVMQHPGVQEVAAVGVPSGSSGEAVKIFVVKKDPSLTEESLVTFCRRQLTGYKVPKLVEFRDELPKSNVGKILRRELRDEARGKVDNKA

SEQ ID NO: 23 - protein, 400 aa, Escherichia coli
  fatty acyl kinase (1)..(400)
MSSKLVLVLNCGSSSLKFAIIDAVNGEEYLSGLAECFHLPEARIKWKMDGNKQEAALGAGAAHSEALNFIVNTILAQKPELSAQLTAIGHRIVHGGEKYTSSVVIDESVIQGIKDAASFAPLHNPAHLIGIEEALKSFPQLKDKNVAVFDTAFHQTMPEESYLYALPYNLYKEHGIRRYGAHGTSHFYVTQEAAKMLNKPVEELNIITCHLGNGGSVSAIRNGKCVDTSMGLTPLEGLVMGTRSGDIDPAIIFHLHDTLGMSVDAINKLLTKESGLLGLTEVTSDCRYVEDNYATKEDAKRAMDVYCHRLAKYIGAYTALMDGRLDAVVFTGGIGENAAMVRELSLGKLGVLGFEVDHERNLAARFGKSGFINKEGTRPAVVIPTNEELVIAQDASRLTA

SEQ ID NO: 24 - protein, 301 aa, Clostridium acetobutylicum
  phosphotransacylase (1)..(301)
MIKSFNEIIMKVKSKEMKKVAVAVAQDEPVLEAVRDAKKNGIADAILVGDHDEIVSIALKIGMDVNDFEIVNEPNVKKAALKAVELVSTGKADMVMKGLVNTATFLRSVLNKEVGLRTGKTMSHVAVFETEKFDRLLFLTDVAFNTYPELKEKIDIVNNSVKVAHAIGIENPKVAPICAVEVINPKMPSTLDAAMLSKMSDRGQIKGCVVDGPLALDIALSEEAAHHKGVTGEVAGKADIFLMPNIETGNVMYKTLTYTTDSKNGGILVGTSAPVVLTSRADSHETKMNSIALAALVAGNK

SEQ ID NO: 25 - protein, 355 aa, Clostridium acetobutylicum
  butyrate kinase (1)..(355)
MYRLLIINPGSTSTKIGIYDDEKEIFEKTLRHSAEEIEKYNTIFDQFQFRKNVILDALKEANIEVSSLNAVVGRGGLLKPIVSGTYAVNQKMLEDLKVGVQGQHASNLGGIIANEIAKEINVPAYIVDPVVVDELDEVSRISGMADIPRKSIFHALNQKAVARRYAKEVGKKYEDLNLIVVHMGGGTSVGTHKDGRVIEVNNTLDGEGPFSPERSGGVPIGDLVRLCFSNKYTYEEVMKKINGKGGVVSYLNTIDFKAVVDKALEGDKKCALIYEAFTFQVAKEIGKCSTVLKGNVDAIILTGGIAYNEHVCNAIEDRVKFIAPVVRYGGEDELLALAEGGLRVLRGEEKAKEYK

SEQ ID NO: 26 - protein, 400 aa, Escherichia coli; identical to SEQ ID NO: 23
  propionate kinase (1)..(400)

SEQ ID NO: 27 - protein, 865 aa, Escherichia coli
MLEEYRKHVAERAAEGIAPKPLDANQMAALVELLKNPPAGEEEFLLDLLTNRVPPGVDEAAYVKAGFLAAIAKGEAKSPLLTPEKAIELLGTMQGGYNIHPLIDALDDAKLAPIAAKALSHTLLMFDNFYDVEEKAKAGNEYAKQVMQSWADAEWFLNRPALAEKLTVTVFKVTGETNTDDLSPAPDAWSRPDIPLHALAMLKNAREGIEPDQPGVVGPIKQIEALQQKGFPLAYVGDVVGTGSSRKSATNSVLWFMGDDIPHVPNKRGGGLCLGGKIAPIFFNTMEDAGALPIEVDVSNLNMGDVIDVYPYKGEVRNHETGELLATFELKTDVLIDEVRAGGRIPLIIGRGLTTKAREALGLPHSDVFRQAKDVAESDRGFSLAQKMVGRACGVKGIRPGAYCEPKMTSVGSQDTTGPMTRDELKDLACLGFSADLVMQSFCHTAAYPKPVDVNTHHTLPDFIMNRGGVSLRPGDGVIHSWLNRMLLPDTVGTGGDSHTRFPIGISFPAGSGLVAFAAATGVMPLDMPESVLVRFKGKMQPGITLRDLVHAIPLYAIKQGLLTVEKKGKKNIFSGRILEIEGLPDLKVEQAFELTDASAERSAAGCTIKLNKEPIIEYLNSNIVLLKWMIAEGYGDRRTLERRIQGMEKWLANPELLEADADAEYAAVIDIDLADIKEPILCAPNDPDDARPLSAVQGEKIDEVFIGSCMTNIGHFRAAGKLLDAHKGQLPTRLWVAPPTRMDAAQLTEEGYYSVFGKSGARIEIPGCSLCMGNQARVADGATVVSTSTRNFPNRLGTGANVFLASAELAAVAALIGKLPTPEEYQTYVAQVDKTAVDTYRYLNFNQLSQYTEKADGVIFQTAV

SEQ ID NO: 28 - protein, 305 aa, Corynebacterium glutamicum
MNLFSNGVDVGRRRQAFKAALAAPHIARLPGAFSPLIARSIEEAGFEGVYVSGAVIAADLALPDIGLTTLTEVAHRARQIARVTDLGVLVDADTGFGEPMSAARTVAELEDAGVAGCHLEDQVNPKRCGHLDGKEVVRTDVMVRRIAAAVSARRDPNFVICARTDAAGVEGIDAAIERAKAYLDAGADMIFTEALHSEADFRYFRHAIPDALLLANMTEFGKTTLLSADVLEEIGYNAVIYPVTTLRIAMGQVEQALAEIKEHGTQEGWLDRMQHRSRLYELLRYEDYNVFDQHIFTYRKGENNE

SEQ ID NO: 29 - protein, 865 aa, Escherichia coli; identical to SEQ ID NO: 27

SEQ ID NO: 30 - protein, 389 aa, Escherichia coli
MSDTTILQNSTHVIKPKKSVALSGVPAGNTALCTVGKSGNDLHYRGYDILDLAKHCEFEEVAHLLIHGKLPTRDELAAYKTKLKALRGLPANVRTVLEALPAASHPMDVMRTGVSALGCTLPEKEGHTVSGARDIADKLLASLSSILLYWYHYSHNGERIQPETDDDSIGGHFLHLLHGEKPSQSWEKAMHISLVLYAEHEFNASTFTSRVIAGTGSDMYSAIIGAIGALRGPKHGGANEVSLEIQQRYETPDEAEADIRKRVENKEVVIGFGHPVYTIADPRHQVIKRVAKQLSQEGGSLKMYNIADRLETVMWESKKMFPNLDWFSAVSYNMMGVPTEMFTPLFVIARVTGWAAHIIEQRQDNKIIRPSANYVGPEDRPFVALDKRQ

SEQ ID NO: 31 - protein, 461 aa, Corynebacterium glutamicum
MSDTPTSALITTVNRSFDGFDLEEVAADLGVRLTYLPDEELEVSKVLAADLLAEGPALIIGVGNTFFDAQVAAALGVPVLLLVDKQGKHVALARTQVNNAGAVVAAAFTAEQEPMPDKLRKAVRNHSNLEPVMSAELFENWLLKRARAEHSHIVLPEGDDDRILMAAHQLLDQDICDITILGDPVKIKERATELGLHLNTAYLVNPLTDPRLEEFAEQFAELRKSKSVTIDEAREIMKDISYFGTMMVHNGDADGMVSGAANTTAHTIKPSFQIIKTVPEASVVSSIFLMVLRGRLWAFGDCAVNPNPTAEQLGEIAVVSAKTAAQFGIDPRVAILSYSTGNSGGGSDVDRAIDALAEARRLNPELCVDGPLQFDAAVDPGVARKKMPDSDVAGQANVFIFPDLEAGNIGYKTAQRTGHALAVGPILQGLNKPVNDLSRGATVPDIVNTVAITAIQAGGRS

SEQ ID NO: 32 - protein, 628 aa, Escherichia coli [sequence omitted]

SEQ ID NO: 33 - DNA, 1350 bp, Escherichia coli
atgtctgaaattgttgtctccaaatttggcggtaccagcgtagctgattttgacgccatgaaccgcagcgctgatattgtgctttctgatgccaacgtgcgtttagttgtcctctcggcttctgctggtatcactaatctgctggtcgctttagctgaaggactggaacctggcgagcgattcgaaaaactcgacgctatccgcaacatccagtttgccattctggaacgtctgcgttacccgaacgttatccgtgaagagattgaacgtctgctggagaacattactgttctggcagaagcggcggcgctggcaacgtctccggcgctgacagatgagctggtcagccacggcgagctgatgtcgaccctgctgtttgttgagatcctgcgcgaacgcgatgttcaggcacagtggtttgatgtacgtaaagtgatgcgtaccaacgaccgatttggtcgtgcagagccagatatagccgcgctggcggaactggccgcgctgcagctgctcccacgtctcaatgaaggcttagtgatcacccagggatttatcggtagcgaaaataaaggtcgtacaacgacgcttggccgtggaggcagcgattatacggcagccttgctggcggaggctttacacgcatctcgtgttgatatctggaccgacgtcccgggcatctacaccaccgatccacgcgtagtttccgcagcaaaacgcattgatgaaatcgcgtttgccgaagcggcagagatggcaacttttggtgcaaaagtactgcatccggcaacgttgctacccgcagtacgcagcgatatcccggtctttgtcggctccagcaaagacccacgcgcaggtggtacgctggtgtgcaataaaactgaaaatccgccgctgttccgcgctctggcgcttcgtcgcaatcagactctgctcactttgcacagcctgaatatgctgcattctcgcggtttcctcgcggaagttttcggcatcctcgcgcggcataatatttcggtagacttaatcaccacgtcagaagtgagcgtggcattaatccttgataccaccggttcaacctccactggcgatacgttgctgacgcaatctctgctgatggagctttccgcactgtgtcgggtggaggtggaagaaggtctggcgctggtcgcgttgattggcaatgacctgtcaaaagcctgcggcgttggcaaagaggtattcggcgtactggaaccgttcaacattcgcatgatttgttatggcgcatccagccataacctgtgcttcctggtgcccggcgaagatgccgagcaggtggtgcaaaaactgcatagtaatttgtttgagtaa

SEQ ID NO: 34 - DNA, 2466 bp, Escherichia coli
atgcgagtgttgaagttcggcggtacatcagtggcaaatgcagaacgttttctgcgtgttgccgatattctggaaagcaatgccaggcaggggcaggtggccaccgtcctctctgcccccgccaaaatcaccaaccacctggtggcgatgattgaaaaaaccattagcggccaggatgctttacccaatatcagcgatgccgaacgtatttttgccgaacttttgacgggactcgccgccgcccagccggggttcccgctggcgcaattgaaaactttcgtcgatcaggaatttgcccaaataaaacatgtcctgcatggcattagtttgttggggcagtgcccggatagcatcaacgctgcgctgatttgccgtggcgagaaaatgtcgatcgccattatggccggcgtattagaagcgcgcggtcacaacgttactgttatcgatccggtcgaaaaactgctggcagtggggcattacctcgaatctaccgtcgatattgctgagtccacccgccgtattgcggcaagccgcattccggctgatcacatggtgctgatggcaggtttcaccgccggtaatgaaaaaggcgaactggtggtgcttggacgcaacggttccgactactctgctgcggtgctggctgcctgtttacgcgccgattgttgcgagatttggacggacgttgacggggtctatacctgcgacccgcgtcaggtgcccgatgcgaggttgttgaagtcgatgtcctaccaggaagcgatggagctttcctacttcggcgctaaagttcttcacccccgcaccattacccccatcgcccagttccagatcccttgcctgattaaaaataccggaaatcctcaagcaccaggtacgctcattggtgccagccgtgatgaagacgaattaccggtcaagggcatttccaatctgaataacatggcaatgttcagcgtttctggtccggggatgaaagggatggtcggcatggcggcgcgcgtctttgcagcgatgtcacgcgcccgtattttcgtggtgctgattacgcaatcatcttccgaatacagcatcagtttctgcgttccacaaagcgactgtgtgcgagctgaacgggcaatgcaggaagagttctacctggaactgaaagaaggcttactggagccgctggcagtgacggaacggctggccattatctcggtggtaggtgatggtatgcgcaccttgcgtgggatctcggcgaaattctttgccgcactggcccgcgccaatatcaacattgtcgccattgctcagggatcttctgaacgctcaatctctgtcgtggtaaataacgatgatgcgaccactggcgtgcgcgttactcatcagatgctgttcaataccgatcaggttatcgaagtgtttgtgattggcgtcggtggcgttggcggtgcgctgctggagcaactgaagcgtcagcaaagctggctgaagaataaacatatcgacttacgtgtctgcggtgttgccaactcgaaggctctgctcaccaatgtacatggccttaatctggaaaactggcaggaagaactggcgcaagccaaagagccgtttaatctcgggcgcttaattcgcctcgtgaaagaatatcatctgctgaacccggtcattgttgactgcacttccagccaggcagtggcggatcaatatgccgacttcctgcgcgaaggtttccacgttgtcacgccgaacaaaaaggccaacacctcgtcgatggattactaccatcagttgcgttatgcggcggaaaaatcgcggcgtaaattcctctatgacaccaacgttggggctggattaccggttattgagaacctgcaaaatctgctcaatgcaggtgatgaattgatgaagttctccggcattctttctggttcgctttcttatatcttcggcaagttagacgaaggcatgagtttctccgaggcgaccacgctggcgcgggaaatgggttataccgaaccggacccgcgagatgatctttctggtatggatgtggcgcgtaaactattgattctcgctcgtgaaacgggacgtgaactggagctggcggatattgaaattgaacctgtgctgcccgcagagtttaacgccgagggtgatgttgccgcttttatggcgaatctgtcacaactcgacgatctctttgccgcgcgcgtggcgaaggcccgtgatgaaggaaaagttttgcgctatgttggcaatattgatgaagatggcgtctgccgcgtgaagattgccgaagtggatggtaatgatccgctgttcaaagtgaaaaatggcgaaaacgccctggccttctatagccactattatcagccgctgccgttggtactgcgcggatatggtgcgggcaatgacgttacagctgccggtgtctttgctgatctgctacgtaccctctcatggaagttaggagtctgataa

SEQ ID NO: 35 - DNA, 7849 bp, Thauera butanivorans
atgtcagcaaatatggcagtcaaacaggccttgaaggccaatccggtcccgagttccgtggatcctcaggaagtccacaaatggcttcaggatttcacttgggatttcaagggcaagaccgcgaagtatccgaccaagtatgagatggacgtcaatacgcgcgagcagttcaagctgactgccaaggagtacgcgcgcatggagtcgatcaaggaagagcgccagtacggcaccctgctcgatggtctcgaccgtctggatgcgggcaacaaggtgcatccgaaatggggcgaggtgatgaagctggtctccaacttcctcgagaccggcgaatacggcgcaatcgccggttctgctctgctgtgggacacggcccagtcaccggagcagcgcaacggttacctcgctcaggtgatcgacgaaatccgccatgtgaaccagaccgcgtacgtgaattactactacggcaagcactactacgaccccgccggccacaccaacatgcgccagcttcgggcgatcaaccccctgtaccccggtgtcaagcgcgccttcggggagggctttctcgcgggcgatgccgtcgagtcctccatcaacctccaattggtcggcgaagcctgcttcaccaatccgctcatcgtttcgcttaccgaatgggcggccgcgaacggcgatgaaatcacgccgaccgtgttcctgtcgatcgaaaccgatgaactgcgccatatggccaacggctatcagaccatcgtgtcgatcatgaacaatccggagacgatgaagtacctgcaaacggacctcgataacgcattctggacgcagcacaagttcctgacccccttcgtcggggtggcgctcgaatatggttccaagtacaaggtcgagccgtgggccaagtcctggaaccggtgggtgtacgaagactgggctggcatctggctgggccgactgcagcagttcggcgtcaaaacgccaaagtgcctgcccgacgccaagaaagacgccgtttgggcacaccacgatctcgcgctgctggcccttgccctgtggccgctgaccggcatccgcatggaactcccggatagcctggcgatggagtggttcgaggctaattaccccggttggtacaaccattacggcaagatctacgaggagtggcgcgctgccggcttcgaggatccgaagagcggcttctgtggtgcgctctggctgatggagcgtggtcacggcattttcgtcgaccatgcctcgggtttgccgttctgccccagcctcgccaaaagctctatcaagccgcggttcactgaatacaacggcaagcgctacgccttcgccgagccgtatggcgagcgccagtggctgctcgagcccgagcgctacgagttccagaacttcttcgagcagttcgaaggctgggaactctccgatctcgtcaaggcggcaggcggcgtgcgcagcgatggcaaaacgctgatcgcgcagccgcatcttcgcgacaccgacatgtggacgcttgacgatctgaagcggatcaacctgacgatccccgacccgatgaagatcctcaactggcaacccgtcgcccagtgagggttgggccggcgcacgccgccaggcgagagatgcgccggcggggaagacttgccgcccgcctacgagcgggcggacgtgccaacgaacgaacagcactatatggagtagataaatatgtcaacaaacatctttacgcgcgggatggtagacccggagcgccaagcctgcattcaggaggtcgtacccaaggcgccgctggaaaccaagcgtgaccatattcctttcgccaaacgcggctggcgccgactcaccgagtatgaagcggtgatgttgcatgcgcagaattcactggacgccgtcccgggcagccaggaggtgggtgaagtcgtacagaagtggccgggtggcaggccgaactacggcgtcgagtcgaccgcggccctctccagcaactggttccatttccgcgacccgtcgaagcgctggttcatgccctacgtgaagcagaagaacgaggaagggcagacagccgaacgcgcgatgaagagttgggccgagggcggcgacgcggaaatgatgaacgccgcgtggcgggagcacatcctggcccggcactacggcgcgttcgtctataacgagtacggtctgttcagcgcccattcaacaacagtttacggtggcctttctgatctgatcaaaacctggattgccgaggccgcgttcgacaagaacgacgccggtcagatgatccagatgcagcgcgtgctgttgagcaaggtgttccctggcttcgacgccgacctggccgaagccaagcaggcatggactgaggacaagtcatggaaacccgcccgcgagttcgtcgagcacatctgggccgagacctacgactgggtcgagcagctttgggcgatccacgccgtctacgaccatattttcgggcaatttgtccggcgcgaattcttccagcggctcggcggcatccatggagacacgctgacgcccttcattcagaaccaggcgctgacgtatcacctgcaagcccgcgacggcgtaacggcactgtgcttcaaatttctgatcgaagacgagccggtatacgcccaacacaacaggcgctacctgcgggcatggacagggcgctatctcccgcaagtgggccgcgcattgaaggccttcctggcaatctacaaggaggttccggtaaagatcgatggggtgacttgccgggagggcgtgcgcgccagtgtcgaacgcgtggtggacgactgggcggcgcggttcgctgaaccgatcaacttcaagttcaaccgcgcggcgttcatcgacgatgtgctgagtggctactaattcaggggcagaccgaatgtcaaacgtaaatgcataccacgctggcaccaatggtaaagaaggccaggacttcatcgatgactttttgagcgaagagaacagtgctctacccacgagcgaagccgtggttctggccctgatgaagaccgaagagatcgacgcggtcgtcgacgagatgatcaagccgcagatggaggacaacccgaccatcgccgtcgaggaccgcggtggatactggtggatcaaggccaacggaaagatcgtcatcgattgtgacgaagccaccgaactgctgggcaagaagtatacggtgtacgatttgctggtcaacgtcagcaccaccgttgggcgggcgatgaccctcggcaatcagttcatcatcaccaacgagctccttggtctcgaaaccaaggtcgaaagcgtttactgaggaggatagacgacatgtcgaaacaggtttggtacaacacaccggtgcgcgacgagtggatcgagaagatcactgcgatcaggacagcgcgcgaaggcaccgacatgctcgcccgcttccgcgcggagcatacggggccggatcgcacgacgtacgatctcaagaaggaatacaattggatcgagtcgcgcatcgagatgcgggtcagccagcttcatgccgaggccacggcctcggacgaagacctcctcaccaagacgatcgacggccgctgcgcaaaggaagtcgcagcggagtggctgaaaaaggctgcggacatcgactgtcactacgagatggagcggctttgtgtcgccttccgcaaggcgtgcaaaccgccgatgatgcccatcaacttcttcgcgccggcagagaaggagttggtggcgaagctcatgaagctgagggcgcccacgtatctgactacctccctcgacgagttgcgcgaggcacggggtgtaacgatgatttccgtgcagtaagcggccggacgttctgagccggatcctccccgacaacggggaggactggtcaaaatagccatcaagactgaaaggagcagggaatgccgaaatacatcatcgaacgttcgattccgggggcagggaagctgacggagcgggacctcgcgtcggtgtcgcagaaatcctgtggaatcctccgctccatggggccacaaatccaatgggtcaaaagctacgtcaccgacgacaagctctactgcatctatatcgctcccgatgaggcctccatccgcgagcatgcaagatcgggagactttcccgccgacaaggtgtcccgcatacatcggatgatcgacccgacttgcgcggaatcggcataatcgagccgtcctcggaggaccacgacttcccgagggcctccggacgatggatcgcggcggacggaaacctgcgtggccgcggacgcggggtggccacaagatcgtaacgtaaagtactctaatccgagagcgcggtcgctttcacccgatcgaggtggaagtgactgggcgagggggagatatgaaagaagcgccggcaatacccgatctgcccggtttgcccgagactgtcggcgaaccgacgctggtcctggaagaagatggcttccgggttttcgccacagagctgacgatcatgtggcgatgggacatctacaacggcgatgcgcacgttcataccggatgcgcccagcacccggagagctgcgtcgtcgcggcgagatccaaaattcgattcttacggcgccccactgtggccatgctactgggcggtgaaggtcaatgagagggggttcgagacgccaactgatggacgcgacaccctagtaggagactgaaggtatatgctgatgcaacaatataaaatagtcgcccgcttcgaagacggcgtgacatacgagtacgattgcggcgaggacgaaaacctgctcgccgcggcgttgcgccagaacgtccgtttgctgtgtcaatgcagaaaggcgttttgcggcagttgcaaggccttgtgcagcgagggcgactatgaactgggcgatcatatcaacgttcaggtcttgccgcctgacgaagaagaggacggggtggtggtcacctgcgataccttcccacgcagcgatctggtccttgagtttccgtataccagcgaccgtctcggtaccgttacggctaccgaggcgaagacgagcgtcgtcagcgtggagagactctctagcaccgtttaccgtctggtgctgcaggcactcgacgccgaaggaatgcctgcacggttcgacttcgttcccgggcagtatgtcgaaatcagtacagccgattcgctcgagaccagggcgttttcgctggcgaaccttccaaacgacgccggattgctcgagttcttgatccgactcgtccccggcggatactatgcggcctatctggagcaacgcgcagcggcagggcagacgatcaacgtgaagggaccgttcggcgagttcgtgctgcgtgaacacgagttggtggaggacttcacgctgccagcggacagcccagcgagaggtgggacgattgcgttcctggcgggtagcacggggctggcgcctctcgcgagcatgctgcgcgagctcgggcggcgaggattcaacggcgagtgtcacctcttcttcggcatgcaggacaccgcaacgatgttctacgagaaggaactccgggacatcaagcgaacgcttccggggctcacgctgcatctcgccctgatggtcccttctgccgaatgggaaggctaccgtggtaacgccgtagctgcgttcaaagaacatttcgccgccagtagccagatacccgagaacgtttacctgtgcgggcccggaccgatgatcgcggccgcgctcggcgcttgtcgcgagcttggaattcccgacaatagagttcacagggaagaattcgtagcgagcggcggttgaacggcgcttgaagaaaccggggcgcgacgggcagcgtttacgagaccatgagtcaagaagaaagagattttctggccggtgcagcggcaacactcggcgagagcgtcggacaactggagcgccttgcccaagaattaagtgaaaagctgggcgatgagcgtgtcaacattgttcggggtttgctcgaacaggtttatgaccgcgacgacgaagcggaagtgcgtgcatccctctgctatacggagagaaagctgttgtgggcgtgggtccgtttgaagagactgaagggactcaggctcagtattggtcgtggttcgatgagaaacattatatgagaagcagaacgtgatcagtttaaattgcaagaagacaaccacagggttaactgcgcacttggccctggtccgcggaatgaaggcgttggcggaattggttggaacaacgctggggccccagggccgccacgtcatgcttgcgcatcgggccggattggctccgcacgtcagcaaggacggcgtcgaggtcgcacgtcacctgtcgttgccggacagcgaggaagaacttggtgtccggttgctgcgcaacgcagccgtcgcagtgtccgagtcctttggcgacggcacctccactgcaaccgtttttacggcggatctggccgtgcgggcgctcaaactgatcggtgccggggcggataccctggaggtccgtcgaggtctgggtttggctgcctatgccgcgttggtcgccctgaacgacatggcccggcgcgccgaccggggaatgctcaccgccgttgcccaaaccgctgccaacggcgaccgtcgcgtggcggatctcctcgtggaggccttcgaaagggtcggggctgaaggcacgatcgaagtagaaatgggtaattcggtcgaggacgtccttgaagtcgcgcagggcagttatttcgacacggtgccgctcgtcacggccttgttgccgccaacagggcaggtcgagttcgcccggccacttatccttttccattgcgacgcgatcgagacggcggacgagattcttccggcgctggagctagctcgcagttcacggagacccttgctgattctggccgactcggtgggtatcgacgtcgagacgctgctcgtccggaatcaaaatgaaggcaccttggctgttgcggtcgtcagggcgccaatgtatggcgatacgcgtcgcgaggccctgcttgacctgacgagcaaattcggggggacggcgtttggaagggagggattcgtcgaattcgcactcaggtctttgggatcactgtcggaaggtgacttggggcaggcggacgaagcaatcctagaggcggatggtgtgactctgagaggcgccggaaacaatccgtctgcattggaagaccgaatcgcgctggtgcgtgctgaactcgatcgcggcgacgtttccgtgggcgactccccatcggcaaagctcgactacatcgagaagcgaaaggagcggctgaagttgctggctgcaggtagcgccaagctgcatatcggggggccgacggacgttgagatcaagacgcgtttgccgctcgccgagaacgctcatcgggcgctcttggcggccgcgaagtcgggagtgctgcccgggggcggcgtcgcaatgattcgtgcggctgaaaaggtgcagcaagagatggggcgacttgaaggcgacgttgcttctggggcatccattttcctgcagtcgcttgacacacccatccggtggattgcacgaaatgcggggctgcgcccggatgaagtgctcgctcgaaccctggcgaacgagtcggacttttacggactcaacgccatgactggccgatatggcgacttggctgaagacggggttctcgacgccctcgatatggtcaccgacgtgatacgcgtcgcagtcagcgtcgtcgggtcgatgttgggcgtcggcgccctggtgactcgcgcatcgcccaagcccgcgccagagcgcttcaagggcacggagcgggtacacgacaagttgatgcgcgagggcgggttcgatgaatga

SEQ ID NO: 36 - DNA, 1038 bp, Corynebacterium glutamicum
atgaccactgctgcaccccaagaatttaccgctgctgttgttgaaaaattcggtcatgacgtgaccgtgaaggatattgaccttccaaagccagggccacaccaggcattggtgaaggtactcacctccggcatctgccacaccgacctccacgccttggagggcgattggccagtaaagccggaaccaccattcgtaccaggacacgaaggtgtaggtgaagttgttgagctcggaccaggtgaacacgatgtgaaggtcggcgatattgtcggcaatgcgtggctctggtcagcgtgtggcacctgcgaatactgcatcaccggcagggaaactcagtgcaacgaagctgagtatggtggctacacccaaaatggatccttcggccagtacatgctggtggatacccgttacgccgctcgcatcccagacggcgtggactacctcgaagcagcaccaattctgtgtgcaggcgtgactgtctacaaggcactcaaagtctctgaaacccgcccgggccaattcatggtgatctccggtgtcggcggacttggccacatcgcagtccaatacgcagcggcgatgggcatgcgtgtcattgcggtagatattgccgatgacaagctggaacttgcccgtaagcacggtgcggaatttaccgtgaatgcgcgtaatgaagattcaggcgaagctgtacagaagtacaccaacggtggcgcacacggcgtgcttgtgactgcagttcacgaggcagcattcggccaggcactggatatggctcgacgtgcaggaacaattgtgttcaacggtctgccaccgggagagttcccagcatccgtgttcaacatcgtattcaagggcctgaccatccgtggatccctcgtgggaacccgccaagacttggccgaagcgctcgatttctttgcacgcggactaatcaagccaaccgtgagtgagtgctccctcgatgaggtcaatggtgtgcttgaccgcatgcgaaacggcaagatcgatggtcgtgtggcgattcgtttctaa

SEQ ID NO: 37 - DNA, 1521 bp, Corynebacterium glutamicum
atgactgtctacgcaaatccaggaaccgaaggctcgatcgttaactatgaaaagcgctacgagaactacattggtggcaagtgggttccaccggtagagggccagtaccttgagaacatttcacctgtcactggtgaagttttctgtgaggtcgcacgtggcaccgcagcggacgtggagcttgcactggatgctgcacatgcagccgctgatgcgtggggcaagacttctgtcgctgaacgtgctctgatcctgcaccgcattgcggaccgcatggaagagcacctggaagaaatcgcagttgcagaaacctgggagaacggcaaggcagtccgtgagactcttgctgcagatatcccactggcaatcgaccacttccgctactttgctggcgcgatccgtgctcaggaagatcgttcctcacagatcgaccacaacactgttgcttaccacttcaacgagccaatcggtgttgttggtcagatcattccttggaacttcccaatcctcatggctacctggaagctcgcaccggcacttgctgcaggtaacgcgatcgtcatgaagccagctgagcagaccccagcatccattttgtatctgattaacatcatcggcgatctcatcccagagggcgtcctcaacatcgtcaacggactcggcggtgaagcaggcgctgcactgtccggctctaatcggattggcaagattgctttcaccggttccaccgaggtcggcaagctgatcaaccgcgctgcatccgacaagatcattcctgtcaccctggagctcggcggtaagtccccatccatcttcttctccgatgttctgtcacaggatgacgccttcgcagagaaggcagttgaaggcttcgcgatgttcgccctcaatcagggtgaagtttgtacctgtccttcccgtgcacttgttcatgagtccatcgctgatgaattcctcgagcttggcgtgaagcgagttcagaacatcaagctgggtaacccacttgatactgaaaccatgatgggtgctcaggcgtcccaggagcagatggacaagatctcctcctacctgaagatcggcccagaagaaggcgctcaaaccctcactggtggcaaggtcaacaaggttgatggcatggagaacggttactacattgagccaaccgttttccgcggcaccaacgacatgaggatcttccgcgaggaaatcttcggaccagtcctttctgttgctaccttcagcgacttcgatgaggccatccgtattgcaaacgacaccaactacggcctcggcgctggtgtctggagccgtgaccaaaacaccatttatcgtgcaggtcgcgcaatccaggctggtcgagtttgggtcaaccagtaccacaactacccagcgcactccgctttcggtggatacaaggagtccggcatcggccgtgagaaccacctcatgatgctgaaccactaccagcagaccaagaacctgttggtctcctacgatccaaacccaaccggactgttctga

SEQ ID NO: 38 - DNA, 1134 bp, Corynebacterium glutamicum
atgcccaccctcgcgccttcaggtcaacttgaaatccaagcgatcggtgatgtctccaccgaagccggagcaatcattacaaacgctgaaatcgcctatcaccgctggggtgaataccgcgtagataaagaaggacgcagcaatgtcgttctcatcgaacacgccctcactggagattccaacgcagccgattggtgggctgacttgctcggtcccggcaaagccatcaacactgatatttactgcgtgatctgtaccaacgtcatcggtggttgcaacggttccaccggacctggctccatgcatccagatggaaatttctggggtaatcgcttccccgccacgtccattcgtgatcaggtaaacgccgaaaaacaattcctcgacgcactcggcatcaccacggtcgccgcagtacttggtggttccatgggtggtgcccgcaccctagagtgggccgcaatgtacccagaaactgttggcgcagctgctgttcttgcagtttctgcacgcgccagcgcctggcaaatcggcattcaatccgcccaaattaaggcgattgaaaacgaccaccactggcacgaaggcaactactacgaatccggctgcaacccagccaccggactcggcgccgcccgacgcatcgcccacctcacctaccgtggcgaactagaaatcgacgaacgcttcggcaccaaagcccaaaagaacgaaaacccactcggtccctaccgcaagcccgaccagcgcttcgccgtggaatcctacttggactaccaagcagacaagctagtacagcgtttcgacgccggctcctacgtcttgctcaccgacgccctcaaccgccacgacattggtcgcgaccgcggaggcctcaacaaggcactcgaatccatcaaagttccagtccttgtcgcaggcgtagataccgatattttgtacccctaccaccagcaagaacacctctccagaaacctgggaaatctactggcaatggcaaaaatcgtatcccctgtcggccacgatgctttcctcaccgaaagccgccaaatggatcgcatcgtgaggaacttcttcagcctcatctccccagacgaagacaacccttcgacctacatcgagttctacatctaa

SEQ ID NO: 39 - DNA, 6686 bp, Artificial Sequence
  vector
cctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtctcggtacccggggatcgccattccggctgatcacatggtgctgatggcaggtttcaccgccggtaatgaaaaaggcgaactggtggtgcttggacgcaacggttccgactactctgctgcggtgctggctgcctgtttacgcgccgattgttgcgagatttggacggacgttgacggggtctatacctgcgacccgcgtcaggtgcccgatgcgaggttgttgaagtcgatgtcctaccaggaagcgatggagctttcctacttcggcgctaaagttcttcacccccgcaccattacccccatcgcccagttccagatcccttgcctgattaaaaataccggaaatcctcaagcaccaggtacgctcattggtgccagccgtgatgaagacgaattaccggtcaagggcatttccaatctgaataacatggcaatgttcagcgtttctggtccggggatgaaagggatggtcggcatggcggcgcgcgtctttgcagcgatgtcacgcgcccgtattttcgtggtgctgattacgcaatcatcttccgaatacagcatcagtttctgcgttccacaaagcgactgtgtgcgagctgaacgggcaatgcaggaagagttctacctggaactgaaagaaggcttactggagccgctggcagtgacggaacggctggccattatctcggtggtaggtgatggtatgcgcaccttgcgtgggatctcggcgaaattctttgccgcactggcccgcgccaatatcaacattgtcgccattgctcagggatcttctgaacgctcaatctctgtcgtggtaaataacgatgatgcgaccactggcgtgcgcgttactcatcagatgctgttcaataccgatcaggttatcgaagtgtttgtgattggcgtcggtggcgttggcggtgcgctgctggagcaactgaagcgtcagcaaagctggctgaagaataaacatatcgacttacgtgtctgcggtgttgccaactcgaaggctctgctcaccaatgtacatggccttaatctggaaaactggcaggaagaactggcgcaagcgatcctctagagtcgaccggtggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcaccgcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgagttcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatattgactaccggaagcagtgtgaccgtgtgcttctcaaatgcctgattcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcggggatcgacgctctcccttatgcgactcctgca

SEQ ID NO: 40 - DNA, 6665 bp, Artificial Sequence
  vector
cctttcgtcttcgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtctcggtacccggggatcgctcacccagggatttatcggtagcgaaaataaaggtcgtacaacgacgcttggccgtggaggcagcgattatacggcagccttgctggcggaggctttacacgcatctcgtgttgatatctggaccgacgtcccgggcatctacaccaccgatccacgcgtagtttccgcagcaaaacgcattgatgaaatcgcgtttgccgaagcggcagagatggcaacttttggtgcaaaagtactgcatccggcaacgttgctacccgcagtacgcagcgatatcccggtctttgtcggctccagcaaagacccacgcgcaggtggtacgctggtgtgcaataaaactgaaaatccgccgctgttccgcgctctggcgcttcgtcgcaatcagactctgctcactttgcacagcctgaatatgctgcattctcgcggtttcctcgcggaagttttcggcatcctcgcgcggcataatatttcggtagacttaatcaccacgtcagaagtgagcgtggcattaatccttgataccaccggttcaacctccactggcgatacgttgctgacgcaatctctgctgatggagctttccgcactgtgtcgggtggaggtggaagaaggtctggcgctggtcgcgttgattggcaatgacctgtcaaaagcctgcggcgttggcaaagaggtattcggcgtactggaaccgttcaacattcgcatgatttgttatggcgcatccagccataacctgtgcttcctggtgcccggcgaagatgccgagcaggtggtgcaaaaactgcatagtaatttgtttgagtaaatactgtatggcctggaagctatatttcgggccgtattgattttcttgtcactatgctcatcaataaacgagcctgtactctgttaaccagcgtctttatcggagaataattgcctttaatttttttatctgcatctctaattaattatcgaaagagataaatagttaagagaaggcaaaatgaatattatcagttctgctcgcaaacgaattccgcgatcctctagagtcgaccggtggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcaccgcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgagttcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccaagcttcctgctgaacatcaaaggcaagaaaacatctgttgtcaaagacagcatccttgaacaaggacaattaacagttaacaaataaaaacgcaaaagaaaatgccgatattgactaccggaagcagtgtgaccgtgtgcttctcaaatgcctgattcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgaacgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttacccgtcttactgtcggggatcgacgctctcccttatgcgactcctgca

SEQ ID NO: 41 - DNA, 15214 bp, Artificial Sequence
  Plasmid HM-p-25
aaataattttgtttaactttaagaaggagatatacgatgtcagcaaatatggcagtcaaacaggccttgaaggccaatccggtcccgagttccgtggatcctcaggaagtccacaaatggcttcaggatttcacttgggatttcaagggcaagaccgcgaagtatccgaccaagtatgagatggacgtcaatacgcgcgagcagttcaagctgactgccaaggagtacgcgcgcatggagtcgatcaaggaagagcgccagtacggcaccctgctcgatggtctcgaccgtctggatgcgggcaacaaggtgcatccgaaatggggcgaggtgatgaagctggtctccaacttcctcgagaccggcgaatacggcgcaatcgccggttctgctctgctgtgggacacggcccagtcaccggagcagcgcaacggttacctcgctcaggtgatcgacgaaatccgccatgtgaaccagaccgcgtacgtgaattactactacggcaagcactactacgaccccgccggccacaccaacatgcgccagcttcgggcgatcaaccccctgtaccccggtgtcaagcgcgccttcggggagggctttctcgcgggcgatgccgtcgagtcctccatcaacctccaattggtcggcgaagcctgcttcaccaatccgctcatcgtttcgcttaccgaatgggcggccgcgaacggcgatgaaatcacgccgaccgtgttcctgtcgatcgaaaccgatgaactgcgccatatggccaacggctatcagaccatcgtgtcgatcatgaacaatccggagacgatgaagtacctgcaaacggacctcgataacgcattctggacgcagcacaagttcctgacccccttcgtcggggtggcgctcgaatatggttccaagtacaaggtcgagccgtgggccaagtcctggaaccggtgggtgtacgaagactgggctggcatctggctgggccgactgcagcagttcggcgtcaaaacgccaaagtgcctgcccgacgccaagaaagacgccgtttgggcacaccacgatctcgcgctgctggcccttgccctgtggccgctgaccggcatccgcatggaactcccggatagcctggcgatggagtggttcgaggctaattaccccggttggtacaaccattacggcaagatctacgaggagtggcgcgctgccggcttcgaggatccgaagagcggcttctgtggtgcgctctggctgatggagcgtggtcacggcattttcgtcgaccatgcctcgggtttgccgttctgccccagcctcgccaaaagctctatcaagccgcggttcactgaatacaacggcaagcgctacgccttcgccgagccgtatggcgagcgccagtggctgctcgagcccgagcgctacgagttccagaacttcttcgagcagttcgaaggctgggaactctccgatctcgtcaaggcggcaggcggcgtgcgcagcgatggcaaaacgctgatcgcgcagccgcatcttcgcgacaccgacatgtggacgcttgacgatctgaagcggatcaacctgacgatccccgacccgatgaagatcctcaactggcaacccgtcgcccagtgagggttgggccggcgcacgccgccaggcgagagatgcgccggcggggaagacttgccgcccgcctacgagcgggcggacgtgccaacgaacgaacagcactatatggagtagataaatatgtcaacaaacatctttacgcgcgggatggtagacccggagcgccaagcctgcattcaggaggtcgtacccaaggcgccgctggaaaccaagcgtgaccatattcctttcgccaaacgcggctggcgccgactcaccgagtatgaagcggtgatgttgcatgcgcagaattcactggacgccgtcccgggcagccaggaggtgggtgaagtcgtacagaagtggccgggtggcaggccgaactacggcgtcgagtcgaccgcggccctctccagcaactggttccatttccgcgacccgtcgaagcgctggttcatgccctacgtgaagcagaagaacgaggaagggcagacagccgaacgcgcgatgaagagttgggccgagggcggcgacgcggaaatgatgaacgccgcgtggcgggagcacatcctggcccggcactacggcgcgttcgtctataacgagtacggtctgttcagcgcccattcaacaacagtttacggtggcctttctgatctgatcaaaacctggattgccgaggccgcgttcgacaagaacgacgccggtcagatgatccagatgcagcgcgtgctgttgagcaaggtgttccctggcttcgacgccgacctggccgaagccaagcaggcatggactgaggacaagtcatggaaacccgcccgcgagttcgtcgagcacatctgggccgagacctacgactgggtcgagcagctttgggcgatccacgccgtctacgaccatattttcgggcaatttgtccggcgcgaattcttccagcggctcggcggcatccatggagacacgctgacgcccttcattcagaaccaggcgctgacgtatcacctgcaagcccgcgacggcgtaacggcactgtgcttcaaatttctgatcgaagacgagccggtatacgcccaacacaacaggcgctacctgcgggcatggacagggcgctatctcccgcaagtgggccgcgcattgaaggccttcctggcaatctacaaggaggttccggtaaagatcgatggggtgacttgccgggagggcgtgcgcgccagtgtcgaacgcgtggtggacgactgggcggcgcggttcgctgaaccgatcaacttcaagttcaaccgcgcggcgttcatcgacgatgtgctgagtggctactaattcaggggcagaccgaatgtcaaacgtaaatgcataccacgctggcaccaatggtaaagaaggccaggacttcatcgatgactttttgagcgaagagaacagtgctctacccacgagcgaagccgtggttctggccctgatgaagaccgaagagatcgacgcggtcgtcgacgagatgatcaagccgcagatggaggacaacccgaccatcgccgtcgaggaccgcggtggatactggtggatcaaggccaacggaaagatcgtcatcgattgtgacgaagccaccgaactgctgggcaagaagtatacggtgtacgatttgctggtcaacgtcagcaccaccgttgggcgggcgatgaccctcggcaatcagttcatcatcaccaacgagctccttggtctcgaaaccaaggtcgaaagcgtttactgaggaggatagacgacatgtcgaaacaggtttggtacaacacaccggtgcgcgacgagtggatcgagaagatcactgcgatcaggacagcgcgcgaaggcaccgacatgctcgcccgcttccgcgcggagcatacggggccggatcgcacgacgtacgatctcaagaaggaatacaattggatcgagtcgcgcatcgagatgcgggtcagccagcttcatgccgaggccacggcctcggacgaagacctcctcaccaagacgatcgacggccgctgcgcaaaggaagtcgcagcggagtggctgaaaaaggctgcggacatcgactgtcactacgagatggagcggctttgtgtcgccttccgcaaggcgtgcaaaccgccgatgatgcccatcaacttcttcgcgccggcagagaaggagttggtggcgaagctcatgaagctgagggcgcccacgtatctgactacctccctcgacgagttgcgcgaggcacggggtgtaacgatgatttccgtgcagtaagcggccggacgttctgagccggatcctccccgacaacggggaggactggtcaaaatagccatcaagactgaaaggagcagggaatgccgaaatacatcatcgaacgttcgattccgggggcagggaagctgacggagcgggacctcgcgtcggtgtcgcagaaatcctgtggaatcctccgctccatggggccacaaatccaatgggtcaaaagctacgtcaccgacgacaagctctactgcatctatatcgctcccgatgaggcctccatccgcgagcatgcaagatcgggagactttcccgccgacaaggtgtcccgcatacatcggatgatcgacccgacttgcgcggaatcggcataatcgagccgtcctcggaggaccacgacttcccgagggcctccggacgatggatcgcggcggacggaaacctgcgtggccgcggacgcggggtggccacaagatcgtaacgtaaagtactctaatccgagagcgcggtcgctttcacccgatcgaggtggaagtgactgggcgagggggagatatgaaagaagcgccggcaatacccgatctgcccggtttgcccgagactgtcggcgaaccgacgctggtcctggaagaagatggcttccgggttttcgccacagagctgacgatcatgtggcgatgggacatctacaacggcgatgcgcacgttcataccggatgcgcccagcacccggagagctgcgtcgtcgcggcgagatccaaaattcgattcttacggcgccccactgtggccatgctactgggcggtgaaggtcaatgagagggggttcgagacgccaactgatggacgcgacaccctagtaggagactgaaggtatatgctgatgcaacaatataaaatagtcgcccgcttcgaagacggcgtgacatacgagtacgattgcggcgaggacgaaaacctgctcgccgcggcgttgcgccagaacgtccgtttgctgtgtcaatgcagaaaggcgttttgcggcagttgcaaggccttgtgcagcgagggcgactatgaactgggcgatcatatcaacgttcaggtcttgccgcctgacgaagaagaggacggggtggtggtcacctgcgataccttcccacgcagcgatctggtccttgagtttccgtataccagcgaccgtctcggtaccgttacggctaccgaggcgaagacgagcgtcgtcagcgtggagagactctctagcaccgtttaccgtctggtgctgcaggcactcgacgccgaaggaatgcctgcacggttcgacttcgttcccgggcagtatgtcgaaatcagtacagccgattcgctcgagaccagggcgttttcgctggcgaaccttccaaacgacgccggattgctcgagttcttgatccgactcgtccccggcggatactatgcggcctatctggagcaacgcgcagcggcagggcagacgatcaacgtgaagggaccgttcggcgagttcgtgctgcgtgaacacgagttggtggaggacttcacgctgccagcggacagcccagcgagaggtgggacgattgcgttcctggcgggtagcacggggctggcgcctctcgcgagcatgctgcgcgagctcgggcggcgaggattcaacggcgagtgtcacctcttcttcggcatgcaggacaccgcaacgatgttctacgagaaggaactccgggacatcaagcgaacgcttccggggctcacgctgcatctcgccctgatggtcccttctgccgaatgggaaggctaccgtggtaacgccgtagctgcgttcaaagaacatttcgccgccagtagccagatacccgagaacgtttacctgtgcgggcccggaccgatgatcgcggccgcgctcggcgcttgtcgcgagcttggaattcccgacaatagagttcacagggaagaattcgtagcgagcggcggttgaacggcgcttgaagaaaccggggcgcgacgggcagcgtttacgagaccatgagtcaagaagaaagagattttctggccggtgcagcggcaacactcggcgagagcgtcggacaactggagcgccttgcccaagaattaagtgaaaagctgggcgatgagcgtgtcaacattgttcggggtttgctcgaacaggtttatgaccgcgacgacgaagcggaagtgcgtgcatccctctgctatacggagagaaagctgttgtgggcgtgggtccgtttgaagagactgaagggactcaggctcagtattggtcgtggttcgatgagaaacattatatgagaagcagaacgtgatcagtttaaattgcaagaagacaaccacagggttaactgcgcacttggccctggtccgcggaatgaaggcgttggcggaattggttggaacaacgctggggccccagggccgccacgtcatgcttgcgcatcgggccggattggctccgcacgtcagcaaggacggcgtcgaggtcgcacgtcacctgtcgttgccggacagcgaggaagaacttggtgtccggttgctgcgcaacgcagccgtcgcagtgtccgagtcctttggcgacggcacctccactgcaaccgtttttacggcggatctggccgtgcgggcgctcaaactgatcggtgccggggcggataccctggaggtccgtcgaggtctgggtttggctgcctatgccgcgttggtcgccctgaacgacatggcccggcgcgccgaccggggaatgctcaccgccgttgcccaaaccgctgccaacggcgaccgtcgcgtggcggatctcctcgtggaggccttcgaaagggtcggggctgaaggcacgatcgaagtagaaatgggtaattcggtcgaggacgtccttgaagtcgcgcagggcagttatttcgacacggtgccgctcgtcacggccttgttgccgccaacagggcaggtcgagttcgcccggccacttatccttttccattgcgacgcgatcgagacggcggacgagattcttccggcgctggagctagctcgcagttcacggagacccttgctgattctggccgactcggtgggtatcgacgtcgagacgctgctcgtccggaatcaaaatgaaggcaccttggctgttgcggtcgtcagggcgccaatgtatggcgatacgcgtcgcgaggccctgcttgacctgacgagcaaattcggggggacggcgtttggaagggagggattcgtcgaattcgcactcaggtctttgggatcactgtcggaaggtgacttggggcaggcggacgaagcaatcctagaggcggatggtgtgactctgagaggcgccggaaacaatccgtctgcattggaagaccgaatcgcgctggtgcgtgctgaactcgatcgcggcgacgtttccgtgggcgactccccatcggcaaagctcgactacatcgagaagcgaaaggagcggctgaagttgctggctgcaggtagcgccaagctgcatatcggggggccgacggacgttgagatcaagacgcgtttgccgctcgccgagaacgctcatcgggcgctcttggcggccgcgaagtcgggagtgctgcccgggggcggcgtcgcaatgattcgtgcggctgaaaaggtgcagcaagagatggggcgacttgaaggcgacgttgcttctggggcatccattttcctgcagtcgcttgacacacccatccggtggattgcacgaaatgcggggctgcgcccggatgaagtgctcgctcgaaccctggcgaacgagtcggacttttacggactcaacgccatgactggccgatatggcgacttggctgaagacggggttctcgacgccctcgatatggtcaccgacgtgatacgcgtcgcagtcagcgtcgtcgggtcgatgttgggcgtcggcgccctggtgactcgcgcatcgcccaagcccgcgccagagcgcttcaagggcacggagcgggtacacgacaagttgatgcgcgagggcgggttcgatgaatgaaatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttattagataattccttgacgctcagcttcaattgttgcttgcgtgcgattcactacattcaaggtggcaaatattttcctcatatgccactttatagcatcttcggtgacatgcatatttgttgctatttgtttgtttgagcacccctcttttacaagcctcaagacagcaatctgcttccgtgtcaataaagcgtcagctttattctctgcggactttccaatctcaactattcgcggaagactaaaagccccaatcgcttgatctaaattaactgctgtgaaggcttcacatgaagccggtattattcgctcaattaaacatacttcatcaagaactgtttgaaagcattgaagctgttttgctatctccactgcataaacaatgttaagctgagccttttttaaatcaccggcacctgcctgcgctccggccaaacacaataatccacggacttccagctggcccgcgttaattttacgggcttgctgaatagccaataacgctctgtgcgcggcactatgaaagttccgatctcgggaaagcactagtgattgaacaagcagcaggcgtgcttttaggggggctgagtgctgtccggagaaaatcttatgatcttcaagagtttttaaattatttatgcccgttatgccttgacagactaagcgctgatagatctcaatttggctcataacttccaatcttggtagatttttttcaaccgcatgcgccttcgcccactccaatatctcaatggagccatttaggtcactccttccaagccgccaagctgacacagcacggcatacggaaaaaaacacgtctgtcaccccgtgattggaaatgaactctaaaattttggagagcttttcttctgaggtgtccaagcagcgcaattcataatgtaactcaagctctagagcgtcaaacattttcgaagtaaactcggattccatcatctgcgcgcgactgtctgtgcgtgcttgagttataatctgcctcgcccagcccatttttccgcttgctagggcttgttgaaacctcgcgacatacagccaaccaaaagcaaaattttgttttgcaaatttattcacggcttgggcctgagccagcaccttctccaactctgcaaatctatactcactggcaaaaataaaagccaaacaggttagcgcggccccttttccaactgcgtttgaatccccaaataaactaatccacttattacagagctcctcactcgaaagcatttcatctttcgttgctttacctattgcaagcacaagctgcagccattccttttcttgccatttattttttttatcggattgtgaagataggtctttaattaacttctctgctcgcgcgccttgctgactgaaatacaatacccacgcgtaactaataagcactatgggttttttgtgccaggcctgcttcggcagctctaacagccactgtctcagcgcatctatttcgccctgacgaaatgacaaatctaaaattattctctcagacatgctgactgcccagcgacagtcattcgcccgtagggatattcgtattgcatactggtattcacctctacgccaatgccagaaagctgcacgcttaagcaggtaggatcttttagcaggattttcagtccaagtaatttctcgtagaaaattacgcagtactggatgcagtgtaaactgcgctggctcaccgctcacatggcgaagcaacatgtaattagtgcttaaatacttaatacatgagaccccattgacgcatttgaatacataattgtattgatcaggcgtcacgaaatcgagcaatgaagaatttgcaagaaaaacacgatagcgctcgggaatcgcctcaaatatttcatccctaaagtaattgtctacttcaactactgctgaaatatgcttggccggcaactcacgctttaacaaaaaaactacaagagcaggccacccctcaacttcttgcaccaaggtctctatctgttcttcaggaactccaagaacagactctgcctccgctaacgccaccgcctcttctgcgctaaaggccaagtctttctcggtgtactcccgcatagcgcctgcaagtttaagctgcgagaacccttttattgtattgcctgcaactgcaaacctgatattttttggtgtatttaacataaactccataagtgcgtgcaacaacggcaagtctaagtcatgattaatattatccaaacaaactagcgtttctatctcgttattcgaggtgctctgccaaagactagatgcaaggtctcgcaagagcgcaggcttgctcacaccctctctcacacggctgaattttaccatttcgaaagtttcaagctgctcaataatctctgcgcagatatcaaattcactgtaagaactggctcttaaagaaagccacactgcaggacgtccggctgttctgtggcgtagccactcgaacgcaagagcaacggttttcccatatccaggtggggctctgtaaaggcatactctgggagcggctccatccgcgatactcaatcttggccgatatatgcaactatgaactttggcacttactagagtcgtaatttgatccgctccgaccttagcgaccgggaaatcattatttattattattttcattatgctattctcgcgccagctgactggaaattttcaccataggttacggtgttaaatattaaaactacacttaagtgtagtcggcatgatcggtggtgcaaaatatttactagggaaggtctgaagtaggccgctatttctggccgacttcggccttcgccgattttgaagacgggcaccgggtcaaaatcgaccagatagctcgctcatttcggtgctttcagccgtcgcgagtagctcgcggtacctggcatgcttgcggccagctcgtgtttttccagcagacgacggagcaaaaactacccgtaggtgtagttggcgcaagcgtccgattagctcaggtttaagatgtcgagagtgagagtgggcggcttaactttctcagttaggcataaaattacgtcttaaatctcgtagcgactaatttaataaaaatt

SEQ ID NO: 42 - protein, 506 aa, Corynebacterium glutamicum
MTVYANPGTEGSIVNYEKRYENYIGGKWVPPVEGQYLENISPVTGEVFCEVARGTAADVELALDAAHAAADAWGKTSVAERALILHRIADRMEEHLEEIAVAETWENGKAVRETLAADIPLAIDHFRYFAGAIRAQEDRSSQIDHNTVAYHFNEPIGVVGQIIPWNFPILMATWKLAPALAAGNAIVMKPAEQTPASILYLINIIGDLIPEGVLNIVNGLGGEAGAALSGSNRIGKIAFTGSTEVGKLINRAASDKIIPVTLELGGKSPSIFFSDVLSQDDAFAEKAVEGFAMFALNQGEVCTCPSRALVHESIADEFLELGVKRVQNIKLGNPLDTETMMGAQASQEQMDKISSYLKIGPEEGAQTLTGGKVNKVDGMENGYYIEPTVFRGTNDMRIFREEIFGPVLSVATFSDFDEAIRIANDTNYGLGAGVWSRDQNTIYRAGRAIQAGRVWVNQYHNYPAHSAFGGYKESGIGRENHLMMLNHYQQTKNLLVSYDPNPTGLF

SEQ ID NO: 43 - DNA, 42 bp, Artificial Sequence
  primer_MW_15_66_f
tctcggtacccggggatcgctcacccagggatttatcggtag

SEQ ID NO: 44 - DNA, 22 bp, Artificial Sequence
  primer_MW_15_67_r
caaggattaatgccacgctcac

SEQ ID NO: 45 - DNA, 22 bp, Artificial Sequence
  primer_MW_15_68_f
gtgagcgtggcattaatccttg

SEQ ID NO: 46 - DNA, 42 bp, Artificial Sequence
  primer_MW_15_69_r
ggtcgactctagaggatcgcggaattcgtttgcgagcagaac

SEQ ID NO: 47 - DNA, 45 bp, Artificial Sequence
  primer_JC-15-009_fw
gctggtctcggtacccggggatcgccattccggctgatcacatgg

SEQ ID NO: 48 - DNA, 25 bp, Artificial Sequence
  primer_JC-15-006_rv
gtaatcagcaccacgaaaatacggg

SEQ ID NO: 49 - DNA, 25 bp, Artificial Sequence
  primer_JC-15-007_fw
cccgtattttcgtggtgctgattac

SEQ ID NO: 50 - DNA, 45 bp, Artificial Sequence
  primer_JC-15-010_rv
ccaccggtcgactctagaggatcgcttgcgccagttcttcctgcc

SEQ ID NO: 51 - protein, 820 aa, Escherichia coli
MRVLKFGGTSVANAERFLRVADILESNARQGQVATVLSAPAKITNHLVAMIEKTISGQDALPNISDAERIFAELLTGLAAAQPGFPLAQLKTFVDQEFAQIKHVLHGISLLGQCPDSINAALICRGEKMSIAIMAGVLEARGHNVTVIDPVEKLLAVGHYLESTVDIAESTRRIAASRIPADHMVLMAGFTAGNEKGELVVLGRNGSDYSAAVLAACLRADCCEIWTDVDGVYTCDPRQVPDARLLKSMSYQEAMELSYFGAKVLHPRTITPIAQFQIPCLIKNTGNPQAPGTLIGASRDEDELPVKGISNLNNMAMFSVSGPGMKGMVGMAARVFAAMSRARISVVLITQSSSEYSISFCVPQSDCVRAERAMQEEFYLELKEGLLEPLAVTERLAIISVVGDGMRTLRGISAKFFAALARANINIVAIAQGSSERSISVVVNNDDATTGVRVTHQMLFNTDQVIEVFVIGVGGVGGALLEQLKRQQSWLKNKHIDLRVCGVANSKALLTNVHGLNLENWQEELAQAKEPFNLGRLIRLVKEYHLLNPVIVDCTSSQAVADQYADFLREGFHVVTPNKKANTSSMDYYHQLRYAAEKSRRKFLYDTNVGAGLPVIENLQNLLNAGDELMKFSGILSGSLSYIFGKLDEGMSFSEATTLAREMGYTEPDPRDDLSGMDVARKLLILARETGRELELADIEIEPVLPAEFNAEGDVAAFMANLSQLDDLFAARVAKARDEGKVLRYVGNIDEDGVCRVKIAEVDGNDPLFKVKNGENALAFYSHYYQPLPLVLRGYGAGNDVTAAGVFADLLRTLSWKLGV

SEQ ID NO: 52 - DNA, 1134 bp, Corynebacterium glutamicum; identical to SEQ ID NO: 38

SEQ ID NO: 53 - DNA, 167 bp, Artificial Sequence
  tac promoter
atgttttacctcctgttaaacaaaattatttctagagggaaaccgttgtggaattgtgagcgctcacaattccacatccacacattatacgagccgatgattaattgtcaacagcgtcgatcactgtgcatgaagctcgtaattgttatccgctcacaattggatcc

SEQ ID NO: 54 - DNA, 43 bp, Artificial Sequence
  primer_metX_Cg
ggatctaggaaccaaggagagtggcatgcccaccctcgcgcct

SEQ ID NO: 55 - DNA, 34 bp, Artificial Sequence
  primer_metX_Cg
caattggatccgtttatccggagggttgcctgtg

SEQ ID NO: 56 - DNA, 38 bp, Artificial Sequence
  primer_tac promotor
accctccggataaacggatccaattgtgagcggataac

SEQ ID NO: 57 - DNA, 31 bp, Artificial Sequence
  primer_tac promotor
acactcgcatatgttttacctcctgttaaac

SEQ ID NO: 58 - DNA, 35 bp, Artificial Sequence
  primer_thrA part 1
caggaggtaaaacatatgcgagtgttgaagttcgg

SEQ ID NO: 59 - DNA, 25 bp, Artificial Sequence
  primer_thrA part 1
gtaatcagcaccacgaaaatacggg

SEQ ID NO: 60 - DNA, 25 bp, Artificial Sequence
  primer_thrA part 2
cccgtattttcgtggtgctgattac

SEQ ID NO: 61 - DNA, 50 bp, Artificial Sequence
  primer_thrA part 2
ggtgcgccaggagagttgttgatttatcagactcctaacttccatgagag

SEQ ID NO: 62 - DNA, 7052 bp, Artificial Sequence
  plasmid_pJ281_alaT_C.gl._TA_C.v.(Ct)
gtataggaacttctgaagtggggggatccggccggcccaaaaaggccgggaaatacccagcctcgctttgtaacggagtagagacgaaagtgattgcgcctacccggatattatcgtgaggatgcgtcatcgccattaattcactgatcagtgataagctgtcaaacatgagaattaattccggcgatccgtcgacttgcagcaattcccgaggctgtagccgacgatggtgcgccaggagagttgttgatctagattattaggcgaggccacgcgctttgagggtctgttcaaattcttcgaggcaacgttccgccacggcgagcatttcatccacttccgcacgggtcataacgagcggcggtgcagacacaatgtgatcgccgcacgcacgcataatgaggttgttgcgaaaaaaaatatcgcggcagagggtgccaatttcgccaaaatccggaaagagttcacgtttcgctttgtttttcacgagggtaaacgcctgcaccatgcccacgccacgcacatcatccacatgttcaaaacggctaaaggtttcacgccaacgtttctgcatatacgggccaatatcatctttcacacgctgcacaatgccttcatcacggagcgccgcaacattcgcatgcgccaccgccgcacacaccggatggccgctataggtaaagccatggttaaaatcaccgcccgcaatgagaccttccgcaacacgtttgcccacaaacaccgcgccaatcgggagatagccgctgctgaggcctttcgccgcggtaaagaggtccggctgaaagccaaaatgctgatggccaaaccattcgccggtacggccaaagccgcaaatcacttcatccgcaacgaggagcacatcatatttgcggcaaatacgttcaatttccggccaataggttgccggcggaacaatcacaccgcccgcaccctgaatcggttcgcccacaaacgccgccactttatccgcgccgatttcgagaattttttcttcgagccaacgcgccgcaaccacgccaaattcatccggggtcatatctttgccatgtttataccaccacggctgttcaatatgcgccatgcccggaatcgggagatcgccctgttcatgcatatatttcatgccgccgaggctcgcaccgccaatggtgctgccgtgatagccgttccaacggccaatgagggtttttttttccggtttgccctgcacatcccaataacgacgcaccatacgaatcatggtatccacgctttcgctgccgctgttggtataaaacacacgatcaaaacctgccggggtaacttcggcgaggaggctgctgagttccaccaccgccggatgggtggttttaaagaaggtgttataaaacgggagttcttccatctgacgacgcgccgcttccgcaaaatctttacggccatagcccacgttcacgcaccagaggcccgccatgccatcaataattttgttgccttcgctatcccagagatacacgccttcgccacgggtcatcacacgcgcacccgcctgattgaggctcgcggtatcggtaaacggatggagatgatgcgccgcatcgagttcacgccactgagaggtggtacgctgtttctgcatattatatctccttaaagcttttactactgcttgtaagtggacaggaagttacccaggcgctcaattgcgttttccaactgggatgcccatggcagggtgaccactcggaagtgatcgtgatgtggccagttgaagccagtgccctgaaccatgaggattttctcggcacggagaagatccagcatgagttgggtgtcgtcgtggatttcgtacacgttggggtcgagcttggggaacgcgtatagagctcccattggtttcacacagctgacacctgggatttcgttgagtttcgtccatgccatgttgcgctgttccaggagtcggccgtgttcgccagtgaggtcgtagatggactggcgtccaccgagagctacctgaatagcgtgctgagctgggacatttgggcagagtcgagtgcctgcgaggagttcgaggccctcaataaatccacgtgcgtattgctttggtccagtcaataccatccagccagctcggtatcctgcgacgcggtatgccttggatagaccgttgtatgtgatgcaaaggagatctggtgcaagggttgccaggctgatgtgctcggcatcatcgtagagaatgcggtcgtagatttcatcggccaaaatcagcaggtcatgctcgcgtgcaatctcgacgatttgttccaacacccggcgcgggtagacagctcccgtggggttgttggggttgatcaccacaatagctttggttttctctgagattttggacttgatgtcttcgatggatgggttccagtcatcttcctcatcacagaggtagtgcacaggcttaccaccagccagggaggttgcggcagtccacagtgggtagtccggtgcggggataagaacttcatcgccgtcgttgaggagtgcttgggtggtcatggtgattagttctgagacaccgttgcctaagaacacatcatcaacatcgaagtgggggaatccgggcacaacttcgtagcgggtgaccactgctcgccgggccggaataatgcctttggaggtggaatacccttgggaagttggaaggttggcgatcatgtcacgcataatcacgtcgggggcatcgaatccgaacacggctggatttcccgtgttgagctttaagatgttatgcccatcaagctccatgcgttccgcctccgcggccaccgggccacggatctcgtacagcacgtccttcatcttctccgactgatcgaagatgcggcgagttgttcgccgagtgggacgcgctgcctgatccgcgcccacagccttgttggcggtgtcggtggtcttagaggttttgcgcttgtctgtagtcatatgccactctccttggttcctagatcctgtgtgaaattgttattgttatccgctcacaattccacacattatacgagccggaagcataaagtgtcaagcctggggtgcctaatgagtgagctaacttacattaattgcgttgcgctcactgcccgcatatctatgagccgggctgaatgatcgaccgagacaggccctgcggggctgcaggccggccggatccaaaatgaagtgaagttcctatacttactagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagtcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactctcttccgggcgctatcatgccataccgcgaaaggttttgcgccattcgatggcgcgccgcttttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagt

SEQ ID NO: 63 - DNA, 8155 bp, Artificial Sequence
  plasmid_pJAG-4-48
actagagaataggaacttcactatagagtcgaataagggcgacaccccctaattagcccgggcgaaaggcccagtctttcgactgagcctttcgttttatttgatgcctggcagttccctactctcgcatggggagtccccacactaccatcggcgctacggcgtttcacttctgagttcggcatggggtcaggtgggaccaccgcgctactgccgccaggcaaacaaggggtgttatgagccatattcaggtataaatgggctcgcgataatgttcagaattggttaattggttgtaacactgacccctatttgtttatttttctaaatacattcaaatatgtatccgctcatgagacaataaccctgataaatgcttcaataatattgaaaaaggaagaatatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgccacttccgaccatcaagcattttatccgtactcctgatgatgcatggttactcaccactgcgatccccggaaaaacagcgttccaggtattagaagaatatcctgattcaggtgaaaatattgttgatgcgctggcagtgttcctgcgccggttgcactcgattcctgtttgtaattgtccttttaacagcgatcgcgtatttcgcctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaaaagcggcgcgccatcgaatggcgcaaaacctttcgcggtatggcatgatagcgcccggaagagagtcaattcagggtggtgaatatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaggacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagatagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaaaagaaaaaccaccctggcgcccaatacgcaaaccgcctctccccgcgcgttggccgattcattaatgcagctggcacgacaggtttcccgactggaaagcgggcagtgactcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaataaattttgtgtcgcccttattcgactcactatagaagttcctattctctagtaagtataggaacttcacttcattttggatccggccggcctgcagccccgcagggcctgtctcggtcgatcattcagcccggctcatagatatgcgggcagtgagcgcaacgcaattaatgtaagttagctcactcattaggcaccccaggcttgacactttatgcttccggctcgtataatgtgtggaattgtgagcggataacaataacaatttcacacaggatctaggaaccaaggagagtggcatgcccaccctcgcgccttcaggtcaacttgaaatccaagcgatcggtgatgtctccaccgaagccggagcaatcattacaaacgctgaaatcgcctatcaccgctggggtgaataccgcgtagataaagaaggacgcagcaatgtcgttctcatcgaacacgccctcactggagattccaacgcagccgattggtgggctgacttgctcggtcccggcaaagccatcaacactgatatttactgcgtgatctgtaccaacgtcatcggtggttgcaacggttccaccggacctggctccatgcatccagatggaaatttctggggtaatcgcttccccgccacgtccattcgtgatcaggtaaacgccgaaaaacaattcctcgacgcactcggcatcaccacggtcgccgcagtacttggtggttccatgggtggtgcccgcaccctagagtgggccgcaatgtacccagaaactgttggcgcagctgctgttcttgcagtttctgcacgcgccagcgcctggcaaatcggcattcaatccgcccaaattaaggcgattgaaaacgaccaccactggcacgaaggcaactactacgaatccggctgcaacccagccaccggactcggcgccgcccgacgcatcgcccacctcacctaccgtggcgaactagaaatcgacgaacgcttcggcaccaaagcccaaaagaacgaaaacccactcggtccctaccgcaagcccgaccagcgcttcgccgtggaatcctacttggactaccaagcagacaagctagtacagcgtttcgacgccggctcctacgtcttgctcaccgacgccctcaaccgccacgacattggtcgcgaccgcggaggcctcaacaaggcactcgaatccatcaaagttccagtccttgtcgcaggcgtagataccgatattttgtacccctaccaccagcaagaacacctctccagaaacctgggaaatctactggcaatggcaaaaatcgtatcccctgtcggccacgatgctttcctcaccgaaagccgccaaatggatcgcatcgtgaggaacttcttcagcctcatctccccagacgaagacaacccttcgacctacatcgagttctacatctaataggtatttacgacaaatagacagggatctctaaacaactcacaggcaaccctccggataaacggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggaggtaaaacatatgcgagtgttgaagttcggcggtacatcagtggcaaatgcagaacgttttctgcgtgttgccgatattctggaaagcaatgccaggcaggggcaggtggccaccgtcctctctgcccccgccaaaatcaccaaccacctggtggcgatgattgaaaaaaccattagcggccaggatgctttacccaatatcagcgatgccgaacgtatttttgccgaacttttgacgggactcgccgccgcccagccggggttcccgctggcgcaattgaaaactttcgtcgatcaggaatttgcccaaataaaacatgtcctgcatggcattagtttgttggggcagtgcccggatagcatcaacgctgcgctgatttgccgtggcgagaaaatgtcgatcgccattatggccggcgtattagaagcgcgcggtcacaacgttactgttatcgatccggtcgaaaaactgctggcagtggggcattacctcgaatctaccgtcgatattgctgagtccacccgccgtattgcggcaagccgcattccggctgatcacatggtgctgatggcaggtttcaccgccggtaatgaaaaaggcgaactggtggtgcttggacgcaacggttccgactactctgctgcggtgctggctgcctgtttacgcgccgattgttgcgagatttggacggacgttgacggggtctatacctgcgacccgcgtcaggtgcccgatgcgaggttgttgaagtcgatgtcctaccaggaagcgatggagctttcctacttcggcgctaaagttcttcacccccgcaccattacccccatcgcccagttccagatcccttgcctgattaaaaataccggaaatcctcaagcaccaggtacgctcattggtgccagccgtgatgaagacgaattaccggtcaagggcatttccaatctgaataacatggcaatgttcagcgtttctggtccggggatgaaagggatggtcggcatggcggcgcgcgtctttgcagcgatgtcacgcgcccgtattttcgtggtgctgattacgcaatcatcttccgaatacagcatcagtttctgcgttccacaaagcgactgtgtgcgagctgaacgggcaatgcaggaagagttctacctggaactgaaagaaggcttactggagccgctggcagtgacggaacggctggccattatctcggtggtaggtgatggtatgcgcaccttgcgtgggatctcggcgaaattctttgccgcactggcccgcgccaatatcaacattgtcgccattgctcagggatcttctgaacgctcaatctctgtcgtggtaaataacgatgatgcgaccactggcgtgcgcgttactcatcagatgctgttcaataccgatcaggttatcgaagtgtttgtgattggcgtcggtggcgttggcggtgcgctgctggagcaactgaagcgtcagcaaagctggctgaagaataaacatatcgacttacgtgtctgcggtgttgccaactcgaaggctctgctcaccaatgtacatggccttaatctggaaaactggcaggaagaactggcgcaagccaaagagccgtttaatctcgggcgcttaattcgcctcgtgaaagaatatcatctgctgaacccggtcattgttgactgcacttccagccaggcagtggcggatcaatatgccgacttcctgcgcgaaggtttccacgttgtcacgccgaacaaaaaggccaacacctcgtcgatggattactaccatcagttgcgttatgcggcggaaaaatcgcggcgtaaattcctctatgacaccaacgttggggctggattaccggttattgagaacctgcaaaatctgctcaatgcaggtgatgaattgatgaagttctccggcattctttctggttcgctttcttatatcttcggcaagttagacgaaggcatgagtttctccgaggcgaccacgctggcgcgggaaatgggttataccgaaccggacccgcgagatgatctttctggtatggatgtggcgcgtaaactattgattctcgctcgtgaaacgggacgtgaactggagctggcggatattgaaattgaacctgtgctgcccgcagagtttaacgccgagggtgatgttgccgcttttatggcgaatctgtcacaactcgacgatctctttgccgcgcgcgtggcgaaggcccgtgatgaaggaaaagttttgcgctatgttggcaatattgatgaagatggcgtctgccgcgtgaagattgccgaagtggatggtaatgatccgctgttcaaagtgaaaaatggcgaaaacgccctggccttctatagccactattatcagccgctgccgttggtactgcgcggatatggtgcgggcaatgacgttacagctgccggtgtctttgctgatctgctacgtaccctctcatggaagttaggagtctgataaatcaacaactctcctggcgcaccatcgtcggctacagcctcgggaattgctgcaagtcgacggatcgccggaattaattctcatgtttgacagcttatcactgatcagtgaattaatggcgatgacgcatcctcacgataatatccgggtaggcgcaatcactttcgtctctactccgttacaaagcgaggctgggtatttcccggcctttttgggccggccggatccccccacttcagaagttcctatac

SEQ ID NO: 64 - DNA, 3089 bp, Artificial Sequence
  DCPK induction system
tccaatttttattaaattagtcgctacgagatttaagacgtaattttatgcctaactgagaaagttaagccgcccactctcactctcgacatcttaaacctgagctaatcggacgcttgcgccaactacacctacgggtagtttttgctccgtcgtctgctggaaaaacacgagctggccgcaagcatgccaggtaccgcgagctactcgcgacggctgaaagcaccgaaatgagcgagctatctggtcgattttgacccggtgcccgtcttcaaaatcggcgaaggccgaagtcggccagaaatagcggcctacttcagaccttccctagtaaatattttgcaccaccgatcatgccgactacacttaagtgtagttttaatatttaacaccgtaacctatggtgaaaatttccagtcagctggcgcgagaatagcataatgaaaataataataaataatgatttcccggtcgctaaggtcggagcggatcaaattacgactctagtaagtgccaaagttcatagttgcatatatcggccaagattgagtatcgcggatggagccgctcccagagtatgcctttacagagccccacctggatatgggaaaaccgttgctcttgcgttcgagtggctacgccacagaacagccggacgtcctgcagtgtggctttctttaagagccagttcttacagtgaatttgatatctgcgcagagattattgagcagcttgaaactttcgaaatggtaaaattcagccgtgtgagagagggtgtgagcaagcctgcgctcttgcgagaccttgcatctagtctttggcagagcacctcgaataacgagatagaaacgctagtttgtttggataatattaatcatgacttagacttgccgttgttgcacgcacttatggagtttatgttaaatacaccaaaaaatatcaggtttgcagttgcaggcaatacaataaaagggttctcgcagcttaaacttgcaggcgctatgcgggagtacaccgagaaagacttggcctttagcgcagaagaggcggtggcgttagcggaggcagagtctgttcttggagttcctgaagaacagatagagaccttggtgcaagaagttgaggggtggcctgctcttgtagtttttttgttaaagcgtgagttgccggccaagcatatttcagcagtagttgaagtagacaattactttagggatgaaatatttgaggcgattcccgagcgctatcgtgtttttcttgcaaattcttcattgctcgatttcgtgacgcctgatcaatacaattatgtattcaaatgcgtcaatggggtctcatgtattaagtatttaagcactaattacatgttgcttcgccatgtgagcggtgagccagcgcagtttacactgcatccagtactgcgtaattttctacgagaaattacttggactgaaaatcctgctaaaagatcctacctgcttaagcgtgcagctttctggcattggcgtagaggtgaataccagtatgcaatacgaatatccctacgggcgaatgactgtcgctgggcagtcagcatgtctgagagaataattttagatttgtcatttcgtcagggcgaaatagatgcgctgagacagtggctgttagagctgccgaagcaggcctggcacaaaaaacccatagtgcttattagttacgcgtgggtattgtatttcagtcagcaaggcgcgcgagcagagaagttaattaaagacctatcttcacaatccgataaaaaaaataaatggcaagaaaaggaatggctgcagcttgtgcttgcaataggtaaagcaacgaaagatgaaatgctttcgagtgaggagctctgtaataagtggattagtttatttggggattcaaacgcagttggaaaaggggccgcgctaacctgtttggcttttatttttgccagtgagtatagatttgcagagttggagaaggtgctggctcaggcccaagccgtgaataaatttgcaaaacaaaattttgcttttggttggctgtatgtcgcgaggtttcaacaagccctagcaagcggaaaaatgggctgggcgaggcagattataactcaagcacgcacagacagtcgcgcgcagatgatggaatccgagtttacttcgaaaatgtttgacgctctagagcttgagttacattatgaattgcgctgcttggacacctcagaagaaaagctctccaaaattttagagttcatttccaatcacggggtgacagacgtgtttttttccgtatgccgtgctgtgtcagcttggcggcttggaaggagtgacctaaatggctccattgagatattggagtgggcgaaggcgcatgcggttgaaaaaaatctaccaagattggaagttatgagccaaattgagatctatcagcgcttagtctgtcaaggcataacgggcataaataatttaaaaactcttgaagatcataagattttctccggacagcactcagcccccctaaaagcacgcctgctgcttgttcaatcactagtgctttcccgagatcggaactttcatagtgccgcgcacagagcgttattggctattcagcaagcccgtaaaattaacgcgggccagctggaagtccgtggattattgtgtttggccggagcgcaggcaggtgccggtgatttaaaaaaggctcagcttaacattgtttatgcagtggagatagcaaaacagcttcaatgctttcaaacagttcttgatgaagtatgtttaattgagcgaataataccggcttcatgtgaagccttcacagcagttaatttagatcaagcgattggggcttttagtcttccgcgaatagttgagattggaaagtccgcagagaataaagctgacgctttattgacacggaagcagattgctgtcttgaggcttgtaaaagaggggtgctcaaacaaacaaatagcaacaaatatgcatgtcaccgaagatgctataaagtggcatatgaggaaaatatttgccaccttgaatgtagtgaatcgcacgcaagcaacaattgaagctgagcgtcaaggaattatctaa

SEQ ID NO: 65 - DNA, 48 bp, Artificial Sequence
  primer_BF_MD 27
cgtagcgactaatttaataaaaattggaagcaatttgccaagtaatcc

SEQ ID NO: 66 - DNA, 33 bp, Artificial Sequence
  primer_BF_MD 28
acctgagcactacttagaaacgaatcgccacac

SEQ ID NO: 67 - DNA, 40 bp, Artificial Sequence
  primer_BF_MD 29
attcgtttctaagtagtgctcaggtggaggtggcccaaag

SEQ ID NO: 68 - DNA, 47 bp, Artificial Sequence
  primer_BF_MD 30
gcagattgtactgagagtgcaccaaaaaagccgccgcacactggagg

SEQ ID NO: 69 - DNA, 9451 bp, Artificial Sequence
  plasmid_AH-p-125
gcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccaaaaaagccgccgcacactggaggatgtgtgaggcagcttttctaggacttaacgcttagatcagaacagtccggttgggtttggatcgtaggagaccaacaggttcttggtctgctggtagtggttcagcatcatgaggtggttctcacggccgatgccggactccttgtatccaccgaaagcggagtgcgctgggtagttgtggtactggttgacccaaactcgaccagcctggattgcgcgacctgcacgataaatggtgttttggtcacggctccagacaccagcgccgaggccgtagttggtgtcgtttgcaatacggatggcctcatcgaagtcgctgaaggtagcaacagaaaggactggtccgaagatttcctcgcggaagatcctcatgtcgttggtgccgcggaaaacggttggctcaatgtagtaaccgttctccatgccatcaaccttgttgaccttgccaccagtgagggtttgagcgccttcttctgggccgatcttcaggtaggaggagatcttgtccatctgctcctgggacgcctgagcacccatcatggtttcagtatcaagtgggttacccagcttgatgttctgaactcgcttcacgccaagctcgaggaattcatcagcgatggactcatgaacaagtgcacgggaaggacaggtacaaacttcaccctgattgagggcgaacatcgcgaagccttcaactgccttctctgcgaaggcgtcatcctgtgacagaacatcggagaagaagatggatggggacttaccgccgagctccagggtgacaggaatgatcttgtcggatgcagcgcggttgatcagcttgccgacctcggtggaaccggtgaaagcaatcttgccaatccgattagagccggacagtgcagcgcctgcttcaccgccgagtccgttgacgatgttgaggacgccctctgggatgagatcgccgatgatgttaatcagatacaaaatggatgctggggtctgctcagctggcttcatgacgatcgcgttacctgcagcaagtgccggtgcgagcttccaggtagccatgaggattgggaagttccaaggaatgatctgaccaacaacaccgattggctcgttgaagtggtaagcaacagtgttgtggtcgatctgtgaggaacgatcttcctgagcacggatcgcgccagcaaagtagcggaagtggtcgattgccagtgggatatctgcagcaagagtctcacggactgccttgccgttctcccaggtttctgcaactgcgatttcttccaggtgctcttccatgcggtccgcaatgcggtgcaggatcagagcacgttcagcgacagaagtcttgccccacgcatcagcggctgcatgtgcagcatccagtgcaagctccacgtccgctgcggtgccacgtgcgacctcacagaaaacttcaccagtgacaggtgaaatgttctcaaggtactggccctctaccggtggaacccacttgccaccaatgtagttctcgtagcgcttttcatagttaacgatcgagccttcggttcctggatttgcgtagacagtcattgggtctcctttgggccacctccacctgagcactacttagaaacgaatcgccacacgaccatcgatcttgccgtttcgcatgcggtcaagcacaccattgacctcatcgagggagcactcactcacggttggcttgattagtccgcgtgcaaagaaatcgagcgcttcggccaagtcttggcgggttcccacgagggatccacggatggtcaggcccttgaatacgatgttgaacacggatgctgggaactctcccggtggcagaccgttgaacacaattgttcctgcacgtcgagccatatccagtgcctggccgaatgctgcctcgtgaactgcagtcacaagcacgccgtgtgcgccaccgttggtgtacttctgtacagcttcgcctgaatcttcattacgcgcattcacggtaaattccgcaccgtgcttacgggcaagttccagcttgtcatcggcaatatctaccgcaatgacacgcatgcccatcgccgctgcgtattggactgcgatgtggccaagtccgccgacaccggagatcaccatgaattggcccgggcgggtttcagagactttgagtgccttgtagacagtcacgcctgcacacagaattggtgctgcttcgaggtagtccacgccgtctgggatgcgagcggcgtaacgggtatccaccagcatgtactggccgaaggatccattttgggtgtagccaccatactcagcttcgttgcactgagtttccctgccggtgatgcagtattcgcaggtgccacacgctgaccagagccacgcattgccgacaatatcgccgaccttcacatcgtgttcacctggtccgagctcaacaacttcacctacaccttcgtgtcctggtacgaatggtggttccggctttactggccaatcgccctccaaggcgtggaggtcggtgtggcagatgccggaggtgagtaccttcaccaatgcctggtgtggccctggctttggaaggtcaatatccttcacggtcacgtcatgaccgaatttttcaacaacagcagcggtaaattcttggggtgcagcagtggtcataaaactcactccttctcgcttggattacttggcaaattgcttccaatttttattaaattagtcgctacgagatttaagacgtaattttatgcctaactgagaaagttaagccgcccactctcactctcgacatcttaaacctgagctaatcggacgcttgcgccaactacacctacgggtagtttttgctccgtcgtctgctggaaaaacacgagctggccgcaagcatgccaggtaccgcgagctactcgcgacggctgaaagcaccgaaatgagcgagctatctggtcgattttgacccggtgcccgtcttcaaaatcggcgaaggccgaagtcggccagaaatagcggcctacttcagaccttccctagtaaatattttgcaccaccgatcatgccgactacacttaagtgtagttttaatatttaacaccgtaacctatggtgaaaatttccagtcagctggcgcgagaatagcataatgaaaataataataaataatgatttcccggtcgctaaggtcggagcggatcaaattacgactctagtaagtgccaaagttcatagttgcatatatcggccaagattgagtatcgcggatggagccgctcccagagtatgcctttacagagccccacctggatatgggaaaaccgttgctcttgcgttcgagtggctacgccacagaacagccggacgtcctgcagtgtggctttctttaagagccagttcttacagtgaatttgatatctgcgcagagattattgagcagcttgaaactttcgaaatggtaaaattcagccgtgtgagagagggtgtgagcaagcctgcgctcttgcgagaccttgcatctagtctttggcagagcacctcgaataacgagatagaaacgctagtttgtttggataatattaatcatgacttagacttgccgttgttgcacgcacttatggagtttatgttaaatacaccaaaaaatatcaggtttgcagttgcaggcaatacaataaaagggttctcgcagcttaaacttgcaggcgctatgcgggagtacaccgagaaagacttggcctttagcgcagaagaggcggtggcgttagcggaggcagagtctgttcttggagttcctgaagaacagatagagaccttggtgcaagaagttgaggggtggcctgctcttgtagtttttttgttaaagcgtgagttgccggccaagcatatttcagcagtagttgaagtagacaattactttagggatgaaatatttgaggcgattcccgagcgctatcgtgtttttcttgcaaattcttcattgctcgatttcgtgacgcctgatcaatacaattatgtattcaaatgcgtcaatggggtctcatgtattaagtatttaagcactaattacatgttgcttcgccatgtgagcggtgagccagcgcagtttacactgcatccagtactgcgtaattttctacgagaaattacttggactgaaaatcctgctaaaagatcctacctgcttaagcgtgcagctttctggcattggcgtagaggtgaataccagtatgcaatacgaatatccctacgggcgaatgactgtcgctgggcagtcagcatgtctgagagaataattttagatttgtcatttcgtcagggcgaaatagatgcgctgagacagtggctgttagagctgccgaagcaggcctggcacaaaaaacccatagtgcttattagttacgcgtgggtattgtatttcagtcagcaaggcgcgcgagcagagaagttaattaaagacctatcttcacaatccgataaaaaaaataaatggcaagaaaaggaatggctgcagcttgtgcttgcaataggtaaagcaacgaaagatgaaatgctttcgagtgaggagctctgtaataagtggattagtttatttggggattcaaacgcagttggaaaaggggccgcgctaacctgtttggcttttatttttgccagtgagtatagatttgcagagttggagaaggtgctggctcaggcccaagccgtgaataaatttgcaaaacaaaattttgcttttggttggctgtatgtcgcgaggtttcaacaagccctagcaagcggaaaaatgggctgggcgaggcagattataactcaagcacgcacagacagtcgcgcgcagatgatggaatccgagtttacttcgaaaatgtttgacgctctagagcttgagttacattatgaattgcgctgcttggacacctcagaagaaaagctctccaaaattttagagttcatttccaatcacggggtgacagacgtgtttttttccgtatgccgtgctgtgtcagcttggcggcttggaaggagtgacctaaatggctccattgagatattggagtgggcgaaggcgcatgcggttgaaaaaaatctaccaagattggaagttatgagccaaattgagatctatcagcgcttagtctgtcaaggcataacgggcataaataatttaaaaactcttgaagatcataagattttctccggacagcactcagcccccctaaaagcacgcctgctgcttgttcaatcactagtgctttcccgagatcggaactttcatagtgccgcgcacagagcgttattggctattcagcaagcccgtaaaattaacgcgggccagctggaagtccgtggattattgtgtttggccggagcgcaggcaggtgccggtgatttaaaaaaggctcagcttaacattgtttatgcagtggagatagcaaaacagcttcaatgctttcaaacagttcttgatgaagtatgtttaattgagcgaataataccggcttcatgtgaagccttcacagcagttaatttagatcaagcgattggggcttttagtcttccgcgaatagttgagattggaaagtccgcagagaataaagctgacgctttattgacacggaagcagattgctgtcttgaggcttgtaaaagaggggtgctcaaacaaacaaatagcaacaaatatgcatgtcaccgaagatgctataaagtggcatatgaggaaaatatttgccaccttgaatgtagtgaatcgcacgcaagcaacaattgaagctgagcgtcaaggaattatctaatgcaggtcgactctagaggatccccgggtaccgagctcgaattcactggccgtcgttttacagccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgc

SEQ ID NO: 70 - DNA, 5040 bp, Artificial Sequence
  plasmid 4-52
ttctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgccggtagctattgactatccggctgcactagccctgcgtcagatggctctgatccaaggcaaactgccaaaatatctgctggcaccggaagtcagcgccctgcaccattatgttccggatctgcatcgcaggatgctgctggctaccctgtggaacacctacatctgtattaacgaagcgctggcattgaccctgagtgatttttctctggtgccgccctatccctttgtgcagcttgccacgctcaaaggggtttgaggtccaaccgtacgaaaacgtacggtaagaggaaaattatcgtctgaaaaatcgattagtagacaagaaagtccgttaagtgccaattttcgattaaaaagacaccgttttgatggcgttttccaatgtacattatgtttcgatatatcagacagttacttcactaacgtacgttttcgttctattggccttcagaccccatatccttaatgtcctttatttgctggggttatcagatccccccgacacgtttaattaatgctttctccgccggagatcgacgcacagcgttctgtgctctatgatgttatttcttaataatcatccaggtattctctttatcaccatacgtagtgcgagtgtccaccttaacgcagggctttccgtcacagcgcgatatgtcagccagcggggctttcttttgccagaccgcttccatcctctgcatttcagcaatctggctatacccgtcattcataaaccacgtaaatgccgtcacgcaggaagccaggacgaagaatatcgtcagtacaagataaatcgcggatttccacgtatagcgtgacatctcacgacgcatttcatggatcatcgctttcgccgtatcggcagcctgattcagcgcttctgtcgccggtttctgctgtgctaatccggcttgtttcagttctttctcaacctgagtgagcgcggaactcaccgatttcctgacggtgtcagtcatattaccggacgcgctgtccagctcacgaatgaccctgctcagcgtttcactttgctgctgtaattgtgatgaggcggcctgaaactgttctgtcagagaagtaacacgcttttccagcgcctgatgatgcccgataagggcggcaatttgtttaatttcgtcgctcatacaaaatcctgcctatcgtgagaatgaccagcctttatccggcttctgtcgtatctgttcggcgagtcgctgtcgttctttctcctgctgacgctgtttttccgccagacgttcgcgctctctctgcctttccatctcctgatgtatcccctggaactccgccatcgcatcgttaacaagggactgaagatcgatttcttcctgtatatccttcatggcatcactgaccagtgcgttcagcttgtcaggctctttttcaaaatcaaacgttctgccggaatgggattcctgctcaggctctgacttcagctcctgttttagcgtcagagtatccctctcgctgagggcttcccgtaacgaggtagtcacgtcaattacgctgtcacgttcatcacgggactgctgcacctgcctttcagcctccctgcgctcaagaatggcctgtagctgctcagtatcgaatcgctgaacctgacccgcgcccagatgccgctcaggctcacggtcaatgccctgcgccttcagggaacgggaatcaacccggtcagcgtgctgataccgttcaaggtgcttattctggaggtcagcccagcgttccctctgggcaacaaggtattctttgcgttcggtcggtgtttccccgaaacgtgccttttttgcgccaccgcgctccggctctttggtgttagcccgtttaaaatactgctcagggtcacggtgaataccgtcattaatgcgttcagagaacatgatatgggcgtggggctgctcgccaccggctatcgctgctttcggattatggatagcgaactgataggcatggcggtcgccaatttcctgttggacaaaatcgcggacaagctcaagacgttgttcgggttttaactcacgcggcagggcaatctcgatttcacggtaggtacagccgttggcacgttcagacgtgtcagcggctttccagaactcggacggtttatgcgctgcccacgccggcatattgccggactccttgtgctcaaggtcggagtctttttcacgggcatactttccctcacgcgcaatataatcggcatgaggagaggcactgccttttccgccggtttttacgctgagatgataggatgccatcgtgttttatcccgctgaaggcgcgcaccgtttctgaacgaagtgaagaaacgtctaagtgcgccctgataaataaaagagttatcagggattgtagtgggatttgacctcctctgccatcactgagcataatcattccgttagcattcaggaggtaaacagcatgaataaaagcgaaaaacaggaacaatgggcagcagaaagagtgcagtatattcgcggcttaaagtcgccgaatgagcaacagaaacttatgctgatactgacggataaagcagataaaacagcacaggatatcaaaacgctgtccctgctgatgaaggctgaacaggcagcagagaaagcgcaggaagccagagcgaaagtcatgaacctgatacaggcagaaaagcgagccgaagccagagccgcccgtaaagcccgtgaccatgctctgtaccagtctgccggattgcttatcctggcgggtctggttgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttat

SEQ ID NO: 71 - DNA, 58 bp, Artificial Sequence
  primer_o-HM-51
ctcggtacccggggatcctctagagttatcagactcctaacttccatgagagggtacg

SEQ ID NO: 72 - DNA, 43 bp, Artificial Sequence
  primer_o-HM-52
gcgtcaaggaattatctaatgcaggtcgcggcatctccggcca

SEQ ID NO: 73 - DNA, 14946 bp, Artificial Sequence
  plasmid_HM-p-50
gcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccaaaaaagccgccgcacactggaggatgtgtgaggcagcttttctaggacttaacgcttagatcagaacagtccggttgggtttggatcgtaggagaccaacaggttcttggtctgctggtagtggttcagcatcatgaggtggttctcacggccgatgccggactccttgtatccaccgaaagcggagtgcgctgggtagttgtggtactggttgacccaaactcgaccagcctggattgcgcgacctgcacgataaatggtgttttggtcacggctccagacaccagcgccgaggccgtagttggtgtcgtttgcaatacggatggcctcatcgaagtcgctgaaggtagcaacagaaaggactggtccgaagatttcctcgcggaagatcctcatgtcgttggtgccgcggaaaacggttggctcaatgtagtaaccgttctccatgccatcaaccttgttgaccttgccaccagtgagggtttgagcgccttcttctgggccgatcttcaggtaggaggagatcttgtccatctgctcctgggacgcctgagcacccatcatggtttcagtatcaagtgggttacccagcttgatgttctgaactcgcttcacgccaagctcgaggaattcatcagcgatggactcatgaacaagtgcacgggaaggacaggtacaaacttcaccctgattgagggcgaacatcgcgaagccttcaactgccttctctgcgaaggcgtcatcctgtgacagaacatcggagaagaagatggatggggacttaccgccgagctccagggtgacaggaatgatcttgtcggatgcagcgcggttgatcagcttgccgacctcggtggaaccggtgaaagcaatcttgccaatccgattagagccggacagtgcagcgcctgcttcaccgccgagtccgttgacgatgttgaggacgccctctgggatgagatcgccgatgatgttaatcagatacaaaatggatgctggggtctgctcagctggcttcatgacgatcgcgttacctgcagcaagtgccggtgcgagcttccaggtagccatgaggattgggaagttccaaggaatgatctgaccaacaacaccgattggctcgttgaagtggtaagcaacagtgttgtggtcgatctgtgaggaacgatcttcctgagcacggatcgcgccagcaaagtagcggaagtggtcgattgccagtgggatatctgcagcaagagtctcacggactgccttgccgttctcccaggtttctgcaactgcgatttcttccaggtgctcttccatgcggtccgcaatgcggtgcaggatcagagcacgttcagcgacagaagtcttgccccacgcatcagcggctgcatgtgcagcatccagtgcaagctccacgtccgctgcggtgccacgtgcgacctcacagaaaacttcaccagtgacaggtgaaatgttctcaaggtactggccctctaccggtggaacccacttgccaccaatgtagttctcgtagcgcttttcatagttaacgatcgagccttcggttcctggatttgcgtagacagtcattgggtctcctttgggccacctccacctgagcactacttagaaacgaatcgccacacgaccatcgatcttgccgtttcgcatgcggtcaagcacaccattgacctcatcgagggagcactcactcacggttggcttgattagtccgcgtgcaaagaaatcgagcgcttcggccaagtcttggcgggttcccacgagggatccacggatggtcaggcccttgaatacgatgttgaacacggatgctgggaactctcccggtggcagaccgttgaacacaattgttcctgcacgtcgagccatatccagtgcctggccgaatgctgcctcgtgaactgcagtcacaagcacgccgtgtgcgccaccgttggtgtacttctgtacagcttcgcctgaatcttcattacgcgcattcacggtaaattccgcaccgtgcttacgggcaagttccagcttgtcatcggcaatatctaccgcaatgacacgcatgcccatcgccgctgcgtattggactgcgatgtggccaagtccgccgacaccggagatcaccatgaattggcccgggcgggtttcagagactttgagtgccttgtagacagtcacgcctgcacacagaattggtgctgcttcgaggtagtccacgccgtctgggatgcgagcggcgtaacgggtatccaccagcatgtactggccgaaggatccattttgggtgtagccaccatactcagcttcgttgcactgagtttccctgccggtgatgcagtattcgcaggtgccacacgctgaccagagccacgcattgccgacaatatcgccgaccttcacatcgtgttcacctggtccgagctcaacaacttcacctacaccttcgtgtcctggtacgaatggtggttccggctttactggccaatcgccctccaaggcgtggaggtcggtgtggcagatgccggaggtgagtaccttcaccaatgcctggtgtggccctggctttggaaggtcaatatccttcacggtcacgtcatgaccgaatttttcaacaacagcagcggtaaattcttggggtgcagcagtggtcataaaactcactccttctcgcttggattacttggcaaattgcttccaatttttattaaattagtcgctacgagatttaagacgtaattttatgcctaactgagaaagttaagccgcccactctcactctcgacatcttaaacctgagctaatcggacgcttgcgccaactacacctacgggtagtttttgctccgtcgtctgctggaaaaacacgagctggccgcaagcatgccaggtaccgcgagctactcgcgacggctgaaagcaccgaaatgagcgagctatctggtcgattttgacccggtgcccgtcttcaaaatcggcgaaggccgaagtcggccagaaatagcggcctacttcagaccttccctagtaaatattttgcaccaccgatcatgccgactacacttaagtgtagttttaatatttaacaccgtaacctatggtgaaaatttccagtcagctggcgcgagaatagcataatgaaaataataataaataatgatttcccggtcgctaaggtcggagcggatcaaattacgactctagtaagtgccaaagttcatagttgcatatatcggccaagattgagtatcgcggatggagccgctcccagagtatgcctttacagagccccacctggatatgggaaaaccgttgctcttgcgttcgagtggctacgccacagaacagccggacgtcctgcagtgtggctttctttaagagccagttcttacagtgaatttgatatctgcgcagagattattgagcagcttgaaactttcgaaatggtaaaattcagccgtgtgagagagggtgtgagcaagcctgcgctcttgcgagaccttgcatctagtctttggcagagcacctcgaataacgagatagaaacgctagtttgtttggataatattaatcatgacttagacttgccgttgttgcacgcacttatggagtttatgttaaatacaccaaaaaatatcaggtttgcagttgcaggcaatacaataaaagggttctcgcagcttaaacttgcaggcgctatgcgggagtacaccgagaaagacttggcctttagcgcagaagaggcggtggcgttagcggaggcagagtctgttcttggagttcctgaagaacagatagagaccttggtgcaagaagttgaggggtggcctgctcttgtagtttttttgttaaagcgtgagttgccggccaagcatatttcagcagtagttgaagtagacaattactttagggatgaaatatttgaggcgattcccgagcgctatcgtgtttttcttgcaaattcttcattgctcgatttcgtgacgcctgatcaatacaattatgtattcaaatgcgtcaatggggtctcatgtattaagtatttaagcactaattacatgttgcttcgccatgtgagcggtgagccagcgcagtttacactgcatccagtactgcgtaattttctacgagaaattacttggactgaaaatcctgctaaaagatcctacctgcttaagcgtgcagctttctggcattggcgtagaggtgaataccagtatgcaatacgaatatccctacgggcgaatgactgtcgctgggcagtcagcatgtctgagagaataattttagatttgtcatttcgtcagggcgaaatagatgcgctgagacagtggctgttagagctgccgaagcaggcctggcacaaaaaacccatagtgcttattagttacgcgtgggtattgtatttcagtcagcaaggcgcgcgagcagagaagttaattaaagacctatcttcacaatccgataaaaaaaataaatggcaagaaaaggaatggctgcagcttgtgcttgcaataggtaaagcaacgaaagatgaaatgctttcgagtgaggagctctgtaataagtggattagtttatttggggattcaaacgcagttggaaaaggggccgcgctaacctgtttggcttttatttttgccagtgagtatagatttgcagagttggagaaggtgctggctcaggcccaagccgtgaataaatttgcaaaacaaaattttgcttttggttggctgtatgtcgcgaggtttcaacaagccctagcaagcggaaaaatgggctgggcgaggcagattataactcaagcacgcacagacagtcgcgcgcagatgatggaatccgagtttacttcgaaaatgtttgacgctctagagcttgagttacattatgaattgcgctgcttggacacctcagaagaaaagctctccaaaattttagagttcatttccaatcacggggtgacagacgtgtttttttccgtatgccgtgctgtgtcagcttggcggcttggaaggagtgacctaaatggctccattgagatattggagtgggcgaaggcgcatgcggttgaaaaaaatctaccaagattggaagttatgagccaaattgagatctatcagcgcttagtctgtcaaggcataacgggcataaataatttaaaaactcttgaagatcataagattttctccggacagcactcagcccccctaaaagcacgcctgctgcttgttcaatcactagtgctttcccgagatcggaactttcatagtgccgcgcacagagcgttattggctattcagcaagcccgtaaaattaacgcgggccagctggaagtccgtggattattgtgtttggccggagcgcaggcaggtgccggtgatttaaaaaaggctcagcttaacattgtttatgcagtggagatagcaaaacagcttcaatgctttcaaacagttcttgatgaagtatgtttaattgagcgaataataccggcttcatgtgaagccttcacagcagttaatttagatcaagcgattggggcttttagtcttccgcgaatagttgagattggaaagtccgcagagaataaagctgacgctttattgacacggaagcagattgctgtcttgaggcttgtaaaagaggggtgctcaaacaaacaaatagcaacaaatatgcatgtcaccgaagatgctataaagtggcatatgaggaaaatatttgccaccttgaatgtagtgaatcgcacgcaagcaacaattgaagctgagcgtcaaggaattatctaatgcaggtcgcggcatctccggccaggcaaagggaattgcggattgacctagccgaactccttggtaaatcgctgcgtgtttatgcgtccccgagatagttgcggccgactctgcgcttaatgggctcggttggcatgaggggcggctggggtcactcgaggagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagtcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatattcaccaccctgaattgactctcttccgggcgctatcatgccataccgcgaaaggttttgcgccattcgatggcgcgccggcagtgagcgcaacgcaattaatgtaagttagctcactcattaggcaccccaggcttgacactttatgcttccggctcgtataatgtgtggaattgtgagcggataacaataacaatttcacacaggatctaggaaccaaggagagtggcatgcccaccctcgcgccttcaggtcaacttgaaatccaagcgatcggtgatgtctccaccgaagccggagcaatcattacaaacgctgaaatcgcctatcaccgctggggtgaataccgcgtagataaagaaggacgcagcaatgtcgttctcatcgaacacgccctcactggagattccaacgcagccgattggtgggctgacttgctcggtcccggcaaagccatcaacactgatatttactgcgtgatctgtaccaacgtcatcggtggttgcaacggttccaccggacctggctccatgcatccagatggaaatttctggggtaatcgcttccccgccacgtccattcgtgatcaggtaaacgccgaaaaacaattcctcgacgcactcggcatcaccacggtcgccgcagtacttggtggttccatgggtggtgcccgcaccctagagtgggccgcaatgtacccagaaactgttggcgcagctgctgttcttgcagtttctgcacgcgccagcgcctggcaaatcggcattcaatccgcccaaattaaggcgattgaaaacgaccaccactggcacgaaggcaactactacgaatccggctgcaacccagccaccggactcggcgccgcccgacgcatcgcccacctcacctaccgtggcgaactagaaatcgacgaacgcttcggcaccaaagcccaaaagaacgaaaacccactcggtccctaccgcaagcccgaccagcgcttcgccgtggaatcctacttggactaccaagcagacaagctagtacagcgtttcgacgccggctcctacgtcttgctcaccgacgccctcaaccgccacgacattggtcgcgaccgcggaggcctcaacaaggcactcgaatccatcaaagttccagtccttgtcgcaggcgtagataccgatattttgtacccctaccaccagcaagaacacctctccagaaacctgggaaatctactggcaatggcaaaaatcgtatcccctgtcggccacgatgctttcctcaccgaaagccgccaaatggatcgcatcgtgaggaacttcttcagcctcatctccccagacgaagacaacccttcgacctacatcgagttctacatctaataggtatttacgacaaatagacagggatctctaaacaactcacaggcaaccctccggataaacggatccaattgtgagcggataacaattacgagcttcatgcacagtgatcgacgctgttgacaattaatcatcggctcgtataatgtgtggatgtggaattgtgagcgctcacaattccacaacggtttccctctagaaataattttgtttaacaggaggtaaaacatatgcgagtgttgaagttcggcggtacatcagtggcaaatgcagaacgttttctgcgtgttgccgatattctggaaagcaatgccaggcaggggcaggtggccaccgtcctctctgcccccgccaaaatcaccaaccacctggtggcgatgattgaaaaaaccattagcggccaggatgctttacccaatatcagcgatgccgaacgtatttttgccgaacttttgacgggactcgccgccgcccagccggggttcccgctggcgcaattgaaaactttcgtcgatcaggaatttgtccaaataaaacatgtcctgcatggcattagtttgttggggcagtgcccggatagcatcaacgctgcgctgatttgccgtggcgagaaaatgtcgatcgccattatggccggcgtattagaagcgcgcggtcacaacgttactgttatcgatccggtcgaaaaactgctggcagtggggcattacctcgaatctaccgtcgatattgctgagtccacccgccgtattgcggcaagccgcattccggctgatcacatggtgctgatggcaggtttcaccgccggtaatgaaaaaggcgaactggtggtgcttggacgcaacggttccgactactctgctgcggtgctggctgcctgtttacgcgccgattgttgcgagatttggacggacgttgacggggtctatacctgcgacccgcgtcaggtgcccgatgcgaggttgttgaagtcgatgtcctaccaggaagcgatggagctttcctacttcggcgctaaagttcttcacccccgcaccattacccccatcgcccagttccagatcccttgcctgattaaaaataccggaaatcctcaagcaccaggtacgctcattggtgccagccgtgatgaagacgaattaccggtcaagggcatttccaatctgaataacatggcaatgttcagcgtttctggtccggggatgaaagggatggtcggcatggcggcgcgcgtctttgcagcgatgtcacgcgcccgtattttcgtggtgctgattacgcaatcatcttccgaatacagcatcagtttctgcgttccacaaagcgactgtgtgcgagctgaacgggcaatgcaggaagagttctacctggaactgaaagaaggcttactggagccgctggcagtgacggaacggctggccattatctcggtggtaggtgatggtatgcgcaccttgcgtgggatctcggcgaaattctttgccgcactggcccgcgccaatatcaacattgtcgccattgctcagggatcttctgaacgctcaatctctgtcgtggtaaataacgatgatgcgaccactggcgtgcgcgttactcatcagatgctgttcaataccgatcaggttatcgaagtgtttgtgattggcgtcggtggcgttggcggtgcgctgctggagcaactgaagcgtcagcaaagctggctgaagaataaacatatcgacttacgtgtctgcggtgttgccaactcgaaggctctgctcaccaatgtacatggccttaatctggaaaactggcaggaagaactggcgcaagccaaagagccgtttaatctcgggcgcttaattcgcctcgtgaaagaatatcatctgctgaacccggtcattgttgactgcacttccagccaggcagtggcggatcaatatgccgacttcctgcgcgaaggtttccacgttgtcacgccgaacaaaaaggccaacacctcgtcgatggattactaccatcagttgcgttatgcggcggaaaaatcgcggcgtaaattcctctatgacaccaacgttggggctggattaccggttattgagaacctgcaaaatctgctcaatgcaggtgatgaattgatgaagttctccggcattctttctggttcgctttcttatatcttcggcaagttagacgaaggcatgagtttctccgaggcgaccacgctggcgcgggaaatgggttataccgaaccggacccgcgagatgatctttctggtatggatgtggcgcgtaaactattgattctcgctcgtgaaacgggacgtgaactggagctggcggatattgaaattgaacctgtgctgcccgcagagtttaacgccgagggtgatgttgccgcttttatggcgaatctgtcacaactcgacgatctctttgccgcgcgcgtggcgaaggcccgtgatgaaggaaaagttttgcgctatgttggcaatattgatgaagatggcgtctgccgcgtgaagattgccgaagtggatggtaatgatccgctgttcaaagtgaaaaatggcgaaaacgccctggccttctatagccactattatcagccgctgccgttggtactgcgcggatatggtgcgggcaatgacgttacagctgccggtgtctttgctgatctgctacgtaccctctcatggaagttaggagtctgataactctagaggatccccgggtaccgagctcgaattcactggccgtcgttttacagccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgc

SEQ ID NO: 74 - DNA, 4174 bp, Artificial Sequence
  plasmid_HM-p-54
ttctcatgtttgacagcttatcatcgataggcagtgagcgcaacgcaattaatgtaagttagctcactcattaggcaccccaggcttgacactttatgcttccggctcgtataatgtgtggaattgtgagcggataacaatgcgcaatttgccaagtaatccaagcgagaaggagtgagttttatgttcacgggaagtattgtcgcgattgttactccgatggatgaaaaaggtaatgtctgtcgggctagcttgaaaaaactgattgattatcatgtcgccagcggtacttcggcgatcgtttctgttggcaccactggcgagtccgctaccttaaatcatgacgaacatgctgatgtggtgatgatgacgctggagctggctgacgggcgcattccggtgattgccgggactggtgctaacgctactgcgacagccattagcctgacgcagcgcttcaatgacagtggtatcgtcggctgcctgacggtaaccccttactacaatcgtccgtcgcaagaaggtttgtatcagcatttcaaagccatcgctgagcatactgacctgccgcaaattctgtataatgtgccgtcccgtactggctgcgatctgctcccggaaacggtgggccgtctggcgaaagtaaaaaatattatcggaatcaaagaggcaacagggaacttaacgcgtgtaaaccagatcaaagagctggtttcagatgattttgttctgctgagcggcgatgatgcgagcgcgctggacttcatgcaattaggcggtcatggggttatttccgttacggctaacgtcgcagcgcgtgatatggcccagatgtgcaaactggcagcagaagggcattttgccgaggcacgcgttattaatcagcgtctgatgccattacacaacaaactatttgtcgaacccaatccaatcccggtgaaatgggcatgtaaggaactgggtcttgtggcgaccgatacgctgcgcctgccaatgacaccaatcaccgacagtggtcgtgagacggtcagagcggcgcttaagcatgccggtttgctgtaataccggtgtcgacatcgataaggctagctcgagcgcggccgcgcgatcgcacctggtgtttaaacggccggcccctgcaggggcgcgcccccggggggcccccgcggatctctagagtcccaaggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 75 - DNA, 18060 bp, Artificial Sequence
  plasmid_HM-p-48
aaataattttgtttaactttaagaaggagatatacgatgtcagcaaatatggcagtcaaacaggccttgaaggccaatccggtcccgagttccgtggatcctcaggaagtccacaaatggcttcaggatttcacttgggatttcaagggcaagaccgcgaagtatccgaccaagtatgagatggacgtcaatacgcgcgagcagttcaagctgactgccaaggagtacgcgcgcatggagtcgatcaaggaagagcgccagtacggcaccctgctcgatggtctcgaccgtctggatgcgggcaacaaggtgcatccgaaatggggcgaggtgatgaagctggtctccaacttcctcgagaccggcgaatacggcgcaatcgccggttctgctctgctgtgggacacggcccagtcaccggagcagcgcaacggttacctcgctcaggtgatcgacgaaatccgccatgtgaaccagaccgcgtacgtgaattactactacggcaagcactactacgaccccgccggccacaccaacatgcgccagcttcgggcgatcaaccccctgtaccccggtgtcaagcgcgccttcggggagggctttctcgcgggcgatgccgtcgagtcctccatcaacctccaattggtcggcgaagcctgcttcaccaatccgctcatcgtttcgcttaccgaatgggcggccgcgaacggcgatgaaatcacgccgaccgtgttcctgtcgatcgaaaccgatgaactgcgccatatggccaacggctatcagaccatcgtgtcgatcatgaacaatccggagacgatgaagtacctgcaaacggacctcgataacgcattctggacgcagcacaagttcctgacccccttcgtcggggtggcgctcgaatatggttccaagtacaaggtcgagccgtgggccaagtcctggaaccggtgggtgtacgaagactgggctggcatctggctgggccgactgcagcagttcggcgtcaaaacgccaaagtgcctgcccgacgccaagaaagacgccgtttgggcacaccacgatctcgcgctgctggcccttgccctgtggccgctgaccggcatccgcatggaactcccggatagcctggcgatggagtggttcgaggctaattaccccggttggtacaaccattacggcaagatctacgaggagtggcgcgctgccggcttcgaggatccgaagagcggcttctgtggtgcgctctggctgatggagcgtggtcacggcattttcgtcgaccatgcctcgggtttgccgttctgccccagcctcgccaaaagctctatcaagccgcggttcactgaatacaacggcaagcgctacgccttcgccgagccgtatggcgagcgccagtggctgctcgagcccgagcgctacgagttccagaacttcttcgagcagttcgaaggctgggaactctccgatctcgtcaaggcggcaggcggcgtgcgcagcgatggcaaaacgctgatcgcgcagccgcatcttcgcgacaccgacatgtggacgcttgacgatctgaagcggatcaacctgacgatccccgacccgatgaagatcctcaactggcaacccgtcgcccagtgagggttgggccggcgcacgccgccaggcgagagatgcgccggcggggaagacttgccgcccgcctacgagcgggcggacgtgccaacgaacgaacagcactatatggagtagataaatatgtcaacaaacatctttacgcgcgggatggtagacccggagcgccaagcctgcattcaggaggtcgtacccaaggcgccgctggaaaccaagcgtgaccatattcctttcgccaaacgcggctggcgccgactcaccgagtatgaagcggtgatgttgcatgcgcagaattcactggacgccgtcccgggcagccaggaggtgggtgaagtcgtacagaagtggccgggtggcaggccgaactacggcgtcgagtcgaccgcggccctctccagcaactggttccatttccgcgacccgtcgaagcgctggttcatgccctacgtgaagcagaagaacgaggaagggcagacagccgaacgcgcgatgaagagttgggccgagggcggcgacgcggaaatgatgaacgccgcgtggcgggagcacatcctggcccggcactacggcgcgttcgtctataacgagtacggtctgttcagcgcccattcaacaacagtttacggtggcctttctgatctgatcaaaacctggattgccgaggccgcgttcgacaagaacgacgccggtcagatgatccagatgcagcgcgtgctgttgagcaaggtgttccctggcttcgacgccgacctggccgaagccaagcaggcatggactgaggacaagtcatggaaacccgcccgcgagttcgtcgagcacatctgggccgagacctacgactgggtcgagcagctttgggcgatccacgccgtctacgaccatattttcgggcaatttgtccggcgcgaattcttccagcggctcggcggcatccatggagacacgctgacgcccttcattcagaaccaggcgctgacgtatcacctgcaagcccgcgacggcgtaacggcactgtgcttcaaatttctgatcgaagacgagccggtatacgcccaacacaacaggcgctacctgcgggcatggacagggcgctatctcccgcaagtgggccgcgcattgaaggccttcctggcaatctacaaggaggttccggtaaagatcgatggggtgacttgccgggagggcgtgcgcgccagtgtcgaacgcgtggtggacgactgggcggcgcggttcgctgaaccgatcaacttcaagttcaaccgcgcggcgttcatcgacgatgtgctgagtggctactaattcaggggcagaccgaatgtcaaacgtaaatgcataccacgctggcaccaatggtaaagaaggccaggacttcatcgatgactttttgagcgaagagaacagtgctctacccacgagcgaagccgtggttctggccctgatgaagaccgaagagatcgacgcggtcgtcgacgagatgatcaagccgcagatggaggacaacccgaccatcgccgtcgaggaccgcggtggatactggtggatcaaggccaacggaaagatcgtcatcgattgtgacgaagccaccgaactgctgggcaagaagtatacggtgtacgatttgctggtcaacgtcagcaccaccgttgggcgggcgatgaccctcggcaatcagttcatcatcaccaacgagctccttggtctcgaaaccaaggtcgaaagcgtttactgaggaggatagacgacatgtcgaaacaggtttggtacaacacaccggtgcgcgacgagtggatcgagaagatcactgcgatcaggacagcgcgcgaaggcaccgacatgctcgcccgcttccgcgcggagcatacggggccggatcgcacgacgtacgatctcaagaaggaatacaattggatcgagtcgcgcatcgagatgcgggtcagccagcttcatgccgaggccacggcctcggacgaagacctcctcaccaagacgatcgacggccgctgcgcaaaggaagtcgcagcggagtggctgaaaaaggctgcggacatcgactgtcactacgagatggagcggctttgtgtcgccttccgcaaggcgtgcaaaccgccgatgatgcccatcaacttcttcgcgccggcagagaaggagttggtggcgaagctcatgaagctgagggcgcccacgtatctgactacctccctcgacgagttgcgcgaggcacggggtgtaacgatgatttccgtgcagtaagcggccggacgttctgagccggatcctccccgacaacggggaggactggtcaaaatagccatcaagactgaaaggagcagggaatgccgaaatacatcatcgaacgttcgattccgggggcagggaagctgacggagcgggacctcgcgtcggtgtcgcagaaatcctgtggaatcctccgctccatggggccacaaatccaatgggtcaaaagctacgtcaccgacgacaagctctactgcatctatatcgctcccgatgaggcctccatccgcgagcatgcaagatcgggagactttcccgccgacaaggtgtcccgcatacatcggatgatcgacccgacttgcgcggaatcggcataatcgagccgtcctcggaggaccacgacttcccgagggcctccggacgatggatcgcggcggacggaaacctgcgtggccgcggacgcggggtggccacaagatcgtaacgtaaagtactctaatccgagagcgcggtcgctttcacccgatcgaggtggaagtgactgggcgagggggagatatgaaagaagcgccggcaatacccgatctgcccggtttgcccgagactgtcggcgaaccgacgctggtcctggaagaagatggcttccgggttttcgccacagagctgacgatcatgtggcgatgggacatctacaacggcgatgcgcacgttcataccggatgcgcccagcacccggagagctgcgtcgtcgcggcgagatccaaaattcgattcttacggcgccccactgtggccatgctactgggcggtgaaggtcaatgagagggggttcgagacgccaactgatggacgcgacaccctagtaggagactgaaggtatatgctgatgcaacaatataaaatagtcgcccgcttcgaagacggcgtgacatacgagtacgattgcggcgaggacgaaaacctgctcgccgcggcgttgcgccagaacgtccgtttgctgtgtcaatgcagaaaggcgttttgcggcagttgcaaggccttgtgcagcgagggcgactatgaactgggcgatcatatcaacgttcaggtcttgccgcctgacgaagaagaggacggggtggtggtcacctgcgataccttcccacgcagcgatctggtccttgagtttccgtataccagcgaccgtctcggtaccgttacggctaccgaggcgaagacgagcgtcgtcagcgtggagagactctctagcaccgtttaccgtctggtgctgcaggcactcgacgccgaaggaatgcctgcacggttcgacttcgttcccgggcagtatgtcgaaatcagtacagccgattcgctcgagaccagggcgttttcgctggcgaaccttccaaacgacgccggattgctcgagttcttgatccgactcgtccccggcggatactatgcggcctatctggagcaacgcgcagcggcagggcagacgatcaacgtgaagggaccgttcggcgagttcgtgctgcgtgaacacgagttggtggaggacttcacgctgccagcggacagcccagcgagaggtgggacgattgcgttcctggcgggtagcacggggctggcgcctctcgcgagcatgctgcgcgagctcgggcggcgaggattcaacggcgagtgtcacctcttcttcggcatgcaggacaccgcaacgatgttctacgagaaggaactccgggacatcaagcgaacgcttccggggctcacgctgcatctcgccctgatggtcccttctgccgaatgggaaggctaccgtggtaacgccgtagctgcgttcaaagaacatttcgccgccagtagccagatacccgagaacgtttacctgtgcgggcccggaccgatgatcgcggccgcgctcggcgcttgtcgcgagcttggaattcccgacaatagagttcacagggaagaattcgtagcgagcggcggttgaacggcgcttgaagaaaccggggcgcgacgggcagcgtttacgagaccatgagtcaagaagaaagagattttctggccggtgcagcggcaacactcggcgagagcgtcggacaactggagcgccttgcccaagaattaagtgaaaagctgggcgatgagcgtgtcaacattgttcggggtttgctcgaacaggtttatgaccgcgacgacgaagcggaagtgcgtgcatccctctgctatacggagagaaagctgttgtgggcgtgggtccgtttgaagagactgaagggactcaggctcagtattggtcgtggttcgatgagaaacattatatgagaagcagaacgtgatcagtttaaattgcaagaagacaaccacagggttaactgcgcacttggccctggtccgcggaatgaaggcgttggcggaattggttggaacaacgctggggccccagggccgccacgtcatgcttgcgcatcgggccggattggctccgcacgtcagcaaggacggcgtcgaggtcgcacgtcacctgtcgttgccggacagcgaggaagaacttggtgtccggttgctgcgcaacgcagccgtcgcagtgtccgagtcctttggcgacggcacctccactgcaaccgtttttacggcggatctggccgtgcgggcgctcaaactgatcggtgccggggcggataccctggaggtccgtcgaggtctgggtttggctgcctatgccgcgttggtcgccctgaacgacatggcccggcgcgccgaccggggaatgctcaccgccgttgcccaaaccgctgccaacggcgaccgtcgcgtggcggatctcctcgtggaggccttcgaaagggtcggggctgaaggcacgatcgaagtagaaatgggtaattcggtcgaggacgtccttgaagtcgcgcagggcagttatttcgacacggtgccgctcgtcacggccttgttgccgccaacagggcaggtcgagttcgcccggccacttatccttttccattgcgacgcgatcgagacggcggacgagattcttccggcgctggagctagctcgcagttcacggagacccttgctgattctggccgactcggtgggtatcgacgtcgagacgctgctcgtccggaatcaaaatgaaggcaccttggctgttgcggtcgtcagggcgccaatgtatggcgatacgcgtcgcgaggccctgcttgacctgacgagcaaattcggggggacggcgtttggaagggagggattcgtcgaattcgcactcaggtctttgggatcactgtcggaaggtgacttggggcaggcggacgaagcaatcctagaggcggatggtgtgactctgagaggcgccggaaacaatccgtctgcattggaagaccgaatcgcgctggtgcgtgctgaactcgatcgcggcgacgtttccgtgggcgactccccatcggcaaagctcgactacatcgagaagcgaaaggagcggctgaagttgctggctgcaggtagcgccaagctgcatatcggggggccgacggacgttgagatcaagacgcgtttgccgctcgccgagaacgctcatcgggcgctcttggcggccgcgaagtcgggagtgctgcccgggggcggcgtcgcaatgattcgtgcggctgaaaaggtgcagcaagagatggggcgacttgaaggcgacgttgcttctggggcatccattttcctgcagtcgcttgacacacccatccggtggattgcacgaaatgcggggctgcgcccggatgaagtgctcgctcgaaccctggcgaacgagtcggacttttacggactcaacgccatgactggccgatatggcgacttggctgaagacggggttctcgacgccctcgatatggtcaccgacgtgatacgcgtcgcagtcagcgtcgtcgggtcgatgttgggcgtcggcgccctggtgactcgcgcatcgcccaagcccgcgccagagcgcttcaagggcacggagcgggtacacgacaagttgatgcgcgagggcgggttcgatgaatgaaatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttattagataattccttgacgcgtttaaacggcagtgagcgcaacgcaattaatgtaagttagctcactcattaggcaccccaggcttgacactttatgcttccggctcgtataatgtgtggaattgtgagcggataacaatgcgcaatttgccaagtaatccaagcgagaaggagtgagttttatgaccactgctgcaccccaagaatttaccgctgctgttgttgaaaaattcggtcatgacgtgaccgtgaaggatattgaccttccaaagccagggccacaccaggcattggtgaaggtactcacctccggcatctgccacaccgacctccacgccttggagggcgattggccagtaaagccggaaccaccattcgtaccaggacacgaaggtgtaggtgaagttgttgagctcggaccaggtgaacacgatgtgaaggtcggcgatattgtcggcaatgcgtggctctggtcagcgtgtggcacctgcgaatactgcatcaccggcagggaaactcagtgcaacgaagctgagtatggtggctacacccaaaatggatccttcggccagtacatgctggtggatacccgttacgccgctcgcatcccagacggcgtggactacctcgaagcagcaccaattctgtgtgcaggcgtgactgtctacaaggcactcaaagtctctgaaacccgcccgggccaattcatggtgatctccggtgtcggcggacttggccacatcgcagtccaatacgcagcggcgatgggcatgcgtgtcattgcggtagatattgccgatgacaagctggaacttgcccgtaagcacggtgcggaatttaccgtgaatgcgcgtaatgaagattcaggcgaagctgtacagaagtacaccaacggtggcgcacacggcgtgcttgtgactgcagttcacgaggcagcattcggccaggcactggatatggctcgacgtgcaggaacaattgtgttcaacggtctgccaccgggagagttcccagcatccgtgttcaacatcgtattcaagggcctgaccatccgtggatccctcgtgggaacccgccaagacttggccgaagcgctcgatttctttgcacgcggactaatcaagccaaccgtgagtgagtgctccctcgatgaggtcaatggtgtgcttgaccgcatgcgaaacggcaagatcgatggtcgtgtggcgattcgtttctaagtagtgctcaggtggaggtggcccaaaggagacccaatgactgtctacgcaaatccaggaaccgaaggctcgatcgttaactatgaaaagcgctacgagaactacattggtggcaagtgggttccaccggtagagggccagtaccttgagaacatttcacctgtcactggtgaagttttctgtgaggtcgcacgtggcaccgcagcggacgtggagcttgcactggatgctgcacatgcagccgctgatgcgtggggcaagacttctgtcgctgaacgtgctctgatcctgcaccgcattgcggaccgcatggaagagcacctggaagaaatcgcagttgcagaaacctgggagaacggcaaggcagtccgtgagactcttgctgcagatatcccactggcaatcgaccacttccgctactttgctggcgcgatccgtgctcaggaagatcgttcctcacagatcgaccacaacactgttgcttaccacttcaacgagccaatcggtgttgttggtcagatcattccttggaacttcccaatcctcatggctacctggaagctcgcaccggcacttgctgcaggtaacgcgatcgtcatgaagccagctgagcagaccccagcatccattttgtatctgattaacatcatcggcgatctcatcccagagggcgtcctcaacatcgtcaacggactcggcggtgaagcaggcgctgcactgtccggctctaatcggattggcaagattgctttcaccggttccaccgaggtcggcaagctgatcaaccgcgctgcatccgacaagatcattcctgtcaccctggagctcggcggtaagtccccatccatcttcttctccgatgttctgtcacaggatgacgccttcgcagagaaggcagttgaaggcttcgcgatgttcgccctcaatcagggtgaagtttgtacctgtccttcccgtgcacttgttcatgagtccatcgctgatgaattcctcgagcttggcgtgaagcgagttcagaacatcaagctgggtaacccacttgatactgaaaccatgatgggtgctcaggcgtcccaggagcagatggacaagatctcctcctacctgaagatcggcccagaagaaggcgctcaaaccctcactggtggcaaggtcaacaaggttgatggcatggagaacggttactacattgagccaaccgttttccgcggcaccaacgacatgaggatcttccgcgaggaaatcttcggaccagtcctttctgttgctaccttcagcgacttcgatgaggccatccgtattgcaaacgacaccaactacggcctcggcgctggtgtctggagccgtgaccaaaacaccatttatcgtgcaggtcgcgcaatccaggctggtcgagtttgggtcaaccagtaccacaactacccagcgcactccgctttcggtggatacaaggagtccggcatcggccgtgagaaccacctcatgatgctgaaccactaccagcagaccaagaacctgttggtctcctacgatccaaacccaaccggactgttctgatctaagcgttaagtcctagaaaagctgcctcacacatcctccagtgtgcggcggcttttgctatttaaatttagataattccttgacgctcagcttcaattgttgcttgcgtgcgattcactacattcaaggtggcaaatattttcctcatatgccactttatagcatcttcggtgacatgcatatttgttgctatttgtttgtttgagcacccctcttttacaagcctcaagacagcaatctgcttccgtgtcaataaagcgtcagctttattctctgcggactttccaatctcaactattcgcggaagactaaaagccccaatcgcttgatctaaattaactgctgtgaaggcttcacatgaagccggtattattcgctcaattaaacatacttcatcaagaactgtttgaaagcattgaagctgttttgctatctccactgcataaacaatgttaagctgagccttttttaaatcaccggcacctgcctgcgctccggccaaacacaataatccacggacttccagctggcccgcgttaattttacgggcttgctgaatagccaataacgctctgtgcgcggcactatgaaagttccgatctcgggaaagcactagtgattgaacaagcagcaggcgtgcttttaggggggctgagtgctgtccggagaaaatcttatgatcttcaagagtttttaaattatttatgcccgttatgccttgacagactaagcgctgatagatctcaatttggctcataacttccaatcttggtagatttttttcaaccgcatgcgccttcgcccactccaatatctcaatggagccatttaggtcactccttccaagccgccaagctgacacagcacggcatacggaaaaaaacacgtctgtcaccccgtgattggaaatgaactctaaaattttggagagcttttcttctgaggtgtccaagcagcgcaattcataatgtaactcaagctctagagcgtcaaacattttcgaagtaaactcggattccatcatctgcgcgcgactgtctgtgcgtgcttgagttataatctgcctcgcccagcccatttttccgcttgctagggcttgttgaaacctcgcgacatacagccaaccaaaagcaaaattttgttttgcaaatttattcacggcttgggcctgagccagcaccttctccaactctgcaaatctatactcactggcaaaaataaaagccaaacaggttagcgcggccccttttccaactgcgtttgaatccccaaataaactaatccacttattacagagctcctcactcgaaagcatttcatctttcgttgctttacctattgcaagcacaagctgcagccattccttttcttgccatttattttttttatcggattgtgaagataggtctttaattaacttctctgctcgcgcgccttgctgactgaaatacaatacccacgcgtaactaataagcactatgggttttttgtgccaggcctgcttcggcagctctaacagccactgtctcagcgcatctatttcgccctgacgaaatgacaaatctaaaattattctctcagacatgctgactgcccagcgacagtcattcgcccgtagggatattcgtattgcatactggtattcacctctacgccaatgccagaaagctgcacgcttaagcaggtaggatcttttagcaggattttcagtccaagtaatttctcgtagaaaattacgcagtactggatgcagtgtaaactgcgctggctcaccgctcacatggcgaagcaacatgtaattagtgcttaaatacttaatacatgagaccccattgacgcatttgaatacataattgtattgatcaggcgtcacgaaatcgagcaatgaagaatttgcaagaaaaacacgatagcgctcgggaatcgcctcaaatatttcatccctaaagtaattgtctacttcaactactgctgaaatatgcttggccggcaactcacgctttaacaaaaaaactacaagagcaggccacccctcaacttcttgcaccaaggtctctatctgttcttcaggaactccaagaacagactctgcctccgctaacgccaccgcctcttctgcgctaaaggccaagtctttctcggtgtactcccgcatagcgcctgcaagtttaagctgcgagaacccttttattgtattgcctgcaactgcaaacctgatattttttggtgtatttaacataaactccataagtgcgtgcaacaacggcaagtctaagtcatgattaatattatccaaacaaactagcgtttctatctcgttattcgaggtgctctgccaaagactagatgcaaggtctcgcaagagcgcaggcttgctcacaccctctctcacacggctgaattttaccatttcgaaagtttcaagctgctcaataatctctgcgcagatatcaaattcactgtaagaactggctcttaaagaaagccacactgcaggacgtccggctgttctgtggcgtagccactcgaacgcaagagcaacggttttcccatatccaggtggggctctgtaaaggcatactctgggagcggctccatccgcgatactcaatcttggccgatatatgcaactatgaactttggcacttactagagtcgtaatttgatccgctccgaccttagcgaccgggaaatcattatttattattattttcattatgctattctcgcgccagctgactggaaattttcaccataggttacggtgttaaatattaaaactacacttaagtgtagtcggcatgatcggtggtgcaaaatatttactagggaaggtctgaagtaggccgctatttctggccgacttcggccttcgccgattttgaagacgggcaccgggtcaaaatcgaccagatagctcgctcatttcggtgctttcagccgtcgcgagtagctcgcggtacctggcatgcttgcggccagctcgtgtttttccagcagacgacggagcaaaaactacccgtaggtgtagttggcgcaagcgtccgattagctcaggtttaagatgtcgagagtgagagtgggcggcttaactttctcagttaggcataaaattacgtcttaaatctcgtagcgactaatttaataaaaatt

SEQ ID NO: 76 - DNA, 879 bp, Escherichia coli
atgttcacgggaagtattgtcgcgattgttactccgatggatgaaaaaggtaatgtctgtcgggctagcttgaaaaaactgattgattatcatgtcgccagcggtacttcggcgatcgtttctgttggcaccactggcgagtccgctaccttaaatcatgacgaacatgctgatgtggtgatgatgacgctggatctggctgatgggcgcattccggtaattgccgggaccggcgctaacgctactgcggaagccattagcctgacgcagcgcttcaatgacagtggtatcgtcggctgcctgacggtaaccccttactacaatcgtccgtcgcaagaaggtttgtatcagcatttcaaagccatcgctgagcatactgacctgccgcaaattctgtataatgtgccgtcccgtactggctgcgatctgctcccggaaacggtgggccgtctggcgaaagtaaaaaatattatcggaatcaaagaggcaacagggaacttaacgcgtgtaaaccagatcaaagagctggtttcagatgattttgttctgctgagcggcgatgatgcgagcgcgctggacttcatgcaattgggcggtcatggggttatttccgttacggctaacgtcgcagcgcgtgatatggcccagatgtgcaaactggcagcagaagggcattttgccgaggcacgcgttattaatcagcgtctgatgccattacacaacaaactatttgtcgaacccaatccaatcccggtgaaatgggcatgtaaggaactgggtcttgtggcgaccgatacgctgcgcctgccaatgacaccaatcaccgacagtggtcgtgagacggtcagagcggcgcttaagcatgccggtttgctgtaa

SEQ ID NO: 77 - DNA, 2749 bp, Artificial Sequence
  lacUV5 promotor region
ggcagtgagcgcaacgcaattaatgtaagttagctcactcattaggcaccccaggcttgacactttatgcttccggctcgtataatgtgtggaattgtgagcggataacaatgcgcaatttgccaagtaatccaagcgagaaggagtgagttttatgaccactgctgcaccccaagaatttaccgctgctgttgttgaaaaattcggtcatgacgtgaccgtgaaggatattgaccttccaaagccagggccacaccaggcattggtgaaggtactcacctccggcatctgccacaccgacctccacgccttggagggcgattggccagtaaagccggaaccaccattcgtaccaggacacgaaggtgtaggtgaagttgttgagctcggaccaggtgaacacgatgtgaaggtcggcgatattgtcggcaatgcgtggctctggtcagcgtgtggcacctgcgaatactgcatcaccggcagggaaactcagtgcaacgaagctgagtatggtggctacacccaaaatggatccttcggccagtacatgctggtggatacccgttacgccgctcgcatcccagacggcgtggactacctcgaagcagcaccaattctgtgtgcaggcgtgactgtctacaaggcactcaaagtctctgaaacccgcccgggccaattcatggtgatctccggtgtcggcggacttggccacatcgcagtccaatacgcagcggcgatgggcatgcgtgtcattgcggtagatattgccgatgacaagctggaacttgcccgtaagcacggtgcggaatttaccgtgaatgcgcgtaatgaagattcaggcgaagctgtacagaagtacaccaacggtggcgcacacggcgtgcttgtgactgcagttcacgaggcagcattcggccaggcactggatatggctcgacgtgcaggaacaattgtgttcaacggtctgccaccgggagagttcccagcatccgtgttcaacatcgtattcaagggcctgaccatccgtggatccctcgtgggaacccgccaagacttggccgaagcgctcgatttctttgcacgcggactaatcaagccaaccgtgagtgagtgctccctcgatgaggtcaatggtgtgcttgaccgcatgcgaaacggcaagatcgatggtcgtgtggcgattcgtttctaagtagtgctcaggtggaggtggcccaaaggagacccaatgactgtctacgcaaatccaggaaccgaaggctcgatcgttaactatgaaaagcgctacgagaactacattggtggcaagtgggttccaccggtagagggccagtaccttgagaacatttcacctgtcactggtgaagttttctgtgaggtcgcacgtggcaccgcagcggacgtggagcttgcactggatgctgcacatgcagccgctgatgcgtggggcaagacttctgtcgctgaacgtgctctgatcctgcaccgcattgcggaccgcatggaagagcacctggaagaaatcgcagttgcagaaacctgggagaacggcaaggcagtccgtgagactcttgctgcagatatcccactggcaatcgaccacttccgctactttgctggcgcgatccgtgctcaggaagatcgttcctcacagatcgaccacaacactgttgcttaccacttcaacgagccaatcggtgttgttggtcagatcattccttggaacttcccaatcctcatggctacctggaagctcgcaccggcacttgctgcaggtaacgcgatcgtcatgaagccagctgagcagaccccagcatccattttgtatctgattaacatcatcggcgatctcatcccagagggcgtcctcaacatcgtcaacggactcggcggtgaagcaggcgctgcactgtccggctctaatcggattggcaagattgctttcaccggttccaccgaggtcggcaagctgatcaaccgcgctgcatccgacaagatcattcctgtcaccctggagctcggcggtaagtccccatccatcttcttctccgatgttctgtcacaggatgacgccttcgcagagaaggcagttgaaggcttcgcgatgttcgccctcaatcagggtgaagtttgtacctgtccttcccgtgcacttgttcatgagtccatcgctgatgaattcctcgagcttggcgtgaagcgagttcagaacatcaagctgggtaacccacttgatactgaaaccatgatgggtgctcaggcgtcccaggagcagatggacaagatctcctcctacctgaagatcggcccagaagaaggcgctcaaaccctcactggtggcaaggtcaacaaggttgatggcatggagaacggttactacattgagccaaccgttttccgcggcaccaacgacatgaggatcttccgcgaggaaatcttcggaccagtcctttctgttgctaccttcagcgacttcgatgaggccatccgtattgcaaacgacaccaactacggcctcggcgctggtgtctggagccgtgaccaaaacaccatttatcgtgcaggtcgcgcaatccaggctggtcgagtttgggtcaaccagtaccacaactacccagcgcactccgctttcggtggatacaaggagtccggcatcggccgtgagaaccacctcatgatgctgaaccactaccagcagaccaagaacctgttggtctcctacgatccaaacccaaccggactgttctga

SEQ ID NO: 78 - protein, 309 aa, Escherichia coli [sequence omitted]

SEQ ID NO: 79 - protein, 421 aa, Corynebacterium glutamicum
MALVVQKYGGSSLESAERIRNVAERIVATKKAGNDVVVVCSAMGDTTDELLELAAAVNPVPPAREMDMLLTAGERISNALVAMAIESLGAEAQSFTGSQAGVLTTERHGNARIVDVTPGRVREALDEGKICIVAGFQGVNKETRDVTTLGRGGSDTTAVALAAALNADVCEIYSDVDGVYTADPRIVPNAQKLEKLSFEEMLELAAVGSKILVLRSVEYARAFNVPLRVRSSYSNDPGTLIAGSMEDIPVEEAVLTGVATDKSEAKVTVLGISDKPGEAAKVFRALADAEINIDMVLQNVSSVEDGTTDITFTCPRSDGRRAMEILKKLQVQGNWTNVLYDDQVGKVSLVGAGMKSHPGVTAEFMEALRDVNVNIELISTSEIRISVLIREDDLDAAARALHEQFQLGGEDEAVVYAGTGR

SEQ ID NO: 80 - protein, 810 aa, Escherichia coli
MSVIAQAGAKGRQLHKFGGSSLADVKCYLRVAGIMAEYSQPDDMMVVSAAGSTTNQLINWLKLSQTDRLSAHQVQQTLRRYQCDLISGLLPAEEADSLISAFVSDLERLAALLDSGINDAVYAEVVGHGEVWSARLMSAVLNQQGLPAAWLDAREFLRAERAAQPQVDEGLSYPLLQQLLVQHPGKRLVVTGFISRNNAGETVLLGRNGSDYSATQIGALAGVSRVTIWSDVAGVYSADPRKVKDACLLPLLRLDEASELARLAAPVLHARTLQPVSGSEIDLQLRCSYTPDQGSTRIERVLASGTGARIVTSHDDVCLIEFQVPASQDFKLAHKEIDQILKRAQVRPLAVGVHNDRQLLQFCYTSEVADSALKILDEAGLPGELRLRQGLALVAMVGAGVTRNPLHCHRFWQQLKGQPVEFTWQSDDGISLVAVLRTGPTESLIQGLHQSVFRAEKRIGLVLFGKGNIGSRWLELFAREQSTLSARTGFEFVLAGVVDSRRSLLSYDGLDASRALAFFNDEAVEQDEESLFLWMRAHPYDDLVVLDVTASQQLADQYLDFASHGFHVISANKLAGASDSNKYRQIHDAFEKTGRHWLYNATVGAGLPINHTVRDLIDSGDTILSISGIFSGTLSWLFLQFDGSVPFTELVDQAWQQGLTEPDPRDDLSGKDVMRKLVILAREAGYNIEPDQVRVESLVPAHCEGGSIDHFFENGDELNEQMVQRLEAAREMGLVLRYVARFDANGKARVGVEAVREDHPLASLLPCDNVFAIESRWYRDNPLVIRGPGAGRDVTAGAIQSDINRLAQLL

SEQ ID NO: 81 - protein, 309 aa, Corynebacterium glutamicum
MAIELNVGRKVTVTVPGSSANLGPGFDTLGLALSVYDTVEVEIIPSGLEVEVFGEGQGEVPLDGSHLVVKAIRAGLKAADAEVPGLRVVCHNNIPQSRGLGSSAAAAVAGVAAANGLADFPLTQEQIVQLSSAFEGHPDNAAASVLGGAVVSWTNLSIDGKSQPQYAAVPLEVQDNIRATALVPNFHASTEAVRRVLPTEVTHIDARFNVSRVAVMIVALQQRPDLLWEGTRDRLHQPYRAEVLPITSEWVNRLRNRGYAAYLSGAGPTAMVLSTEPIPDKVLEDARESGIKVLELEVAGPVKVEVNQP

SEQ ID NO: 82 - protein, 344 aa, Corynebacterium glutamicum
MTTIAVVGATGQVGQVMRTLLEERNFPADTVRFFASPRSAGRKIEFRGTEIEVEDITQATEESLKDIDVALFSAGGTASKQYAPLFAAAGATVVDNSSAWRKDDEVPLIVSEVNPSDKDSLVKGIIANPNCTTMAAMPVLKPLHDAAGLVKLHVSSYQAVSGSGLAGVETLAKQVAAVGDHNVEFVHDGQAADAGDVGPYVSPIAYNVLPFAGNLVDDGTFETDEEQKLRNESRKILGLPDLKVSGTCVRVPVFTGHTLTIHAEFDKAITVDQAQEILGAASGVKLVDVPTPLAAAGIDESLVGRIRQDSTVDDNRGLVLVVSGDNLRKGAALNTIQIAELLVK

SEQ ID NO: 83 - protein, 481 aa, Corynebacterium glutamicum
MDYISTRDASRTPARFSDILLGGLAPDGGLYLPATYPQLDDAQLSKWREVLANEGYAALAAEVISLFVDDIPVEDIKAITARAYTYPKFNSEDIVPVTELEDNIYLGHLSEGPTAAFKDMAMQLLGELFEYELRRRNETINILGATSGDTGSSAEYAMRGREGIRVFMLTPAGRMTPFQQAQMFGLDDPNIFNIALDGVFDDCQDVVKAVSADAEFKKDNRIGAVNSINWARLMAQVVYYVSSWIRTTTSNDQKVSFSVPTGNFGDICAGHIARQMGLPIDRLIVATNENDVLDEFFRTGDYRVRSSADTHETSSPSMDISRASNFERFIFDLLGRDATRVNDLFGTQVRQGGFSLADDANFEKAAAEYGFASGRSTHADRVATIADVHSRLDVLIDPHTADGVHVARQWRDEVNTPIIVLETALPVKFADTIVEAIGEAPQTPERFAAIMDAPFKVSDLPNDTDAVKQYIVDAIANTSVK

SEQ ID NO: 84 - protein, 443 aa, Escherichia coli
MSTSDSIVSSQTKQSSWRKSDTTWTLGLFGTAIGAGVLFFPIRAGFGGLIPILLMLVLAYPIAFYCHRALARLCLSGSNPSGNITETVEEHFGKTGGVVITFLYFFAICPLLWIYGVTITNTFMTFWENQLGFAPLNRGFVALFLLLLMAFVIWFGKDLMVKVMSYLVWPFIASLVLISLSLIPYWNSAVIDQVDLGSLSLTGHDGILITVWLGISIMVFSFNFSPIVSSFVVSKREEYEKDFGRDFTERKCSQIISRASMLMVAVVMFFAFSCLFTLSPANMAEAKAQNIPVLSYLANHFASMTGTKTTFAITLEYAASIIALVAIFKSFFGHYLGTLEGLNGLVLKFGYKGDKTKVSLGKLNTISMIFIMGSTWVVAYANPNILDLIEAMGAPIIASLLCLLPMYAIRKAPSLAKYRGRLDNVFVTVIGLLTILNIVYKLF

SEQ ID NO: 85 - protein, 295 aa, Escherichia coli
MPGSLRKMPVWLPIVILLVAMASIQGGASLAKSLFPLVGAPGVTALRLALGTLILIAFFKPWRLRFAKEQRLPLLFYGVSLGGMNYLFYLSIQTVPLGIAVALEFTGPLAVALFSSRRPVDFVWVVLAVLGLWFLLPLGQDVSHVDLTGCALALGAGACWAIYILSGQRAGAEHGPATVAIGSLIAALIFVPIGALQAGEALWHWSVIPLGLAVAILSTALPYSLEMIALTRLPTRTFGTLMSMEPALAAVSGMIFLGETLTPIQLLALGAIIAASMGSTLTVRKESKIKELDIN

SEQ ID NO: 86 - protein, 206 aa, Escherichia coli
MTLEWWFAYLLTSIILSLSPGSGAINTMTTSLNHGYRGAVASIAGLQTGLAIHIVLVGVGLGTLFSRSVIAFEVLKWAGAAYLIWLGIQQWRAAGAIDLKSLASTQSRRHLFQRAVFVNLTNPKSIVFLAALFPQFIMPQQPQLMQYIVLGVTTIVVDIIVMIGYATLAQRIALWIKGPKQMKALNKIFGSLFMLVGALLASARHA

SEQ ID NO: 87 - protein, 384 aa, Escherichia coli
MAKHLFTSESVSEGHPDKIADQISDAVLDAILEQDPKARVACETYVKTGMVLVGGEITTSAWVDIEEITRNTVREIGYVHSDMGFDANSCAVLSAIGKQSPDINQGVDRADPLEQGAGDQGLMFGYATNETDVLMPAPITYAHRLVQRQAEVRKNGTLPWLRPDAKSQVTFQYDDGKIVGIDAVVLSTQHSEEIDQKSLQEAVMEEIIKPILPAEWLTSATKFFINPTGRFVIGGPMGDCGLTGRKIIVDTYGGMARHGGGAFSGKDPSKVDRSAAYAARYVAKNIVAAGLADRCEIQVSYAIGVAEPTSIMVETFGTEKVPSEQLTLLVREFFDLRPYGLIQMLDLLHPIYKETAAYGHFGREHFPWEKTDKAQLLRDAAGLK

SEQ ID NO: 88 - protein, 652 aa, Escherichia coli
MSQIHKHTIPANIADRCLINPQQYEAMYQQSINVPDTFWGEQGKILDWIKPYQKVKNTSFAPGNVSIKWYEDGTLNLAANCLDRHLQENGDRTAIIWEGDDASQSKHISYKELHRDVCRFANTLLELGIKKGDVVAIYMPMVPEAAVAMLACARIGAVHSVIFGGFSPEAVAGRIIDSNSRLVITSDEGVRAGRSIPLKKNVDDALKNPNVTSVEHVVVLKRTGGKIDWQEGRDLWWHDLVEQASDQHQAEEMNAEDPLFILYTSGSTGKPKGVLHTTGGYLVYAALTFKYVFDYHPGDIYWCTADVGWVTGHSYLLYGPLACGATTLMFEGVPNWPTPARMAQVVDKHQVNILYTAPTAIRALMAEGDKAIEGTDRSSLRILGSVGEPINPEAWEWYWKKIGNEKCPVVDTWWQTETGGFMITPLPGATELKAGSATRPFFGVQPALVDNEGNPLEGATEGSLVITDSWPGQARTLFGDHERFEQTYFSTFKNMYFSGDGARRDEDGYYWITGRVDDVLNVSGHRLGTAEIESALVAHPKIAEAAVVGIPHNIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

SEQ ID NO: 89 - protein, 400 aa, Escherichia coli; identical to SEQ ID NO: 23

SEQ ID NO: 90 - protein, 714 aa, Escherichia coli
MSRIIMLIPTGTSVGLTSVSLGVIRAMERKGVRLSVFKPIAQPRTGGDAPDQTTTIVRANSSTTTAAEPLKMSYVEGLLSSNQKDVLMEEIVANYHANTKDAEVVLVEGLVPTRKHQFAQSLNYEIAKTLNAEIVFVMSQGTDTPEQLKERIELTRNSFGGAKNTNITGVIVNKLNAPVDEQGRTRPDLSEIFDDSSKAKVNNVDPAKLQESSPLPVLGAVPWSFDLIATRAIDMARHLNATIINEGDINTRRVKSVTFCARSIPHMLEHFRAGSLLVTSADRPDVLVAACLAAMNGVEIGALLLTGGYEMDARISKLCERAFATGLPVFMVNTNTWQTSLSLQSFNLEVPVDDHERIEKVQEYVANYINADWIESLTATSERSRRLSPPAFRYQLTELARKAGKRIVLPEGDEPRTVKAAAICAERGIATCVLLGNPAEINRVAASQGVELGAGIEIVDPEVVRESYVGRLVELRKNKGMTETVAREQLEDNVVLGTLMLEQDEVDGLVSGAVHTTANTIRPPLQLIKTAPGSSLVSSVFFMLLPEQVYVYGDCAINPDPTAEQLAEIAIQSADSAAAFGIEPRVAMLSYSTGTSGAGSDVEKVREATRLAQEKRPDLMIDGPLQYDAAVMADVAKSKAPNSPVAGRATVFIFPDLNTGNTTYKAVQRSADLISIGPMLQGMRKPVNDLSRGALVDDIVYTIALTAIQSAQQQ

SEQ ID NO: 91 - protein, 345 aa, Corynebacterium glutamicum
MTTAAPQEFTAAVVEKFGHDVTVKDIDLPKPGPHQALVKVLTSGICHTDLHALEGDWPVKPEPPFVPGHEGVGEVVELGPGEHDVKVGDIVGNAWLWSACGTCEYCITGRETQCNEAEYGGYTQNGSFGQYMLVDTRYAARIPDGVDYLEAAPILCAGVTVYKALKVSETRPGQFMVISGVGGLGHIAVQYAAAMGMRVIAVDIADDKLELARKHGAEFTVNARNEDSGEAVQKYTNGGAHGVLVTAVHEAAFGQALDMARRAGTIVFNGLPPGEFPASVFNIVFKGLTIRGSLVGTRQDLAEALDFFARGLIKPTVSECSLDEVNGVLDRMRNGKIDGRVAIRF

SEQ ID NO: 92 - protein, 610 aa, Corynebacterium glutamicum
MTTAAIRGLQGEAPTKNKELLNWIADAVELFQPEAVVFVDGSQAEWDRMAEDLVEAGTLIKLNEEKRPNSYLARSNPSDVARVESRTFICSEKEEDAGPTNNWAPPQAMKDEMSKHYAGSMKGRTMYVVPFCMGPISDPDPKLGVQLTDSEYVVMSMRIMTRMGIEALDKIGANGSFVRCLHSVGAPLEPGQEDVAWPCNDTKYITQFPETKEIWSYGSGYGGNAILAKKCYALRIASVMAREEGWMAEHMLILKLINPEGKAYHIAAAFPSACGKTNLAMITPTIPGWTAQVVGDDIAWLKLREDGLYAVNPENGFFGVAPGTNYASNPIAMKTMEPGNTLFTNVALTDDGDIWWEGMDGDAPAHLIDWMGNDWTPESDENAAHPNSRYCVAIDQSPAAAPEFNDWEGVKIDAILFGGRRADTVPLVTQTYDWEHGTMVGALLASGQTAASAEAKVGTLRHDPMAMLPFIGYNAGEYLQNWIDMGNKGGDKMPSIFLVNWFRRGEDGRFLWPGFGDNSRVLKWVIDRIEGHVGADETVVGHTAKAEDLDLDGLDTPIEDVKEALTAPAEQWANDVEDNAEYLTFLGPRVPAEVHSQFDALKARISAAHA

SEQ ID NO: 93 - protein, 540 aa, Escherichia coli
MRVNNGLTPQELEAYGISDVHDIVYNPSYDLLYQEELDPSLTGYERGVLTNLGAVAVDTGIFTGRSPKDKYIVRDDTTRDTFWWADKGKGKNDNKPLSPETWQHLKGLVTRQLSGKRLFVVDAFCGANPDTRLSVRFITEVAWQAHFVKNMFIRPSDEELAGFKPDFIVMNGAKCTNPQWKEQGLNSENFVAFNLTERMQLIGGTWYGGEMKKGMFSMMNYLLPLKGIASMHCSANVGEKGDVAVFFGLSGTGKTTLSTDPKRRLIGDDEHGWDDDGVFNFEGGCYAKTIKLSKEAEPEIYNAIRRDALLENVTVREDGTIDFDDGSKTENTRVSYPIYHIDNIVKPVSKAGHATKVIFLTADAFGVLPPVSRLTADQTQYHFLSGFTAKLAGTERGITEPTPTFSACFGAAFLSLHPTQYAEVLVKRMQAAGAQAYLVNTGWNGTGKRISIKDTRAIIDAILNGSLDNAETFTLPMFNLAIPTELPGVDTKILDPRNTYASPEQWQEKAETLAKLFIDNFDKYTDTPAGAALVAAGPKL

SEQ ID NO: 94 - protein, 943 aa, Corynebacterium glutamicum
MELTVTESKNSFNAKSTLEVGDKSYDYFALSAVPGMEKLPYSLKVLGENLLRTEDGANITNEHIEAIANWDASSDPSIEIQFTPARVLMQDFTGVPCVVDLATMREAVAALGGDPNDVNPLNPAEMVIDHSVIVEAFGRPDALAKNVEIEYERNEERYQFLRWGSESFSNFRVVPPGTGIVHQVNIEYLARVVFDNEGLAYPDTCIGTDSHTTMENGLGILGWGVGGIEAEAAMLGQPVSMLIPRVVGFKLTGEIPVGVTATDVVLTITEMLRDHGVVQKFVEFYGSGVKAVPLANRATIGNMSPEFGSTCAMFPIDEETTKYLRLTGRPEEQVALVEAYAKAQGMWLDEDTVEAEYSEYLELDLSTVVPSIAGPKRPQDRILLSEAKEQFRKDLPTYTDDAVSVDTSIPATRMVNEGGGQPEGGVEADNYNASWAGSGESLATGAEGRPSKPVTVASPQGGEYTIDHGMVAIASITSCTNTSNPSVMIGAGLIARKAAEKGLKSKPWVKTICAPGSQVVDGYYQRADLWKDLEAMGFYLSGFGCTTCIGNSGPLPEEISAAINEHDLTATAVLSGNRNFEGRISPDVKMNYLASPIMVIAYAIAGTMDFDFENEALGQDQDGNDVFLKDIWPSTEEIEDTIQQAISRELYEADYADVFKGDKQWQELDVPTGDTFEWDENSTYIRKAPYFDGMPVEPVAVTDIQGARVLAKLGDSVTTDHISPASSIKPGTPAAQYLDEHGVERHDYNSLGSRRGNHEVMMRGTFANIRLQNQLVDIAGGYTRDFTQEGAPQAFIYDASVNYKAAGIPLVVLGGKEYGTGSSRDWAAKGTNLLGIRAVITESFERIHRSNLIGMGVVPLQFPAGESHESLGLDGTETFDITGLTALNEGETPKTVKVTATKENGDVVEFDAVVRIDTPGEADYYRHGGILQYVLRQMAASSK

SEQ ID NO: 95 - protein, 307 aa, Corynebacterium glutamicum
MAGLFSSAVAPTERRKALRAALAAPEIARMPGAFSPLAARAIQEAGFEGVYVSGAVVAADLALPDIGLTTLTEVAHRSRQIARVTDLPVLVDADTGFGEPMSAARTVSELEDAGVAGCHLEDQVNPKRCGHLDGKEVVGTDIMVRRIAAAVNERRDEQFVICARTDAAGVEGIDSAIERAKAYADAGADMIFTEALYSPADFEKFRAAVDIPLLANMTEFGKTELLPAQLLEDIGYNAVIYPVTLLRIAMGQVEQALGDIANTGIQTDWVDRMQHRSRLYELLRYNEYNAFDQQVFTYSADSYKPIF

SEQ ID NO: 96 - protein, 296 aa, Escherichia coli
MSLHSPGKAFRAALTKENPLQIVGTINANHALLAQRAGYQAIYLSGGGVAAGSLGLPDLGISTLDDVLTDIRRITDVCSLPLLVDADIGFGSSAFNVARTVKSMIKAGAAGLHIEDQVGAKRCGHRPNKAIVSKEEMVDRIRAAVDAKTDPDFVIMARTDALAVEGLDAAIERAQAYVEAGAEMLFPEAITELAMYRQFADAVQVPILANITEFGATPLFTTDELRSAHVAMALYPLSAFRAMNRAAEHVYNVLRQEGTQKSVIDTMQTRNELYESINYYQYEEKLDNLFARSQVK

SEQ ID NO: 97 - protein, 498 aa, Corynebacterium glutamicum
MRIHDVYTHLSADNFPKAEHLAWKFSELATDPVEVTPDVSEMIINRIIDNAAVSAASVLRRPVTVARQQAQSHPREKGGKVFGISGSYSPEWAAFANGVAVRELDFHDTFLAAEYSHPGDNIPPLLAVAQAQRSSGRDLIRGIATAYEVQVELVRGICLHEHKIDHVAHLGPSAAAGLGTLLHVDEETIYQAIGQALHTTTATRQSRKGEISSWKAFAPAFAGKMAIEAMDRAMRGEGSPAPIWEGEDGVIAWLLSGKDHVYHVPLPEHGEPKLGILETYTKEHSAEYQSQAPIDLARRMKPLVDAAGGTEHIAEIVLRTSHHTHYVIGTGANDPQKMDPQASRETLDHSIMYIFAVALQDGVWHHEFSYTRKRSTRPETVELWHKIRTVEDPEWTRRYHSDDPAKKAFGAKAVITMADGTVIEDELAVADAHPLGARPFARENYIEKFRTLAQGIVIDSEQERFLHAVQSLPDLDDLDQLNIEVDISNQAATKAGLL

SEQ ID NO: 98 - protein, 504 aa, Corynebacterium glutamicum
MINHEVRTHRSAEEFPYEEHLAHKIARVAADPVEVAADTQEMIINRIIDNASVQAASVLRRPVSSARAMAQVRPVTDGRGASVFGLPGRYAAEWAALANGTAVRELDFHDTFLAAEYSHPGDNIPPILAAAQQAGKGGKDLIRGIATGYEIQVNLVRGMCLHEHKIDHVAHLGPSAAAGIGTLLDLDVDTIYQAIGQALHTTTATRQSRKGAISSWKAFAPAFAGKMSIEAVDRAMRGEGAPSPIWEGEDGVIAWLLSGLDHIYTIPLPAEGEAKRAILDTYTKEHSAEYQSQAPIDLARSMGEKLAAQGLDLRDVDSIVLHTSHHTHYVIGTGSNDPQKFDPDASRETLDHSIMYIFAVALEDRAWHHERSYAPERAHRRETIELWNKISTVEDPEWTRRYHSVDPAEKAFGARAVITFKDGTVVEDELAVADAHPLGARPFAREQYIQKFRTLAEGVVSEKEQDRFLDAAQRTHELEDLSELNIELDADILAKAPVIPEGLF

SEQ ID NO: 99 - protein, 383 aa, Corynebacterium glutamicum
MSSATTTDVRKGLYGVIADYTAVSKVMPETNSLTYRGYAVEDLVENCSFEEVFYLLWHGELPTAQQLAEFNERGRSYRSLDAGLISLIHSLPKEAHPMDVMRTAVSYMGTKDSEYFTTDSEHIRKVGHTLLAQLPMVLAMDIRRRKGLDIIAPDSSKSVAENLLSMVFGTGPESPASNPADVRDFEKSLILYAEHSFNASTFTARVITSTKSDVYSAITGAIGALKGPLHGGANEFVMHTMLAIDDPNKAAAWINNALDNKNVVMGFGHRVYKRGDSRVPSMEKSFRELAARHDGEKWVAMYENMRDAMDARTGIKPNLDFPAGPAYHLLGFPVDFFTPLFVIARVAGWTAHIVEQYENNSLIRPLSEYNGEEQREVAPIEKR

SEQ ID NO: 100 - protein, 498 aa, Corynebacterium glutamicum; identical to SEQ ID NO: 97

SEQ ID NO: 101 - protein, 714 aa, Escherichia coli; identical to SEQ ID NO: 90

(19) United States
(12) Patent Application Publication
(10) Pub. No.: US 2020/0063172 A1
(43) Pub. Date: Feb. 27, 2020

(54) AMINO ACID PRODUCTION

(71) Applicant: Evonik Degussa GmbH, Essen (DE)

(72) Inventors: Philip Engel, Essen (DE); Steffen Schaffer, Herten (DE); Oliver Thum, Ratingen (DE); Heiko Andrea, Marl (DE); Christian Gehring, Marl (DE); Bastian Grund, Shanghai (CN)

(73) Assignee: Evonik Degussa GmbH, Essen (DE)

(21) Appl. No.: 16/546,965

(22) Filed: Aug. 21, 2019

(30) Foreign Application Priority Data
Aug. 22, 2018 (EP) 18190216.4

(51) Int. Cl. C12P 13/08; C12N 1/20

(52) U.S. Cl. CPC C12P 13/08; C12N 1/20

(57) ABSTRACT

The present invention relates to a microbial cell for producing at least one L-amino acid from at least one C1-C4 alkane, wherein the cell comprises:
(i) an increased expression relative to the wild type cell of Enzyme E₁ capable of converting the alkane to a corresponding 1-alkanol;
(ii) an increased expression relative to the wild type cell of Enzyme E₂ capable of converting the 1-alkanol of (i) to a corresponding aldehyde; and either
(iii) (A)
   an increased expression relative to the wild type cell of Enzyme E₃ capable of converting the aldehyde of (ii) to a corresponding alkanoic acid; and
   a wild-type level expression of Enzyme E₄ or an increased expression relative to the wild type cell of Enzyme E₄ capable of converting the alkanoic acid of (iii) to a corresponding fatty acyl thioester; or
(B)
   an increased expression relative to the wild type cell of Enzyme E₅ capable of converting the aldehyde of (ii) to a corresponding fatty acyl thioester;
and
(iv) an increased expression relative to the wild type cell of Enzyme E₆ capable of converting the fatty acyl thioester of (iii) to a corresponding amino acid

### FIELD OF THE INVENTION

The present invention relates to a biotechnological method for producing amino acids. In particular, the method may use alkanes as the starting material for production of L-amino acids.

### BACKGROUND OF THE INVENTION

Amino acids are especially useful as additives in animal feed and as nutritional supplements for human beings. They can also be used in infusion solutions and may function as synthetic intermediates for the manufacture of pharmaceuticals and agricultural chemicals. Compounds such as methionine, lysine, tryptophan and threonine are usually industrially produced to be used as food or feed additives and also in pharmaceuticals. In particular, methionine, an essential amino acid, which cannot be synthesized by animals, plays an important role in many body functions. L-methionine is presently being produced by chemical synthesis from hydrogen cyanide, acrolein and methyl mercaptan. These petroleum based starting materials such as acrolein and methyl mercaptan are obtained by cracking gasoline or petroleum which is bad for the environment. Also, since the costs for these starting materials will be linked to the price of petroleum, with the expected increase in petroleum prices in the future, prices of methionine will also increase relative to the increase in the petroleum prices. Similarly, lysine, an essential amino acid, also cannot be synthesized by animals. L-lysine is presently being produced by fermentation processes using high-performance strains of Corynebacterium glutamicum and Escherichia coli from sugar sources such as molasses, sucrose and/or glucose.

Production and consumption of agricultural products in general will grow particularly due to increased demand in developing countries—especially for beef and sugar. Additionally, a growing demand for bio-fuels is increasing the usage and price for sugar even further.

Since the market for amino acids will be affected by the increasing cost pressure to provide animal feed as well as the increasing price of the starting material sugar, the business will be squeezed from two sides.

There are currently four different production methods for amino acids. They include extraction, synthesis, fermentation, and enzymatic catalysis. Of these four methods, fermentation and enzymatic catalysis have the most economic and ecological advantages.

In order to maintain the competiveness of an efficient feed supplement with amino acid, there is a need to develop a production process for amino acids using an easily available and reasonably priced raw material.

Accordingly, there is a need in the art for a cheaper and more efficient biotechnological means of producing sugar-based amino acids.

### DESCRIPTION OF THE INVENTION

The present invention attempts to solve the problems above by providing a biotechnological means of producing at least one amino acid from at least one alkane. In particular, there is provided at least one genetically modified microbial cell that is capable of producing at least one amino acid from at least one alkane. The amino acid may be an L-amino acid and may be selected from the group consisting of tryptophan, lysine, threonine, methionine, O-acetyl homoserine, valine and isoleucine. The use of these genetically modified cells in a method to produce at least one amino acid may add flexibility to the production of these compounds by enabling the use of a readily available alternative petrochemical raw materials for the production of amino acids. Also, the use of whole-cell biocatalysts capable of integrating the entire means of converting alkanes to amino acids within them, makes the process of conversion simpler as only a small number of process steps are involved in the conversion. The reliance of amino acids on simple carbon sources as the carbon substrate is also eliminated.

According to one aspect of the present invention, there is provided a microbial cell for producing at least one L-amino acid from at least one short chain alkane, wherein the cell comprises:
(i) an increased expression relative to the wild type cell of Enzyme E₁ capable of converting the alkane to a corresponding 1-alkanol;
(ii) an increased expression relative to the wild type cell of Enzyme E₂ capable of converting the 1-alkanol of (i) to a corresponding aldehyde; and either
(iii) (A)
   an increased expression relative to the wild type cell of Enzyme E₃ capable of converting the aldehyde of (ii) to a corresponding alkanoic acid; and
   a wild-type level expression of Enzyme E₄ or an increased expression relative to the wild type cell of Enzyme E₄ capable of converting the alkanoic acid of (iii) to a corresponding fatty acyl thioester; or
(B)
   an increased expression relative to the wild type cell of Enzyme E₅ capable of converting the aldehyde of (ii) to a corresponding fatty acyl thioester;
and
(iv) an increased expression relative to the wild type cell of Enzyme E₆ capable of converting the fatty acyl thioester of (iii) to a corresponding amino acid

Alkanes are saturated hydrocarbons that have various applications depending on the number of carbon atoms and on the structure of the alkane (i.e. branched, linear, cyclic etc.). Alkanes (technically, always acyclic or open-chain compounds) have the general chemical formula CₙH₂ₙ₊₂. The short chain alkane used according to any aspect of the present invention may refer to at least one alkane with 1-4 carbon atoms. In particular, alkanes with 1 to 6 carbon atoms comprise, for example, methane, ethane, propane, butane, isobutene, pentane and hexane. More in particular, the short-chain alkane may be selected from the group consisting of methane, ethane, propane and butane. In one example, the short-chain alkane may be ethane, butane or propane.

Enzyme E₇

In particular, if the alkane used according to any aspect of the present invention may be a butane, the cell according to any aspect of the present invention may be genetically modified to increase expression relative to the wild type cell of at least one enzyme (E₇ₐ). More in particular, the enzyme E₇ₐ may be selected from the group consisting of acyl-ACP synthetase (E_{g}) (EC 6.2.1.20), acyl-CoA synthetase (E_{f}) (EC 6.2.1.2, EC 6.2.1.3, EC 6.2.1.10), and the combination of butyrate kinase (Eₕᵢ), (EC 2.7.2.7) and phosphotransbutyrylase (Eᵢᵢ) (EC 2.3.1.19). The increase in the expression of at least one E₇ₐ enzyme, amplifies the production of acetyl thioesters from butane. In particular, the increase in expression of at least one E₇ₐ enzyme relative to the wild-type cell intensifies the reaction: Butyrate->Butyryl-thioester->Acetyl-Thioester.

In particular, when the alkane used as a substrate according to any aspect of the present invention is a butane, the cell according to any aspect of the present invention may be genetically modified to increase the expression of at least one enzyme E₇ₐ. The enzyme E₇ₐ may be selected from the group consisting of acyl-ACP synthetase (E_{g}) (EC 6.2.1.20), acyl-CoA synthetase (E_{f}) (EC 6.2.1.2, EC 6.2.1.3, EC 6.2.1.10), and the combination of fatty acyl kinase (Eₕ) of EC 2.7.2.1, EC 2.7.2.12, EC 2.7.2.15 or EC 2.7.2.7 and phosphotransacylase (Eᵢ) of EC 2.3.1.8 or EC 2.3.1.19. In particular, enzyme E₇ₐ may be an acyl-ACP synthetase (E_{g}) comprising SEQ ID NO:21 or a variant thereof, or an acyl-CoA synthetase (E_{f}) comprising SEQ ID NO:22 or a variant thereof, or the combination of fatty acyl kinase (Eₕ) comprising SEQ ID NO:23 or a variant thereof and phosphotransacylase (Eᵢ) comprising SEQ ID NO:24 or a variant thereof.

In one example, the alkane used according to any aspect of the present invention may be a propane, the cell according to any aspect of the present invention may be genetically modified to increase expression relative to the wild type cell of at least one enzyme (E_{7b}). More in particular, the enzyme E_{7b} may be selected from the group consisting of acyl-ACP synthetase (E_{g}) (EC 6.2.1.20), acyl-CoA synthetase (E_{f}) (EC 6.2.1.2, EC 6.2.1.3, EC 6.2.1.10), methylisocitrate hydro-lyase (E_{7bi}) (EC 4.2.1.99), methylisocitrate lyase (E_{7bii}) (EC 4.1.3.30), 2-Methylisocitrate dehydratase (E_{7biii}) (EC 4.2.1.79), 2-Methylcitrate synthase (E_{7biv}) (EC 2.3.3.5), combination of phosphotranspropionylase (Eᵢᵢᵢ) (EC 2.3.1.19, EC 2.3.1.8), propionate kinase (Eₕᵢᵢ) (EC 2.7.2.15) and propionyl-CoA ligase (E_{7bvii}) (EC 6.2.1.17) and propionyl-CoA:acetate Coenzyme A transferase (E_{7bviii})(EC 2.8.3.1). The increase in the expression of at least one E_{7b} enzyme, amplifies the production of acetyl thioesters from propane. In particular, the increase in expression of at least one E_{7b} enzyme relative to the wild-type cell intensifies the reaction: Propionate->Propionyl-thioester->Acetyl-Thioester.

In particular, when the alkane used as a substrate according to any aspect of the present invention is a propane, the cell according to any aspect of the present invention may be genetically modified to increase the expression of at least one enzyme E_{7b}. The enzyme E_{7b} may be selected from the group consisting of acyl-ACP synthetase (E_{g}) (EC 6.2.1.20), acyl-CoA synthetase (E_{f}) (EC 6.2.1.2, EC 6.2.1.3, EC 6.2.1.10), methylisocitrate hydro-lyase (E_{7bi}) (EC 4.2.1.99), methylisocitrate lyase (E_{7bii}) (EC 4.1.3.30), 2-Methylisocitrate dehydratase (E_{7biii}) (EC 4.2.1.79), 2-Methylcitrate synthase (E_{7bi}) (EC 2.3.3.5), combination of phosphotranspropionylase (Eᵢᵢᵢ) (EC 2.3.1.19, EC 2.3.1.8) and propionate kinase (Eₕᵢᵢ) (EC 2.7.2.15) and propionyl-CoA ligase (E_{7bvii}) (EC 6.2.1.17). In particular, enzyme E_{7b} may be an acyl-ACP synthetase (E_{g}) comprising SEQ ID NO:21 or a variant thereof, or an acyl-CoA synthetase (E_{f}) comprising SEQ ID NO:22 or a variant thereof, methylisocitrate hydro-lyase (E_{7bi}) comprising SEQ ID NO:27, 94 or a variant thereof, a methylisocitrate lyase (E_{7bii}) comprising SEQ ID NO:28, 95, 96 or a variant thereof, a 2-Methylisocitrate dehydratase (E_{7biii}) comprising SEQ ID NO:29, 97, 98 or a variant thereof, a 2-Methylcitrate synthase (E_{7biv}) comprising SEQ ID NO:30, 99, 100 or a variant thereof or the combination of phosphotranspropionylase (Eᵢᵢᵢ) comprising SEQ ID NO:31, 101 or a variant thereof and propionate kinase (Eₕᵢᵢ) comprising SEQ ID NO:26, 4 or a variant thereof, a propionyl-CoA ligase (E_{7bvii}) (EC 6.2.1.17) comprising SEQ ID NO:32 or a variant thereof or propionyl-CoA:acetate Coenzyme A transferase (E_{7bviii}) comprising SEQ ID NO:17 a variant thereof.

The cells according to any aspect of the present invention may be used to produce amino acids from all short-chain alkanes with high space-time yield, high carbon yield and high concentration in the culture supernatant. As a result of these advantages, an efficient workup is facilitated.

The phrase “wild type” as used herein in conjunction with a cell or microorganism may denote a cell with a genome make-up that is in a form as seen naturally in the wild. The term may be applicable for both the whole cell and for individual genes. The term ‘wild type’ may thus also include cells which have been genetically modified in other aspects (i.e. with regard to one or more genes) but not in relation to the genes of interest. The term “wild type” therefore does not include such cells where the gene sequences of the specific genes of interest have been altered at least partially by man using recombinant methods. A wild type cell according to any aspect of the present invention thus refers to a cell that has no genetic mutation with respect to the whole genome and/or a particular gene. Therefore, in one example, a wild type cell with respect to enzyme E₁ may refer to a cell that has the natural/non-altered expression of the enzyme E₁ in the cell. The wild type cell with respect to enzyme E₂, E₃, E₄, E₅, E₆, E₇, etc. may be interpreted the same way and may refer to a cell that has the natural/non-altered expression of the enzyme E₂, E₃, E₄, E₅, E₆, E₇, etc. respectively in the cell. A wild-type cell can also include a cell that has mutations from nature. However, a “wild type cell” relative to a genetically modified cell according to any aspect of the present invention, means a cell in which the mutation resulting in the production of a substance in a quantifiably reduced or increased amount has not occurred. For example, a wild-type cell according to any aspect of the present invention, relative to a genetically modified cell according to any aspect of the present invention with increased expression of enzymes E₁, E₂, E₃, E₄ and E₆, E₇, refers to a cell which has not been mutated to increase the expression of enzymes E₁, E₂, E₃, E₄ and E₆, E₇, using recombinant means. Similarly, a wild-type cell according to any aspect of the present invention, relative to a genetically modified cell according to any aspect of the present invention with increased expression of enzymes E₁, E₂, E₅ and E₆, refers to a cell which has not been mutated to increase the expression of enzymes E₁, E₂, E₅ and E₆, using recombinant means. Wild-type cells are therefore, reference, or standard, cells used according to any aspect of the present invention. A wild-type cell, thus need not be a cell normally found in nature, and often will be a recombinant or genetically altered cell line. However, the wild type cells according to any aspect of the present invention may not be genetically modified with reference to the enzymes E₁, E₂, E₃, E₄, E₅, E₆, and/or E₇.

In one example, in the cell according to any aspect of the present invention, the expression of enzyme E₄ is not altered. This means, the cell used according to any aspect of the present invention, expresses E₄ in its wild type form and in the wild type form the cell expresses E₄ in a detectable amount. The wild type cell therefore, expresses enzyme E₄ and the expression is sufficient to carry out the step of converting the alkanoic acid of (iii) to a corresponding fatty acyl thioester. In this example, there is thus no need to increase the expression of E₄ and the cell expresses the wild-type E₄ in unaltered/unprocessed form.

In another example, the cell according to any aspect of the present invention may be genetically modified to increase the expression of enzyme E₄ relative to the wild type cell. The cell in this example may be genetically modified to overexpress enzyme E₄ relative to the wild-type cell so that the cell is capable of converting the alkanoic acid of (iii) to a corresponding fatty acyl thioester.

Any of the enzymes used according to any aspect of the present invention, may be an isolated enzyme. In particular, the enzymes used according to any aspect of the present invention may be used in an active state and in the presence of all cofactors, substrates, auxiliary and/or activating polypeptides or factors essential for its activity. The term “isolated”, as used herein, means that the enzyme of interest is enriched compared to the cell in which it occurs naturally. The enzyme may be enriched by SDS polyacrylamide electrophoresis and/or activity assays. For example, the enzyme of interest may constitute more than 5, 10, 20, 50, 75, 80, 85, 90, 95 or 99 percent of all the polypeptides present in the preparation as judged by visual inspection of a polyacrylamide gel following staining with Coomassie blue dye.

The enzyme used according to any aspect of the present invention may be recombinant. The term “recombinant” as used herein, refers to a molecule or is encoded by such a molecule, particularly a polypeptide or nucleic acid that, as such, does not occur naturally but is the result of genetic engineering or refers to a cell that comprises a recombinant molecule. For example, a nucleic acid molecule is recombinant if it comprises a promoter functionally linked to a sequence encoding a catalytically active polypeptide and the promoter has been engineered such that the catalytically active polypeptide is overexpressed relative to the level of the polypeptide in the corresponding wild type cell that comprises the original unaltered nucleic acid molecule.

A skilled person would be able to use any method known in the art to genetically modify a cell or microorganism. According to any aspect of the present invention, the genetically modified cell may be genetically modified so that in a defined time interval, within 2 hours, in particular within 8 hours or 24 hours, it forms at least once or twice, especially at least 10 times, at least 100 times, at least 1000 times or at least 10000 times amino acids than the wild-type cell. The increase in product formation can be determined for example by cultivating the cell according to any aspect of the present invention and the wild-type cell each separately under the same conditions (same cell density, same nutrient medium, same culture conditions) for a specified time interval in a suitable nutrient medium and then determining the amount of target product (amino acids) in the nutrient medium.

The genetically modified cell or microorganism may be genetically different from the wild type cell or microorganism. The genetic difference between the genetically modified microorganism according to any aspect of the present invention and the wild type microorganism may be in the presence of a complete gene, amino acid, nucleotide etc. in the genetically modified microorganism that may be absent in the wild type microorganism. In one example, the genetically modified microorganism according to any aspect of the present invention may comprise enzymes that enable the microorganism to produce more amino acids compared to the wild type cells. The wild type microorganism relative to the genetically modified microorganism of the present invention may have none or no detectable activity of the enzymes that enable the genetically modified microorganism to produce amino acids from alkanes. As used herein, the term ‘genetically modified microorganism’ may be used interchangeably with the term ‘genetically modified cell’. The genetic modification according to any aspect of the present invention is carried out on the cell of the microorganism.

The cells according to any aspect of the present invention are genetically transformed according to any method known in the art. In particular, the cells may be produced according to the method disclosed in WO2013024114.

The phrase ‘the genetically modified cell has an increased activity, in comparison with its wild type, in enzymes’ as used herein refers to the activity of the respective enzyme that is increased by a factor of at least 2, in particular of at least 10, more in particular of at least 100, yet more in particular of at least 1000 and even more in particular of at least 10000.

The phrase “increased activity of an enzyme”, as used herein is to be understood as increased intracellular activity. Basically, an increase in enzymatic activity can be achieved by increasing the copy number of the gene sequence or gene sequences that code for the enzyme, using a strong promoter or employing a gene or allele that codes for a corresponding enzyme with increased activity, altering the codon utilization of the gene, increasing the half-life of the mRNA or of the enzyme in various ways, modifying the regulation of the expression of the gene and optionally by combining these measures. Genetically modified cells used according to any aspect of the present invention are for example produced by transformation, transduction, conjugation or a combination of these methods with a vector that contains the desired gene, an allele of this gene or parts thereof and a vector that makes expression of the gene possible. Heterologous expression is in particular achieved by integration of the gene or of the alleles in the chromosome of the cell or an extrachromosomally replicating vector. In one example, a cell with an increased expression of an enzyme may refer to a cell with an overexpression of the enzyme relative to the wild type cell that has no or the normal expression of the enzyme. In particular, an increased activity of an enzyme relative to a wild-type cell, refers to the overexpression of the gene encoding the enzyme in the genetically modified cell.

In the same context, the phrase “decreased activity of an enzyme Eₓ” used with reference to any aspect of the present invention may be understood as meaning an activity decreased by a factor of at least 0.5, particularly of at least 0.1, more particularly of at least 0.01, even more particularly of at least 0.001 and most particularly of at least 0.0001. The phrase “decreased activity” also comprises no detectable activity (“activity of zero”). The decrease in the activity of a certain enzyme can be effected, for example, by selective mutation or by other measures known to the person skilled in the art for decreasing the activity of a certain enzyme. In particular, the person skilled in the art finds instructions for the modification and decrease of protein expression and concomitant lowering of enzyme activity by means of interrupting specific genes, for example at least in Dubeau et al. 2009. Singh & Röhm. 2008., Lee et al., 2009 and the like. The decrease in the enzymatic activity in a cell according to any aspect of the present invention may be achieved by modification of a gene comprising one of the nucleic acid sequences, wherein the modification is selected from the group comprising, consisting of, insertion of foreign DNA in the gene, deletion of at least parts of the gene, point mutations in the gene sequence, RNA interference (siRNA), antisense RNA or modification (insertion, deletion or point mutations) of regulatory sequences, such as, for example, promoters and terminators or of ribosome binding sites, which flank the gene.

Foreign DNA is to be understood in this connection as meaning any DNA sequence which is “foreign” to the gene (and not to the organism), i.e. endogenous DNA sequences can also function in this connection as “foreign DNA”. In this connection, it is particularly preferred that the gene is interrupted by insertion of a selection marker gene, thus the foreign DNA is a selection marker gene, wherein preferably the insertion was effected by homologous recombination in the gene locus.

The expression of the enzymes and genes mentioned above and all mentioned below is determinable by means of 1- and 2-dimensional protein gel separation followed by optical identification of the protein concentration in the gel with appropriate evaluation software.

If the increasing of an enzyme activity is based exclusively on increasing the expression of the corresponding gene, then the quantification of the increasing of the enzyme activity can be simply determined by a comparison of the 1- or 2-dimensional protein separations between wild type and genetically modified cell. A common method for the preparation of the protein gels with bacteria and for identification of the proteins is the procedure described by Hermann et al. (Electrophoresis, 22: 1712-23 (2001). The protein concentration can also be analysed by Western blot hybridization with an antibody specific for the protein to be determined (Sambrook et al., Molecular Cloning: a laboratory manual, 2nd Ed. Cold Spring Harbor Laboratory Press, Cold Spring Harbor, N.Y. USA, 1989) followed by optical evaluation with appropriate software for concentration determination (Lohaus and Meyer (1989) Biospektrum, 5: 32-39; Lottspeich (1999), Angewandte Chemie 111: 2630-2647). This method is also always an option when possible products of the reaction to be catalysed by the enzyme activity to be determined may be rapidly metabolized in the microorganism or else the activity in the wild type is itself too low for it to be possible adequately to determine the enzyme activity to be determined on the basis of the production formation.

In particular,
the Enzyme E₁ is selected from the group consisting of P450 alkane hydroxylase (Eₐ) of EC 1.14.15.3- and AlkB alkane hydroxylase (E_{b}) of EC 1.14.15.3;
the Enzyme E₂ is selected from the group consisting of P450 alkane hydroxylase (Eₐ) of EC 1.14.15.3-, AlkB alkane hydroxylase (E_{b}) of EC 1.14.15.3, alcohol oxidase (E_{c}) of EC 1.1.3.20 and alcohol dehydrogenase (E_{d}) of EC 1.1.1.1 or EC 1.1.1.2;
the Enzyme E₃ is selected from the group consisting of P450 alkane hydroxylase (Eₐ) of EC 1.14.15.3-, AlkB alkane hydroxylase (E_{b}) of EC 1.14.15.3, aldehyde dehydrogenase (Eₑ) of EC 1.2.1.3, EC 1.2.1.4 or EC 1.2.1.5, alcohol oxidase (E_{c}) of EC 1.1.3.20, AlkJ alcohol dehydrogenase (E_{d}) of EC 1.1.99.- and alcohol dehydrogenase (E_{d}) of EC 1.1.1.1 or EC 1.1.1.2, wherein E_{c}, E_{di} and E_{d} are each capable of oxidizing an ω-hydroxy alkanoic acid ester directly to the corresponding ω-carboxy alkanoic acid ester;
the Enzyme E₄ is selected from the group consisting of fatty acyl coenzyme A (CoA) synthase (FACS) (E_{f}) of EC 6.2.1.1, EC 6.2.1.2, EC 6.2.1.3, or EC 2.3.1.86; acyl-Acyl Carrier Protein (ACP) synthase (E_{g}) of EC 6.2.1.20 or EC 6.2.1.47; fatty acyl kinase (Eₕ) of EC 2.7.2.1, EC 2.7.2.12, EC 2.7.2.15 or EC 2.7.2.7 and phosphotransacylase (Eᵢ) of EC 2.3.1.8 or EC 2.3.1.19; and fatty acyl coenzyme A synthase (E_{f}) of EC 6.2.1.1, EC 6.2.1.2 or EC 6.2.1.3 and fatty acyl-CoA:ACP transacylase (Eⱼ) of EC 2.3.1.38 or EC 2.3.1.39;
the Enzyme E₅ is selected from the group consisting of aldehyde dehydrogenase (Eₑ) of EC 1.2.1.3, EC 1.2.1.4 or EC 1.2.1.5, alcohol oxidase (E_{c}) of EC 1.1.3.20, AlkJ alcohol dehydrogenase (E_{di}) of EC 1.1.99.- and alcohol dehydrogenase (E_{d}) of EC 1.1.1.1 or EC 1.1.1.2;
the Enzyme E₆ is capable of converting the fatty acyl thioester of any aspect of the present invention, in particular step (iii) to a corresponding amino acid.

The amino acid produced according to any aspect of the present invention may be an L-amino acid. In particular, the amino acid may be selected from the group consisting of lysine, threonine, methionine, valine, O-Acetyl homoserine, tryptophan, and isoleucine. More in particular, the amino acid produced according to any aspect of the present invention may be lysine, O-Acetyl homoserine or threonine.

Enzyme E₆

The Enzyme E₆ may be capable of converting the fatty acyl thioester of any aspect of the present invention, in particular step (iii) to a corresponding amino acid.

In one example, when the target amino acid produced according to any aspect of the present invention is lysine, the enzymes E₆ may be selected from the group consisting of aspartate kinase (E₆ₐ) (EC 2.7.2.4), aspartate semialdehyde dehydrogenase (E_{6b}) (EC 1.2.1.11), 4-hydroxy-tetrahydrodipicolinate synthase (E_{6c}) (EC 4.3.3.7), dihydrodipicolinate reductase (E_{6d}) (EC 1.17.1.8), diaminopimelate decarboxylase (E₆ₑ) (EC 4.1.1.20), lysine exporter (E_{6f}) (TCDB families 2.A.124.1.1, 2.A.75.1.1 or 2.A.75.1.2), phosphoenolpyruvate (PEP) carboxylase (E_{6g}) (EC 4.1.1.31), proton-translocating transhydrogenase (E₆ₕ) (EC 1.6.1.5), and pyruvate carboxylase (E₆ᵢ) (EC 6.4.1.1). In particular, E₆ may be an aspartate kinase (E₆ₐ) comprising SEQ ID NO:1, 79 or a variant thereof, an aspartate semialdehyde dehydrogenase (E_{6b}) comprising SEQ ID NO:2, 82 or a variant thereof, a 4-hydroxy-tetrahydrodipicolinate synthase (E_{6c}) comprising SEQ ID NO:3 or a variant thereof, a dihydrodipicolinate reductase (E_{6d}) comprising SEQ ID NO:5 or a variant thereof, a diaminopimelate decarboxylase (E₆ₑ) comprising SEQ ID NO:6 or a variant thereof, a lysine exporter (E_{6f}) comprising SEQ ID NO:7, 8, 9 or a variant thereof, phosphoenolpyruvate (PEP) carboxylase (E_{6g}) comprising SEQ ID NO:10 or a variant thereof, proton-translocating transhydrogenase (E₆ₕ) comprising SEQ ID NO:11, 20 or a variant thereof, and pyruvate carboxylase (E₆ᵢ) comprising SEQ ID NO:12 or a variant thereof. More in particular, the enzyme E₆ may be selected from the group consisting of aspartate kinase (E₆ₐ) and 4-hydroxy-tetrahydrodipicolinate synthase (E_{6c}). Even more in particular, the enzyme E₆ may comprise the sequence SEQ ID NO:1, 3 or a variant thereof. In one example, the enzyme E₆ may consists of the sequence SEQ ID NO:1, 3 or a variant thereof.

In another example, when the target amino acid produced according to any aspect of the present invention is O-Acetyl homoserine, the enzymes E₆ may be selected from the group consisting of aspartate kinase (E₆ₐ) (EC 2.7.2.4), aspartate semialdehyde dehydrogenase (E_{6b}) (EC 1.2.1.11), glyceraldehyde-3-phosphate dehydrogenase (NADP-dependent) (E₆ⱼ) (EC 1.2.1.9, EC 1.2.1.13, EC 1.2.1.59, EC 1.2.1.60), homoserine dehydrogenase (also known as a bifunctional aspartokinase I/homoserine dehydrogenase I (E₆ₖ) (EC 1.1.1.3), homoserine kinase (E₆ₗ) (EC 2.7.1.39), homoserine O-acetyltransferase (E₆ₛ) (EC 2.3.1.31), phosphoenolpyruvate (PEP) carboxylase (E_{6g}) (EC 4.1.1.31), proton-translocating transhydrogenase (E₆ₕ) (EC 1.6.1.5), pyruvate carboxylase (E₆ᵢ) (EC 6.4.1.1), 0-Acetyl homoserine exporter (E_{6ad}) (TCDB classification 2.A.42.2.2; 2.A.7.3.6; 2.A.76.1.10; 2.A.76.1.2; 2.A.79.1.1; 2.A.95.1.4, 2.A.7.21.5, 2.A.76.1.1, 2.A.76.1.9). In particular, E₆ may be an aspartate kinase (E₆ₐ) comprising SEQ ID NO:1, 79 or a variant thereof, an aspartate semialdehyde dehydrogenase (E_{6b}) comprising SEQ ID NO:2, 82 or a variant thereof, glyceraldehyde-3-phosphate dehydrogenase (NADP-dependent) (E₆ⱼ) comprising SEQ ID NO:13 or a variant thereof, homoserine dehydrogenase (E₆ₖ) comprising SEQ ID NO:14, 51, 80 or a variant thereof, homoserine kinase (E₆ₗ) comprising SEQ ID NO:15, 81 or a variant thereof, homoserine O-acetyltransferase (E₆ₛ) comprising SEQ ID NO:16, 78 or a variant thereof, phosphoenolpyruvate (PEP) carboxylase (E_{6g}) comprising SEQ ID NO:10 or a variant thereof, a proton-translocating transhydrogenase (E₆ₕ) comprising SEQ ID NO:11, 20 or a variant thereof, pyruvate carboxylase (E₆ᵢ) comprising SEQ ID NO:12 or a variant thereof, O-Acetyl homoserine exporter (E_{6ad}) comprising SEQ ID NO:19, 84, 85, 86 or variant thereof. More in particular, the enzyme E₆ may be selected from the group consisting of homoserine dehydrogenase (also known as a bifunctional aspartokinase I/homoserine dehydrogenase I (E₆ₖ) and homoserine O-acetyltransferase (E₆ₛ). Even more in particular, the enzyme E₆ may comprise the sequence SEQ ID NO:14, 51, 16, 78 or a variant thereof. In one example, the enzyme E₆ may consists of the sequence SEQ ID NO:14, 51, 16, 78 or a variant thereof.

In yet another example, when the target amino acid produced according to any aspect of the present invention is a threonine, the enzymes E₆ may be selected from the group consisting of aspartate kinase (E₆ₐ) (EC 2.7.2.4), aspartate semialdehyde dehydrogenase (E_{6b}) (EC 1.2.1.11), glyceraldehyde-3-phosphate dehydrogenase (NADP-dependent) (E₆ⱼ) (EC 1.2.1.9, EC 1.2.1.13, EC 1.2.1.59, EC 1.2.1.60), homoserine dehydrogenase (E₆ₖ) (EC 1.1.1.3), homoserine kinase (E₆ₗ) (EC 2.7.1.39), phosphoenolpyruvate (PEP) carboxylase (E_{6g}) (EC 4.1.1.31), proton-translocating transhydrogenase (E₆ₕ) (EC 1.6.1.5), pyruvate carboxylase (E₆ᵢ) (EC 6.4.1.1), threonine synthase (E₆ₘ) (EC 4.2.3.1) and threonine exporter (E₆ₙ) (TCDB families 2.A.7.3.6, 2.A.76.1.10 or 2.A.79.1.1). In particular, E₆ may be an aspartate kinase (E₆ₐ) comprising SEQ ID NO:1, 79 or variant thereof, aspartate semialdehyde dehydrogenase (E_{6b}) comprising SEQ ID NO:2, 82 or variant thereof, glyceraldehyde-3-phosphate dehydrogenase (NADP-dependent) (E₆ⱼ) comprising SEQ ID NO:13 or variant thereof, homoserine dehydrogenase (E₆ₖ) comprising SEQ ID NO:14, 51, 80 or variant thereof, homoserine kinase (E₆ₗ) comprising SEQ ID NO:15, 81 or variant thereof, phosphoenolpyruvate (PEP) carboxylase (E_{6g}) comprising SEQ ID NO:10 or variant thereof, proton-translocating transhydrogenase (E₆ₕ) comprising SEQ ID NO:11, 20 or variant thereof, pyruvate carboxylase (E₆ᵢ) comprising SEQ ID NO:12 or variant thereof, threonine synthase comprising SEQ ID NO:18, 83 or variant thereof and threonine exporter (E₆ₙ) comprising SEQ ID NO:19, 84, 85, 86 or variant thereof. More in particular, E₆ may be selected from the group consisting of a feedback-resistant variant of aspartate kinase (E₆ₐ) comprising SEQ ID NO:1 with a point mutation of T342I, or SEQ ID NO:79 with at least one point mutation selected from the group consisting of T311I, A279T, S301Y, A279V, S301F, T308I, S317A, R320G, G345D, S381F, Q404E, G408R, G277A, Q298A, T361A, E363A, and F364A, particularly with point mutation T311I, feedback-resistant variant of homoserine dehydrogenase (E₆ₖ) comprising SEQ ID NO:14 with at least one point mutation selected from the group consisting of G378E, D375A, V379E, L380E, I392P, S393A, L394P and Q399T, SEQ ID NO:51 with point mutation S345P or SEQ ID NO:80, homoserine kinase (E₆ₗ) comprising SEQ ID NO:15, 81 or a variant thereof and threonine exporter (E_{6b}) comprising SEQ ID NO:19, 84, 85, 86 or variant thereof. In one example, the enzyme E₆ may be a feedback-resistant variant of aspartate kinase (E₆ₐ), or a feedback-resistant variant of homoserine dehydrogenase (E₆ₖ). Examples of which, are provided at least in Li, Y., et al. Current status on metabolic engineering for the production of L-aspartate family amino acids and derivatives. Bioresour. Technol. (2017), particularly on page 8.

In a further example, when the target amino acid produced according to any aspect of the present invention is a methionine, the enzymes E₆ may be selected from the group consisting of aspartate kinase (E₆ₐ) (EC 2.7.2.4), aspartate semialdehyde dehydrogenase (E_{6b}) (EC 1.2.1.11), cystathionine beta-lyase (E₆ₒ) (EC 4.4.1.8), cystathionine gamma-synthase (E_{6g}) (EC 2.5.1.48), glyceraldehyde-3-phosphate dehydrogenase (NADP-dependent) (E₆ⱼ) (EC 1.2.1.9, EC 1.2.1.13, EC 1.2.1.59, EC 1.2.1.60), homocysteine transmethylase (E_{6q}) (EC 2.1.1.10 or EC 2.1.1.13), homoserine dehydrogenase (E₆ₖ) (EC 1.1.1.3), homoserine O-succinyltransferase (E₆ᵣ) (EC 2.3.1.46), homoserine O-acetyltransferase (E₆ₛ) (EC 2.3.1.31), methionine exporter (E₆ₜ) (TCDB families 2.A.3.13.1, 2.A.76.1.5 or 2.A.78.1.3), O-acetyl homoserine sulfhydrylase (E₆ᵤ) (EC 2.5.1.49), O-succinyl homoserine sulfhydrylase (E₆ᵥ) (EC:2.5.1.-), phosphoenolpyruvate (PEP) carboxylase (E_{6g}) (EC 4.1.1.31), proton-translocating transhydrogenase (E₆ₕ) (EC 1.6.1.5), and pyruvate carboxylase (E₆ᵢ) (EC 6.4.1.1). In particular, E₆ may be a feedback-resistant variant of aspartate kinase (E₆ₐ) comprising SEQ ID NO:1 with a point mutation of T342I, or SEQ ID NO:79 with at least one point mutation selected from the group consisting of T311I, A279T, S301Y, A279V, S301F, T308I, S317A, R320G, G345D, S381F, Q404E, G408R, G277A, Q298A, T361A, E363A, and F364A, particularly with point mutation T311I.

In one example, when the target amino acid produced according to any aspect of the present invention is a valine, the enzymes E₆ may be selected from the group consisting of α-acetohydroxy acid isomeroreductase (E_{6w}) (EC 1.1.1.86), acetolactate synthase (E₆ₓ) (EC 2.2.1.6) also known as a acetohydroxyacid synthase or a acetohydroxybutanoate synthase, 2,3-Dihydroxy acid hydro-lyase (E_{6y}) (EC 4.2.1.9), glucose-6-phosphate dehydrogenase (NADP-dependent) (E_{6z}) (EC 1.1.1.49, EC 1.1.1.361, EC 1.1.1.363, EC 1.1.1.388), malic enzyme (E₆ₐₐ) (EC 1.1.1.39), proton-translocating transhydrogenase (E₆ₕ) (EC 1.6.1.5), valine exporter (E_{6ab}) (TCDB classification 2.A.78.1.2, 2.A.76.1.5) and valine transaminase (E_{6ac}) EC 2.6.1.42.

In another example, when the target amino acid produced according to any aspect of the present invention is a tryptophan, the enzymes E₆ may be selected from the group consisting of anthranilate phosphoribosyl transferase (E₆ₐₑ) (EC 2.4.2.18), anthranilate synthase (E_{6af}) (EC 4.2.3.5), chorismate synthase (E_{6ag}) (EC 4.2.3.5), 2-Dehydro-3-deoxyphosphoheptonate aldolase (E₆ₐₕ) (EC 2.5.1.54), 3-Dehydroquinate synthase (E₆ₐᵢ) (EC 4.2.3.4), 3-Dehydroquinate dehydratase (E₆ₐⱼ) (EC 4.2.1.10), glucokinase (E₆ₐₖ) (EC 2.7.1.10, EC 2.7.1.1), glucose facilitator (E₆ₐₗ) (TCDB classification 2.A.1.1.1), glucose permease (E₆ₐₘ) (TCDB classification 2.A.1.1.65), indole-3-glycerol phosphate aldolase (E₆ₐₙ) (EC 4.2.1.20), indole-3-glycerol phosphate synthase (E₆ₐ₀) (EC 4.1.1.48), isocitrate lyase (E_{6ag}) (EC 4.1.3.1), malate synthase (E_{6aq}) (EC 2.3.3.9), 3-Phosphoglycerate dehydrogenase (E₆ₐᵣ) (EC 1.1.1.95, EC 1.1.1.399), phosphoribosylanthranilate isomerase (E₆ₐₛ) (EC 5.3.1.24), phosphoserine aminotransferase (E₆ₐₜ) (EC 2.6.1.52), phosphoserine phosphatase (E₆ₐᵤ) (EC 3.1.3.3), 3-Phosphoshikimate 1-carboxyvinyltransferase (E₆ₐᵥ) (EC 2.5.1.19), ribulose-5-phosphate epimerase (E_{6aw}) (EC 5.1.3.1), ribulose-5-phosphate isomerase (E₆ₐₓ) (EC 5.3.1.6), shikimate dehydrogenase (E_{6ay}) (EC 1.1.1.25, EC 1.1.1.282), shikimate kinase (E_{6az}) (EC 2.7.1.71), transaldolase (E_{6ba}) (EC 2.2.1.2), transketolase (E_{6bb}) (EC 2.2.1.1), tryptophan synthase (E_{6bc}) (EC 4.2.1.20), and tryptophan exporter (E_{6bd}) (TCDB classification 2.A.7.17.2). In particular, E₆ may selected from the group consisting of a feedback-resistant variant of anthranilate synthase (E_{6af}), a feedback-resistant variant of 2-Dehydro-3-deoxyphosphoheptonate aldolase (E₆ₐₕ), transketolase (E_{6bb}), glucose permease (E₆ₐₘ) In one example, where the enzyme E₆ is a feedback-resistant variant of anthranilate synthase (E_{6af}) or a feedback-resistant variant of 2-Dehydro-3-deoxyphosphoheptonate aldolase (E₆ₐₕ), the enzymes are disclosed at least in Li, Y., et al. Current status on metabolic engineering for the production of L-aspartate family amino acids and derivatives. Bioresour. Technol. (2017), particularly on page 8.

In a further example, when the target amino acid produced according to any aspect of the present invention is an isoleucine, the enzymes E₆ may be selected from the group consisting of aspartate kinase (E₆ₐ) (EC 2.7.2.4), aspartate semialdehyde dehydrogenase (E_{6b}) (EC 1.2.1.11), acetolactate synthase (E₆ₓ) (EC 2.2.1.6) also known as an acetohydroxyacid synthase or a acetohydroxybutanoate synthase, α-acetohydroxy acid isomeroreductase (E_{6w}) (EC 1.1.1.86), 2,3-Dihydroxy acid hydro-lyase (E_{6y}) (EC 4.2.1.19), glyceraldehyde-3-phosphate dehydrogenase (NADP-dependent) (E₆ⱼ) (EC 1.2.1.9, EC 1.2.1.13, EC 1.2.1.59, EC 1.2.1.60), homoserine dehydrogenase (E₆ₖ) (EC 1.1.1.3), homoserine kinase (E₆ₗ) (EC 2.7.1.39), isoleucine transaminase (E_{6be}) (EC 2.6.1.42), isoleucine exporter (E_{6bf}) (TCDB classification 2.A.78.1.2, 2.A.76.1.5), phosphoenolpyruvate (PEP) carboxylase (E_{6g}) (EC 4.1.1.31), pyruvate carboxylase (E₆ᵢ) (EC 6.4.1.1), PEP carboxykinase (E_{6bg}) (EC 4.1.1.32, EC 4.1.1.38, EC 4.1.1.49), threonine synthase (E₆ₘ) (EC 4.2.3.1) and threonine deaminase (E_{6bh}) (EC 4.3.1.19). In particular, E₆ may be selected from the group consisting of a feedback-resistant variant of aspartate kinase (E₆ₐ), homoserine dehydrogenase (E₆ₖ), acetolactate synthase (E₆ₓ), feedback-resistant variant of threonine dehydratase also known as threonine deaminase (E_{6bh}), homoserine kinase (E₆), α-acetohydroxy acid isomeroreductase (E_{6w}), 2,3-Dihydroxy acid hydro-lyase (E_{6y}), isoleucine transaminase (E_{6be}) and isoleucine exporter (E_{6bf}). In one example, the enzyme E₆ is a feedback-resistant variant of aspartate kinase (E₆ₐ), homoserine dehydrogenase (E₆ₖ), acetolactate synthase (E₆ₓ), or a feedback-resistant variant of threonine deaminase (E_{6bh}) also known as dehydratase, the enzymes are disclosed at least in Li, Y., et al. Current status on metabolic engineering for the production of L-aspartate family amino acids and derivatives. Bioresour. Technol. (2017), particularly on page 8.

In addition to the cells according to any aspect of the present invention being genetically modified to increase the expression of the enzymes E₄, E₂, E₃, E₄, E₅, E₆ and optionally E₇ₐ or E_{7b} depending on the substrate used, the cell according to any aspect of the present invention may also be genetically modified to decrease the expression of at least one enzyme E₈.

Enzyme E₈

In particular, the specific enzyme E₈ may be dependent on the target amino acid to be produced. Accordingly, if the cell according to any aspect of the present invention is genetically modified to produce lysine from a C1-C4 alkane, the cell is further genetically modified to decrease the expression of at least one enzyme E₈ selected from the group consisting of isocitrate dehydrogenase (E₈ⱼ) (EC 1.1.1.41, EC 1.1.1.42), lysine importer (E₈ᵣ) (TCDB classification 1.B.25.1.1, 2.A.3.1.18; 2.A.3.1.19; 2.A.3.1.2), PEP carboxykinase (E_{6bg}) (EC 4.1.1.32, EC 4.1.1.38, EC 4.1.1.49) and threonine deaminase (E_{6bh}) (EC 4.3.1.19), relative to the wild type cell. In particular, E₈ may be selected from the group consisting of isocitrate dehydrogenase (E₈ⱼ) (EC 1.1.1.41, EC 1.1.1.42), lysine importer (E₈ᵣ) (TCDB classification 1.B.25.1.1, 2.A.3.1.18; 2.A.3.1.19; 2.A.3.1.2), PEP carboxykinase (E_{6bg}) and threonine deaminase (E_{6bh}) (EC 4.3.1.19), relative to the wild type cell.

If the cell according to any aspect of the present invention is genetically modified to produce O-Acetyl homoserine from a C1-C4 alkane, the cell is further genetically modified to decrease the expression of at least one enzyme E₈ selected from the group consisting of diaminopimelate decarboxylase (E₆ₑ) (EC 4.1.1.20), homoserine kinase (E₆ₗ) (EC 2.7.1.39), homoserine O-succinyltransferase (E₆ᵣ) (EC 2.3.1.46), isocitrate dehydrogenase (E₈ⱼ) (EC 1.1.1.41, EC 1.1.1.42), PEP carboxykinase (E_{6bg}) (EC 4.1.1.32, EC 4.1.1.38, EC 4.1.1.49), threonine deaminase (E₆ₕ) (EC 4.3.1.19), O-acetyl homoserine sulfhydrylase (E₆ᵤ) (EC 2.5.1.49), O-succinyl homoserine sulfhydrylase (E₆ᵥ) (EC 2.5.1.48), and O-Acetyl homoserine importer (E₈ₖ) (TCDB classification 2.A.1.53.1, 2.A.23.4.1, 2. A.42.2.2), relative to the wild type cell.

If the cell according to any aspect of the present invention is genetically modified to produce threonine from a C1-C4 alkane, the cell is further genetically modified to decrease the expression of at least one enzyme E₈ selected from the group consisting of diaminopimelate decarboxylase (E₆ₑ) (EC 4.1.1.20), homoserine dehydrogenase (E₆ₖ) (EC 1.1.1.3), isocitrate dehydrogenase (E₆ⱼ) (EC 1.1.1.41, EC 1.1.1.42), PEP carboxykinase (E_{6bg}) (EC 4.1.1.32, EC 4.1.1.38, EC 4.1.1.49), serine hydroxymethyltransferase (E₈ₗ) (EC 2.1.2.1), threonine aldolase (E₈ₘ) (EC 4.1.2.48), threonine dehydrogenase (E₈ₙ) (EC 1.1.1.103), threonine deaminase (E_{6bh}) (EC 4.3.1.19), and threonine importer (E₈ₛ) (TCDB classification 2.A.1.53.1, 2.A.23.4.1, 2.A.42.2.2), relative to the wild type cell.

If the cell according to any aspect of the present invention is genetically modified to produce methionine from a C1-C4 alkane, the cell is further genetically modified to decrease the expression of at least one enzyme E₈ selected from the group consisting of diaminopimelate decarboxylase (E₆ₑ) (EC 4.1.1.20), homoserine kinase (E₆ₗ) (EC 2.7.1.39), isocitrate dehydrogenase (E₈ⱼ) (EC 1.1.1.41, EC 1.1.1.42), PEP carboxykinase (E_{6bg}) (EC 4.1.1.32, EC 4.1.1.38, EC 4.1.1.49), threonine deaminase (E_{6bh}) (EC 4.3.1.19), and methionine importer (E₈ₜ) (TCDB classification 2.A.22.4.3, 3.A.1.24.3; 3. A.1.24.2; 3.A.1.24.1; 3.A.1.24.4; 3.A.1.24.6; 3.A.1.3.24), relative to the wild type cell.

If the cell according to any aspect of the present invention is genetically modified to produce valine from a C1-C4 alkane, the cell is further genetically modified to decrease the expression of at least one enzyme E₈ selected from the group consisting of alanine aminotransferase (E₈ₐ) (EC 2.6.1.2, EC 2.6.1.12, EC 2.6.1.32), dihydrolipoamide acetyltransferase (E_{8b}) (EC 2.3.1.12), 2-Isopropylmalate synthase (E_{8c}) (EC 2.3.3.13), malate dehydrogenase (E_{8d}) (EC 1.1.1.37), 3-Methyl-2-oxobutanoate hydroxymethyl transferase (E₈ₑ) (EC 2.1.2.11), pantoate-beta-alanine ligase (E_{8f}) (EC 6.3.2.1), phosphoenolpyruvate (PEP) carboxylase (E_{6g}) (EC 4.1.1.31), pyruvate dehydrogenase (E_{8g}) (EC 1.2.4.1), pyruvate:quinone oxidoreductase (E₈ₕ) (EC 1.2.5.1), valine importer (E₈ᵢ) (TCDB classification 2.A.1.53.2, 2.A.26.1.9, 2.A.3.3.23, 3.A.1.4.1, 3.A.1.3.23), relative to the wild type cell.

If the cell according to any aspect of the present invention is genetically modified to produce tryptophan from a C1-C4 alkane, the cell is further genetically modified to decrease the expression of at least one enzyme E₈ selected from the group consisting of chorismate mutase (E₈ₗ) (EC 5.4.99.5), glucose-specific PEP-dependent phosphotransferase system (E₈ₘ) (EC 2.7.1.199), phosphoglucoisomerase (E₈ₙ) (EC 5.3.1.9), prephenate dehydratase (E₈ₒ) EC 4.2.1.51, pyruvate carboxylase (E₆ᵢ) (EC 6.4.1.1), pyruvate kinase (E₈ₚ) (EC 2.7.1.40) and tryptophan importer (E_{8q}) (TCDB classification 2.A.22.4.1, 2.A.22.5.3, 2.A.3.1.22, 2.A.42.1.2, 2.A.42.1.3, 2.A.88.4.1, 3.A.1.34.1, 2.A.3.1.12, 2.A.3.1.3), relative to the wild type cell.

If the cell according to any aspect of the present invention is genetically modified to produce isoleucine from a C1-C4 alkane, the cell is further genetically modified to decrease the expression of at least one enzyme E₈ selected from the group consisting of diaminopimelate decarboxylase (E₆ₑ) (EC 4.1.1.20), isocitrate dehydrogenase (E₈ⱼ) (EC 1.1.1.41, EC 1.1.1.42), isoleucine importer (E₈ᵤ) (TCDB classification 2.A.1.53.2, 2.A.26.1.9, 2.A.3.3.23, 3.A.1.4.1, 3.A.1.3.23), serine hydroxymethyltransferase (E₈ₗ) (EC 2.1.2.1), threonine aldolase (E₈ₘ) (EC 4.1.2.48), and threonine dehydrogenase (E₈ₙ) (EC 1.1.1.103), relative to the wild type cell.

Lysine

Lysine may be the target amino acid that may be produced from at least one alkane selected from the group consisting of C1-C4 alkane according to any aspect of the present invention. In particular, the cell according to any aspect of the present invention may be genetically modified to increase the expression relative to the wild type cell of at least one of the following enzymes E₁-E₆. More in particular, the cell according to any aspect of the present invention which is used to produce lysine as the target amino acid, may be genetically modified to increase the expression of all the enzymes Eᵣ E₆. Even more in particular, E₁-E₆ are:
the Enzyme E₄ is selected from the group consisting of P450 alkane hydroxylase (Eₐ) of EC 1.14.15.3- and AlkB alkane hydroxylase (E_{b}) of EC 1.14.15.3;
the Enzyme E₂ is selected from the group consisting of P450 alkane hydroxylase (Eₐ) of EC 1.14.15.3-, AlkB alkane hydroxylase (E_{b}) of EC 1.14.15.3, alcohol oxidase (E_{c}) of EC 1.1.3.20 and alcohol dehydrogenase (E_{d}) of EC 1.1.1.1 or EC 1.1.1.2;
the Enzyme E₃ is selected from the group consisting of P450 alkane hydroxylase (Eₐ) of EC 1.14.15.3-, AlkB alkane hydroxylase (E_{b}) of EC 1.14.15.3, aldehyde dehydrogenase (Eₑ) of EC 1.2.1.3, EC 1.2.1.4 or EC 1.2.1.5, alcohol oxidase (E_{c}) of EC 1.1.3.20, AlkJ alcohol dehydrogenase (E_{di}) of EC 1.1.99.- and alcohol dehydrogenase (E_{d}) of EC 1.1.1.1 or EC 1.1.1.2, wherein E_{c}, E_{di}, and E_{d} are each capable of oxidizing an co-hydroxy alkanoic acid ester directly to the corresponding co-carboxy alkanoic acid ester;
the Enzyme E₄ is selected from the group consisting of fatty acyl coenzyme A (CoA) synthase (FACS) (E_{f}) of EC 6.2.1.1, EC 6.2.1.2, EC 6.2.1.3, or EC 2.3.1.86; acyl-Acyl Carrier Protein (ACP) synthase (E_{g}) of EC 6.2.1.20 or EC 6.2.1.47; fatty acyl kinase (Eₕ) of EC 2.7.2.1, EC 2.7.2.12, EC 2.7.2.15 or EC 27.2.7 and phosphotransacylase (Eᵢ) of EC 2.3.1.8 or EC 2.3.1.19; and fatty acyl coenzyme A synthase (E_{f}) of EC 6.2.1.1, EC 6.2.1.2 or EC 6.2.1.3 and fatty acyl-CoA:ACP transacylase (Eⱼ) of EC 2.3.1.38 or EC 2.3.1.39;
the Enzyme E₅ is selected from the group consisting of aldehyde dehydrogenase (Eₑ) of EC 1.2.1.3, EC 1.2.1.4 or EC 1.2.1.5, alcohol oxidase (E_{c}) of EC 1.1.3.20, AlkJ alcohol dehydrogenase (E_{di}) of EC 1.1.99.- and alcohol dehydrogenase (E_{d}) of EC 1.1.1.1 or EC 1.1.1.2; and
the Enzyme E₆ is capable of converting the fatty acyl thioester of any aspect of the present invention, in particular step (iii) to a corresponding amino acid.

The Enzyme E₆ capable of converting the fatty acyl thioester of any aspect of the present invention, in particular step (iii) to the lysine may be selected from the group consisting of aspartate kinase (E₆ₐ) (EC 27.2.4), aspartate semialdehyde dehydrogenase (E_{6b}) (EC 1.2.1.11), 4-hydroxy-tetrahydrodipicolinate synthase (E_{6c}) (EC 4.3.37), dihydrodipicolinate reductase (E_{6d}) (EC 1.17.1.8), diaminopimelate decarboxylase (E₆ₑ) (EC 4.1.1.20), lysine exporter (E_{6f}) (TCDB families 2.A.124.1.1, 2.A.75.1.1 or 2.A.75.1.2), phosphoenolpyruvate (PEP) carboxylase (E_{6g}) (EC 4.1.1.31), proton-translocating transhydrogenase (E₆ₕ) (EC 1.6.1.5), and pyruvate carboxylase (E₆ᵢ) (EC 6.4.1.1). In particular, E₆ may be an aspartate kinase (E₆ₐ) comprising SEQ ID NO:1, 79 or a variant thereof, an aspartate semialdehyde dehydrogenase (E_{6b}) comprising SEQ ID NO:2, 82 or a variant thereof, a 4-hydroxy-tetrahydrodipicolinate synthase (E_{6c}) comprising SEQ ID NO:3 or a variant thereof, a dihydrodipicolinate reductase (E_{6d}) comprising SEQ ID NO:5 or a variant thereof, a diaminopimelate decarboxylase (E₆ₑ) comprising SEQ ID NO:6 or a variant thereof, a lysine exporter (E_{6f}) comprising SEQ ID NO:7, 8, 9 or a variant thereof, phosphoenolpyruvate (PEP) carboxylase (E_{6g}) comprising SEQ ID NO: 10 or a variant thereof, proton-translocating transhydrogenase (E₆ₕ) comprising SEQ ID NO:11, 20 or a variant thereof, and pyruvate carboxylase (E₆ᵢ) comprising SEQ ID NO:12 or a variant thereof. More in particular, the enzyme E₆ may be selected from the group consisting of aspartate kinase (E₆ₐ) and 4-hydroxy-tetrahydrodipicolinate synthase (E_{6c}). Even more in particular, the enzyme E₆ may comprise the sequence SEQ ID NO:1, 3 or a variant thereof. In one example, the enzyme E₆ may consists of the sequence SEQ ID NO:1, 3 or a variant thereof.

The cell capable of producing lysine according to any aspect of the present invention may also be genetically modified to decrease the expression of at least one enzyme E₈ selected from the group consisting of isocitrate dehydrogenase (E₈ⱼ) (EC 1.1.1.41, EC 1.1.1.42), lysine importer (E₈ᵣ) (TCDB classification 1.B.25.1.1, 2.A.3.1.18; 2.A.3.1.19; 2.A.3.1.2), PEP carboxykinase (E_{6bg}) (EC 4.1.1.32, EC 4.1.1.38, EC 4.1.1.49) and threonine deaminase (E_{6bh}) (EC 4.3.1.19), relative to the wild type cell.

Accordingly, a cell capable of producing lysine from at least one C1-C4 alkane, may be genetically modified to increase the expression of E₄, E₂, E₃, E₄, E₅, and E₆, and decrease the expression of E₈ relative to the wild type cell.

In one example, when the substrate alkane is a butane, the cell according to any aspect of the present invention used to produce lysine, may be further genetically modified to increase expression relative to the wild type cell of at least one further enzyme (E₇ₐ). More in particular, the enzyme E₇ₐ may be selected from the group consisting of acyl-ACP synthetase (E_{g}) (EC 6.2.1.20), acyl-CoA synthetase (E_{f}) (EC 6.2.1.2, EC 6.2.1.3, EC 6.2.1.10), and the combination of butyrate kinase (Eₕᵢ), (EC 227.227) and phosphotransbutyrylase (Eᵢᵢ) (EC 2.3.1.19). In particular, enzyme E₇ₐ may be an acyl-ACP synthetase (E_{g}) comprising SEQ ID NO:21 or a variant thereof, or an acyl-CoA synthetase (E_{f}) comprising SEQ ID NO:22 or a variant thereof, or the combination of butyrate kinase (Eₕᵢ) comprising SEQ ID NO:25 or a variant thereof and phosphotransacylase (Eᵢ) comprising SEQ ID NO:24 or a variant thereof.

In another example, when the substrate alkane is a propane, the cell according to any aspect of the present invention used to produce lysine, may be further genetically modified to increase expression relative to the wild type cell of at least one further enzyme (E_{7b}). More in particular, the enzyme E_{7b} may be selected from the group consisting of acyl-ACP synthetase (E_{g}) (EC 6.2.1.20), acyl-CoA synthetase (E_{f}) (EC 6.2.1.2, EC 6.2.1.3, EC 6.2.1.10), methyl isocitrate hydro-lyase (E_{7bi}) (EC 4.2.1.99), methylisocitrate lyase (E_{7bii}) (EC 4.1.3.30), 2-Methylisocitrate dehydratase (E_{7biii}) (EC 4.2.1.79), 2-Methylcitrate synthase (E_{7biv}) (EC 2.3.3.5), combination of phosphotranspropionylase (Eᵢᵢᵢ) (EC 2.3.1.19, EC 2.3.1.8) and propionate kinase (Eₕᵢᵢ) (EC 2.7.2.15) and propionyl-CoA ligase (E_{7bvii}) (EC 6.2.1.17). Even more in particular, the enzyme E_{7b} may be an acyl-ACP synthetase (E_{g}) comprising SEQ ID NO:21 or a variant thereof, or an acyl-CoA synthetase (E_{f}) comprising SEQ ID NO:22 or a variant thereof, or a methylisocitrate hydro-lyase (E_{7bi}) comprising SEQ ID NO:27, 94 or a variant thereof, a methylisocitrate lyase (E_{7bii}) comprising SEQ ID NO:28, 95, 96 or a variant thereof, a 2-Methylisocitrate dehydratase (E_{7biii}) comprising SEQ ID NO:29, 97, 98 or a variant thereof, a 2-Methylcitrate synthase (E_{7biv}) comprising SEQ ID NO:30, 99, 100 or a variant thereof or the combination of phosphotranspropionylase (Eᵢᵢᵢ) comprising SEQ ID NO:31, 101 or a variant thereof and propionate kinase (Eₕᵢᵢ) comprising SEQ ID NO:26, 4 or a variant thereof, a propionyl-CoA ligase (E_{7bvii}) (EC 6.2.1.17) comprising SEQ ID NO:32 or a variant thereof or propionyl-CoA:acetate Coenzyme A transferase (E_{7bviii}) comprising SEQ ID NO:17 a variant thereof.

In particular, according to any aspect of the present invention, the cell may be genetically modified to increase the expression of all the enzymes E₁-E₆ for production of lysine from at least one C1-C4 alkane, wherein, E₁-E₆ are:
E₄ is a butane monoxygenase (E_{c}) (EC 1.14.13.230), preferably comprising the sequences with accession numbers AAM19732.1, AAM19730.1, AAM19728.1, AAM19727.1, AAM19729.1, ABU68845.2, WP_031430811, AAM19731.1, WP_003609331.1 or variants thereof;
E₂ is an alcohol dehydrogenase (E_{d}) (EC 1.1.1.1 or EC 1.1.1.2), preferably comprising SEQ ID NO:91 or a variant thereof;
E₃ is an aldehyde dehydrogenase (Eₑ) (EC 1.2.1.3, EC 1.2.1.4 or EC 1.2.1.5), preferably comprising SEQ ID NO:42 or a variant thereof;
E₄ is fatty acyl CoA synthase (FACS) (E_{f}) (EC 6.2.1.1, EC 6.2.1.2 or EC 6.2.1.3), preferably comprising SEQ ID NO:88 or variant thereof; and
E₆ is selected from the group consisting of:
   (i) a feedback-resistant variant of aspartate kinase (E₆ₐ), preferably comprising SEQ ID NO:1 with a point mutation of T342I, or SEQ ID NO:79 with at least one point mutation selected from the group consisting of T311I, A279T, S301Y, A279V, S301F, T308I, S317A, R320G, G345D, S381F, Q404E, G408R, G277A, Q298A, T361A, E363A, and F364A, and
   (ii) a feedback-resistant variant of 4-hydroxy-tetrahydrodipicolinate synthase (E₆J (EC 4.3.3.7), preferably comprising SEQ ID NO:3 or a variant thereof comprising point mutations G84T, G250A and/or A251C;
   preferably is E₆ a combination of E₆ₐ and E_{6c}.

O-Acetyl Homoserine

O-acetyl Homoserine may be the target amino acid that may be produced from at least one alkane selected from the group consisting of C1-C4 alkane according to any aspect of the present invention. In particular, the cell according to any aspect of the present invention may be genetically modified to increase the expression relative to the wild type cell of at least one of the following enzymes E₁-E₆. More in particular, the cell according to any aspect of the present invention which is used to produce O-acetyl Homoserine as the target amino acid, may be genetically modified to increase the expression of all the enzymes E₁-E₆. Even more in particular, E₁-E₆ are:
the Enzyme E₄ is selected from the group consisting of P450 alkane hydroxylase (Eₐ) of EC 1.14.15.3- and AlkB alkane hydroxylase (E_{b}) of EC 1.14.15.3;
the Enzyme E₂ is selected from the group consisting of P450 alkane hydroxylase (Eₐ) of EC 1.14.15.3-, AlkB alkane hydroxylase (E_{b}) of EC 1.14.15.3, alcohol oxidase (E_{c}) of EC 1.1.3.20 and alcohol dehydrogenase (E_{d}) of EC 1.1.1.1 or EC 1.1.1.2;
the Enzyme E₃ is selected from the group consisting of P450 alkane hydroxylase (Eₐ) of EC 1.14.15.3-, AlkB alkane hydroxylase (E_{b}) of EC 1.14.15.3, aldehyde dehydrogenase (Eₑ) of EC 1.2.1.3, EC 1.2.1.4 or EC 1.2.1.5, alcohol oxidase (E_{c}) of EC 1.1.3.20, AlkJ alcohol dehydrogenase (E_{di}) of EC 1.1.99.- and alcohol dehydrogenase (E_{d}) of EC 1.1.1.1 or EC 1.1.1.2, wherein E_{c}, E_{di}, and E_{d} are each capable of oxidizing an co-hydroxy alkanoic acid ester directly to the corresponding co-carboxy alkanoic acid ester;
the Enzyme E₄ is selected from the group consisting of fatty acyl coenzyme A (CoA) synthase (FACS) (E_{f}) of EC 6.2.1.1, EC 6.2.1.2, EC 6.2.1.3, or EC 2.3.1.86; acyl-Acyl Carrier Protein (ACP) synthase (E_{g}) of EC 6.2.1.20 or EC 6.2.1.47; fatty acyl kinase (Eₕ) of EC 2.7.2.1, EC 2.7.2.12, EC 2.7.2.15 or EC 27.2.7 and phosphotransacylase (Eᵢ) of EC 2.3.1.8 or EC 2.3.1.19; and fatty acyl coenzyme A synthase (E_{f}) of EC 6.2.1.1, EC 6.2.1.2 or EC 6.2.1.3 and fatty acyl-CoA:ACP transacylase (Eⱼ) of EC 2.3.1.38 or EC 2.3.1.39;
the Enzyme E₅ is selected from the group consisting of aldehyde dehydrogenase (Eₑ) of EC 1.2.1.3, EC 1.2.1.4 or EC 1.2.1.5, alcohol oxidase (E_{c}) of EC 1.1.3.20, AlkJ alcohol dehydrogenase (E_{di}) of EC 1.1.99.- and alcohol dehydrogenase (E_{d}) of EC 1.1.1.1 or EC 1.1.1.2; and
the Enzyme E₆ is capable of converting the fatty acyl thioester of any aspect of the present invention, in particular step (iii) to a corresponding amino acid.

The Enzyme E₆ capable of converting the fatty acyl thioester of any aspect of the present invention, in particular step (iii) to the o-actyl homoserine may be selected from the group consisting of aspartate kinase (E₆ₐ) (EC 27.2.4), aspartate semialdehyde dehydrogenase (E_{6b}) (EC 1.2.1.11), glyceraldehyde-3-phosphate dehydrogenase (NADP-dependent) (E₆ⱼ) (EC 1.2.1.9, EC 1.2.1.13, EC 1.2.1.59, EC 1.2.1.60), homoserine dehydrogenase (E₆ₖ) (EC 1.1.1.3), homoserine kinase (E₆ₗ) (EC 2.7.1.39), phosphoenolpyruvate (PEP) carboxylase (E_{6g}) (EC 4.1.1.31), proton-translocating transhydrogenase (E₆ₕ) (EC 1.6.1.5), pyruvate carboxylase (E₆ᵢ) (EC 6.4.1.1), threonine synthase (E₆ₘ) (EC 4.2.3.1), and threonine exporter (E₆ₙ) (TCDB families 2.A.7.3.6, 2.A.76.1.10 or 2.A.79.1.1). In particular, E₆ may be an aspartate kinase (E₆ₐ) comprising SEQ ID NO:1, 79 or a variant thereof, an aspartate semialdehyde dehydrogenase (E_{6b}) comprising SEQ ID NO:2, 82 or a variant thereof, glyceraldehyde-3-phosphate dehydrogenase (NADP-dependent) (E₆ⱼ) comprising SEQ ID NO:13 or a variant thereof, homoserine dehydrogenase (E₆ₖ) comprising SEQ ID NO:14, 51, 80 or a variant thereof, homoserine kinase (E₆ₗ) comprising SEQ ID NO:15, 81 or a variant thereof, homoserine O-acetyltransferase (E₆ₛ) comprising SEQ ID NO:16, 78, 87 or a variant thereof, phosphoenolpyruvate (PEP) carboxylase (E_{6g}) comprising SEQ ID NO:10 or a variant thereof, a proton-translocating transhydrogenase (E₆ₕ) comprising SEQ ID NO:11, 20 or a variant thereof, pyruvate carboxylase (E₆ᵢ) comprising SEQ ID NO:12 or a variant thereof, O-Acetyl homoserine exporter (E_{6ad}) comprising SEQ ID NO:19, 84, 85, 86 or a variant thereof. More in particular, E₆ may be a feedback-resistant variant of aspartate kinase (E₆ₐ) comprising SEQ ID NO:1 with a point mutation of T342I, or SEQ ID NO:79 with at least one point mutation selected from the group consisting of T311I, A279T, S301Y, A279V, S301F, T308I, S317A, R320G, G345D, S381F, Q404E, G408R, G277A, Q298A, T361A, E363A, and F364A, particularly with point mutation T311I, may be a feedback-resistant variant of homoserine dehydrogenase (E₆ₖ) comprising SEQ ID NO:14 with at least one point mutation selected from the group consisting of G378E, D375A, V379E, L380E, I392P, S393A, L394P and Q399T, SEQ ID NO:51 with point mutation S345F or SEQ ID NO:80, or may be a feedback-resistant variant of homoserine O-acetyltransferase (E₆ₛ) comprising SEQ ID NO:78 with point mutation Y294C.

Even more in particular, the enzyme E₆ may be selected from the group consisting of a feedback resistant variant of homoserine dehydrogenase (also known as a bifunctional aspartokinase l/homoserine dehydrogenase I (E₆ₖ), homoserine O-acetyltransferase (E₆ₛ) and a feedback-resistant variant of aspartate kinase (E₆ₐ). Even more in particular, the enzyme E₆ may comprise the sequence SEQ ID NO:14, 51, 16, 78 or a variant thereof. In one example, the enzyme E₆ may consists of the sequence SEQ ID NO:14, 51, 16, 78 or a variant thereof.

The cell capable of producing o-acetyl homoserine according to any aspect of the present invention may also be genetically modified to decrease the expression of at least one enzyme E₈ selected from the group consisting of decarboxylase (E₆ₑ) (EC 4.1.1.20), homoserine kinase (E₆ₗ) (EC 2.7.1.39), homoserine O-succinyltransferase (E₆ᵣ) (EC 2.3.1.46), isocitrate dehydrogenase (E₈ⱼ) (EC 1.1.1.41, EC 1.1.1.42), PEP carboxykinase (E_{6bg}) (EC 4.1.1.32, EC 4.1.1.38, EC 4.1.1.49), threonine deaminase (E₆ₕ) (EC 4.3.1.19), O-acetyl homoserine sulfhydrylase (E₆ᵤ) (EC 2.5.1.49), O-succinyl homoserine sulfhydrylase (E₆ᵥ) (EC 2.5.1.48), and O-Acetyl homoserine importer (E₈ₖ) (TCDB classification 2.A.1.53.1, 2.A.23.4.1, 2.A.42.2.2), relative to the wild type cell. Accordingly, a cell capable of producing o-acetyl homoserine from at least one C1-C4 alkane, may be genetically modified to increase the expression of E₄, E₂, E₃, E₄, E₅, and E₆, and decrease the expression of E₈ relative to the wild type cell.

In one example, when the substrate alkane is a butane, the cell according to any aspect of the present invention used to produce o-acetyl homoserine, may be further genetically modified to increase expression relative to the wild type cell of at least one further enzyme (E₇ₐ). More in particular, the enzyme E₇ₐ may be selected from the group consisting of acyl-ACP synthetase (E_{g}) (EC 6.2.1.20), acyl-CoA synthetase (E_{f}) (EC 6.2.1.2, EC 6.2.1.3, EC 6.2.1.10), and the combination of butyrate kinase (Eₕᵢ), (EC 2.7.2.7) and phosphotransbutyrylase (Eᵢᵢ) (EC 2.3.1.19). In particular, enzyme E₇ₐ may be an acyl-ACP synthetase (E_{g}) comprising SEQ ID NO:21 or a variant thereof, or an acyl-CoA synthetase (E_{f}) comprising SEQ ID NO:22 or a variant thereof, or the combination of butyrate kinase (Eₕᵢ) comprising SEQ ID NO:25 or a variant thereof and phosphotransacylase (Eᵢ) comprising SEQ ID NO:24 or a variant thereof.

In another example, when the substrate alkane is a propane, the cell according to any aspect of the present invention used to produce o-acetyl homoserine, may be further genetically modified to increase expression relative to the wild type cell of at least one further enzyme (E_{7b}). More in particular, the enzyme E_{7b} may be selected from the group consisting of acyl-ACP synthetase (E_{g}) (EC 6.2.1.20), acyl-CoA synthetase (E_{f}) (EC 6.2.1.2, EC 6.2.1.3, EC 6.2.1.10), methylisocitrate hydro-lyase (E_{7bi}) (EC 4.2.1.99), methylisocitrate lyase (E_{7bii}) (EC 4.1.3.30), 2-Methylisocitrate dehydratase (E_{7biii}) (EC 4.2.1.79), 2-Methylcitrate synthase (E_{7biv}) (EC 2.3.3.5), combination of phosphotranspropionylase (Eᵢᵢᵢ) (EC 2.3.1.19, EC 2.3.1.8) and propionate kinase (Eₕᵢᵢ) (EC 2.7.2.15) and propionyl-CoA ligase (E_{7bvii}) (EC 6.2.1.17). Even more in particular, the enzyme E_{7b} may be an acyl-ACP synthetase (E_{g}) comprising SEQ ID NO:21 or a variant thereof, or an acyl-CoA synthetase (E_{f}) comprising SEQ ID NO:22 or a variant thereof, or a methylisocitrate hydro-lyase (E_{7bi}) comprising SEQ ID NO:27, 94 or a variant thereof, a methylisocitrate lyase (E_{7bii}) comprising SEQ ID NO:28, 95, 96 or a variant thereof, a 2-Methylisocitrate dehydratase (E_{7biii}) comprising SEQ ID NO:29, 97, 98 or a variant thereof, a 2-Methylcitrate synthase (E_{7biv}) comprising SEQ ID NO:30, 99, 100 or a variant thereof or the combination of phosphotranspropionylase (Eᵢᵢᵢ) comprising SEQ ID NO:31, 101 or a variant thereof and propionate kinase (Eₕᵢᵢ) comprising SEQ ID NO:26, 4 or a variant thereof, a propionyl-CoA ligase (E_{7bvii}) (EC 6.2.1.17) comprising SEQ ID NO:32 or a variant thereof or propionyl-CoA:acetate Coenzyme A transferase (E_{7bviii}) comprising SEQ ID NO:17 a variant thereof.

In particular, according to any aspect of the present invention, the cell may be genetically modified to increase the expression of all the enzymes E₁-E₆, wherein, E₁-E₆ are:
E₄ is a butane monoxygenase (E_{c}) (EC 1.14.13.230), preferably comprising the sequences with accession numbers AAM19732.1, AAM19730.1, AAM19728.1, AAM19727.1, AAM19729.1, and ABU68845.2 or variants thereof;
E₂ is an alcohol dehydrogenase (E_{d}) (EC 1.1.1.1 or EC 1.1.1.2), preferably comprising SEQ ID NO:91 or a variant thereof;
E₃ is an aldehyde dehydrogenase (Eₑ) (EC 1.2.1.3, EC 1.2.1.4 or EC 1.2.1.5), preferably comprising SEQ ID NO:42 or a variant thereof;
E₄ is fatty acyl CoA synthase (FACS) (E_{f}) (EC 6.2.1.1, EC 6.2.1.2 or EC 6.2.1.3), preferably comprising SEQ ID NO:88 or variant thereof; and
E₆ is selected from the group consisting of:
   (i) a feedback resistant variant of homoserine dehydrogenase (E₆ₖ), preferably comprising SEQ ID NO:14 with at least one point mutation selected from the group consisting of G378E, D375A, V379E, L380E, I392P, S393A, L394P and Q399T, SEQ ID NO:51 with point mutation S345P or SEQ ID NO:80,
   (ii) a feedback-resistant variant of aspartate kinase (E₆ₐ) comprising SEQ ID NO:1 with a point mutation of T342I, or SEQ ID NO:79 with at least one point mutation selected from the group consisting of T311I, A279T, S301Y, A279V, S301F, T308I, S317A, R320G, G345D, S381F, Q404E, G408R, G277A, Q298A, T361A, E363A, and F364A, and
   (iii) a feedback-resistant variant of homoserine O-acetyltransferase (E₆ₛ) comprising SEQ ID NO:78 with point mutation Y294C;
   preferably is E₆a combination of E₆ₖ, E₆ₐ and E₆ₛ.

Threonine

Threonine may be the target amino acid that may be produced from at least one alkane selected from the group consisting of C1-C4 alkane according to any aspect of the present invention. In particular, the cell according to any aspect of the present invention may be genetically modified to increase the expression relative to the wild type cell of at least one of the following enzymes E₁-E₆. More in particular, the cell according to any aspect of the present invention which is used to produce threonine as the target amino acid, may be genetically modified to increase the expression of all the enzymes E₁-E₆. Even more in particular, E₁-E₆ are:
the Enzyme E₄ is selected from the group consisting of P450 alkane hydroxylase (Eₐ) of EC 1.14.15.3- and AlkB alkane hydroxylase (E_{b}) of EC 1.14.15.3;
the Enzyme E₂ is selected from the group consisting of P450 alkane hydroxylase (Eₐ) of EC 1.14.15.3-, AlkB alkane hydroxylase (E_{b}) of EC 1.14.15.3, alcohol oxidase (E_{c}) of EC 1.1.3.20 and alcohol dehydrogenase (E_{d}) of EC 1.1.1.1 or EC 1.1.1.2;
the Enzyme E₃ is selected from the group consisting of P450 alkane hydroxylase (Eₐ) of EC 1.14.15.3-, AlkB alkane hydroxylase (E_{b}) of EC 1.14.15.3, aldehyde dehydrogenase (Eₑ) of EC 1.2.1.3, EC 1.2.1.4 or EC 1.2.1.5, alcohol oxidase (E_{c}) of EC 1.1.3.20, AlkJ alcohol dehydrogenase (E_{di}) of EC 1.1.99.- and alcohol dehydrogenase (E_{d}) of EC 1.1.1.1 or EC 1.1.1.2, wherein E_{c}, E_{di}, and E_{d} are each capable of oxidizing an co-hydroxy alkanoic acid ester directly to the corresponding co-carboxy alkanoic acid ester;
the Enzyme E₄ is selected from the group consisting of fatty acyl coenzyme A (CoA) synthase (FACS) (E_{f}) of EC 6.2.1.1, EC 6.2.1.2, EC 6.2.1.3, or EC 2.3.1.86; acyl-Acyl Carrier Protein (ACP) synthase (E_{g}) of EC 6.2.1.20 or EC 6.2.1.47; fatty acyl kinase (Eₕ) of EC 2.7.2.1, EC 2.7.2.12, EC 2.7.2.15 or EC 27.2.7 and phosphotransacylase (Eᵢ) of EC 2.3.1.8 or EC 2.3.1.19; and fatty acyl coenzyme A synthase (E_{f}) of EC 6.2.1.1, EC 6.2.1.2 or EC 6.2.1.3 and fatty acyl-CoA:ACP transacylase (Eⱼ) of EC 2.3.1.38 or EC 2.3.1.39;
the Enzyme E₅ is selected from the group consisting of aldehyde dehydrogenase (Eₑ) of EC 1.2.1.3, EC 1.2.1.4 or EC 1.2.1.5, alcohol oxidase (E_{c}) of EC 1.1.3.20, AlkJ alcohol dehydrogenase (E_{di}) of EC 1.1.99.- and alcohol dehydrogenase (E_{d}) of EC 1.1.1.1 or EC 1.1.1.2; and
the Enzyme E₆ is capable of converting the fatty acyl thioester of any aspect of the present invention, in particular step (iii) to a corresponding amino acid.

The Enzyme E₆ capable of converting the fatty acyl thioester of any aspect of the present invention, in particular step (iii) to the threonine may be selected from the group consisting of E₆ may be selected from the group consisting of aspartate kinase (E₆ₐ) (EC 27.2.4), aspartate semialdehyde dehydrogenase (E_{6b}) (EC 1.2.1.11), glyceraldehyde-3-phosphate dehydrogenase (NADP-dependent) (E₆ⱼ) (EC 1.2.1.9, EC 1.2.1.13, EC 1.2.1.59, EC 1.2.1.60), homoserine dehydrogenase (E₆ₖ) (EC 1.1.1.3), homoserine kinase (E₆ₗ) (EC 2.7.1.39), phosphoenolpyruvate (PEP) carboxylase (E_{6g}) (EC 4.1.1.31), proton-translocating transhydrogenase (E₆ₕ) (EC 1.6.1.5), pyruvate carboxylase (E₆ᵢ) (EC 6.4.1.1), threonine synthase (E₆ₘ) (EC 4.2.3.1) and threonine exporter (E₆ₙ) (TCDB families 2.A.7.3.6, 2.A.76.1.10 or 2.A.79.1.1). In particular, E₆ may be an aspartate kinase (E₆ₐ) comprising SEQ ID NO:1, 79 or variant thereof, aspartate semialdehyde dehydrogenase (E_{6b}) comprising SEQ ID NO:2, 82 or variant thereof, glyceraldehyde-3-phosphate dehydrogenase (NADP-dependent) (E₆ⱼ) comprising SEQ ID NO:13 or variant thereof, homoserine dehydrogenase (E₆ₖ) comprising SEQ ID NO:14, 51, 80 or variant thereof, homoserine kinase (E₆ₗ) comprising SEQ ID NO:15, 81 or variant thereof, phosphoenolpyruvate (PEP) carboxylase (E_{6g}) comprising SEQ ID NO: 10 or variant thereof, proton-translocating transhydrogenase (E₆ₕ) comprising SEQ ID NO:11, 20 or variant thereof, pyruvate carboxylase (E₆ᵢ) comprising SEQ ID NO:12 or variant thereof, threonine synthase comprising SEQ ID NO:18, 83 or variant thereof and threonine exporter (E₆ₙ) comprising SEQ ID NO:19, 84, 85, 86 or variant thereof. More in particular, E₆ may be selected from the group consisting of a feedback-resistant variant of aspartate kinase (E₆ₐ) comprising SEQ ID NO:1 with a point mutation of T342I, or SEQ ID NO:79 with at least one point mutation selected from the group consisting of T311I, A279T, S301Y, A279V, S301F, T308I, S317A, R320G, G345D, S381F, Q404E, G408R, G277A, Q298A, T361A, E363A, and F364A, particularly with point mutation T311I, feedback-resistant variant of homoserine dehydrogenase (E₆ₖ) comprising SEQ ID NO: 14 with at least one point mutation selected from the group consisting of G378E, D375A, V379E, L380E, I392P, S393A, L394P and Q399T, SEQ ID NO:51 with point mutation S345P or SEQ ID NO:80, homoserine kinase (E₆ₗ) comprising SEQ ID NO:15, 81 or a variant thereof and threonine exporter (E₆ₙ) comprising SEQ ID NO:19, 84, 85, 86 or variant thereof. In one example, the enzyme E₆ may be a feedback-resistant variant of aspartate kinase (E₆ₐ), or a feedback-resistant variant of homoserine dehydrogenase (E₆ₖ). Examples of which, are provided at least in Li, Y., et al. Current status on metabolic engineering for the production of L-aspartate family amino acids and derivatives. Bioresour. Technol. (2017), particularly on page 8.

The cell capable of producing threonine according to any aspect of the present invention may also be genetically modified to decrease the expression of at least one enzyme E₈ selected from the group consisting of diaminopimelate decarboxylase (E₆ₑ) (EC 4.1.1.20), homoserine dehydrogenase (E₆ₖ) (EC 1.1.1.3), isocitrate dehydrogenase (E₆ⱼ) (EC 1.1.1.41, EC 1.1.1.42), PEP carboxykinase (E_{6bg}) (EC 4.1.1.32, EC 4.1.1.38, EC 4.1.1.49), serine hydroxymethyltransferase (E₈ₗ) (EC 2.1.2.1), threonine aldolase (E₈ₘ) (EC 4.1.2.48), threonine dehydrogenase (E₈ₙ) (EC 1.1.1.103), threonine deaminase (E_{6bh}) (EC 4.3.1.19), and threonine importer (E₈ₛ) (TCDB classification 2.A.1.53.1, 2.A.23.4.1, 2.A.42.2.2), relative to the wild type cell. Accordingly, a cell capable of producing threonine from at least one C1-C4 alkane, may be genetically modified to increase the expression of E₄, E₂, E₃, E₄, E₅, and E₆, and decrease the expression of E₈ relative to the wild type cell.

In one example, when the substrate alkane is a butane, the cell according to any aspect of the present invention used to produce threonine, may be further genetically modified to increase expression relative to the wild type cell of at least one further enzyme (E₇ₐ). More in particular, the enzyme E₇ₐ may be selected from the group consisting of acyl-ACP synthetase (E_{g}) (EC 6.2.1.20), acyl-CoA synthetase (E_{f}) (EC 6.2.1.2, EC 6.2.1.3, EC 6.2.1.10), and the combination of butyrate kinase (Eₕᵢ), (EC 27.2.7) and phosphotransbutyrylase (Eᵢᵢ) (EC 2.3.1.19). In particular, enzyme E₇ₐ may be an acyl-ACP synthetase (E_{g}) comprising SEQ ID NO:21 or a variant thereof, or an acyl-CoA synthetase (E_{f}) comprising SEQ ID NO:22 or a variant thereof, or the combination of butyrate kinase (Eₕᵢ) comprising SEQ ID NO:25 or a variant thereof and phosphotransacylase (Eᵢ) comprising SEQ ID NO:24 or a variant thereof.

In another example, when the substrate alkane is a propane, the cell according to any aspect of the present invention used to produce threonine, may be further genetically modified to increase expression relative to the wild type cell of at least one further enzyme (E_{7b}). More in particular, the enzyme E_{7b} may be selected from the group consisting of acyl-ACP synthetase (E_{g}) (EC 6.2.1.20), acyl-CoA synthetase (E_{f}) (EC 6.2.1.2, EC 6.2.1.3, EC 6.2.1.10), methylisocitrate hydro-lyase (E_{7bi}) (EC 4.2.1.99), methylisocitrate lyase (E_{7bii}) (EC 4.1.3.30), 2-Methyl isocitrate dehydratase (E_{7biii}) (EC 4.2.1.79), 2-Methylcitrate synthase (E_{7biv}) (EC 2.3.3.5), combination of phosphotranspropionylase (Eᵢᵢᵢ) (EC 2.3.1.19, EC 2.3.1.8) and propionate kinase (Eₕᵢᵢ) (EC 2.7.2.15) and propionyl-CoA ligase (E_{7bvii}) (EC 6.2.1.17). Even more in particular, the enzyme E_{7b} may be an acyl-ACP synthetase (E_{g}) comprising SEQ ID NO:21 or a variant thereof, or an acyl-CoA synthetase (E_{f}) comprising SEQ ID NO:22 or a variant thereof, or a methylisocitrate hydro-lyase (E_{7bi}) comprising SEQ ID NO:27, 94 or a variant thereof, a methylisocitrate lyase (E_{7bii}) comprising SEQ ID NO:28, 95, 96 or a variant thereof, a 2-Methylisocitrate dehydratase (E_{7biii}) comprising SEQ ID NO:29, 97, 98 or a variant thereof, a 2-Methylcitrate synthase (E_{7biv}) comprising SEQ ID NO:30, 99, 100 or a variant thereof or the combination of phosphotranspropionylase (Eᵢᵢᵢ) comprising SEQ ID NO:31, 101 or a variant thereof and propionate kinase (Eₕᵢᵢ) comprising SEQ ID NO:26, 4 or a variant thereof, a propionyl-CoA ligase (E_{7bvii}) (EC 6.2.1.17) comprising SEQ ID NO:32 or a variant thereof or propionyl-CoA:acetate Coenzyme A transferase (E_{7bviii}) comprising SEQ ID NO:17 a variant thereof.

In particular, according to any aspect of the present invention, the cell may be genetically modified to increase the expression of all the enzymes E₁-E₆, wherein, E₁-E₆ are:
E₄ is a butane monoxygenase (E_{c}) (EC 1.14.13.230), preferably comprising the sequences with accession numbers AAM19732.1, AAM19730.1, AAM19728.1, AAM19727.1, AAM19729.1, and ABU68845.2 or variants thereof;
E₂ is an alcohol dehydrogenase (E_{d}) (EC 1.1.1.1 or EC 1.1.1.2), preferably comprising SEQ ID NO:91 or a variant thereof;
E₃ is an aldehyde dehydrogenase (Eₑ) (EC 1.2.1.3, EC 1.2.1.4 or EC 1.2.1.5), preferably comprising SEQ ID NO:42 or a variant thereof;
E₄ is fatty acyl CoA synthase (FACS) (E_{f}) (EC 6.2.1.1, EC 6.2.1.2 or EC 6.2.1.3), preferably comprising SEQ ID NO:88 or variant thereof; and
E₆ is selected from the group consisting of:
(i) feedback-resistant variant of homoserine dehydrogenase (E₆ₖ) comprising SEQ ID NO:14 with at least one point mutation selected from the group consisting of G378E, D375A, V379E, L380E, I392P, S393A, L394P and Q399T, or SEQ ID NO:51 with point mutation S345P;
(ii) feedback-resistant variant of aspartate kinase (E₆ₐ) comprising SEQ ID NO:1 with a point mutation of T342I, or SEQ ID NO:79 with at least one point mutation selected from the group consisting of T311I, A279T, S301Y, A279V, S301F, T308I, S317A, R320G, G345D, S381F, Q404E, G408R, G277A, Q298A, T361A, E363A, and F364A, particularly with point mutation T311I;
(iii) homoserine kinase comprising SEQ ID NO:15, 81 or a variant thereof;
(iv) threonine synthase comprising SEQ ID NO:18, 83 or variant thereof; and
(v) threonine exporter (E₆ₙ) comprising SEQ ID NO:19.

Enzyme E₄

Enzyme E₄ may be capable of converting at least one alkane to the corresponding 1-alkanol. In particular, E₄ may be at least one P450 alkane hydroxylase/monooxygenase (Eₐ) of EC 1.14.15.1, AlkB alkane hydroxylase (E_{b}) of EC 1.14.15.3, methane monooxygenase (Eₐᵢ) of EC 1.14.13.25 or EC 1.14.18.3, propane monooxygenase (Eₐᵢᵢ) of EC 1.14.13.227, and/or butane monooxygenase (Eₐᵢᵢᵢ) of EC 1.14.13.230.

The P450 alkane hydroxylase (Eₐ) is a component of a reaction system comprising
two enzyme components cytochrome P450 alkane hydroxylase and NAD(P)H cytochrome P450 oxidoreductase of EC 1.6.2.4 or
three enzyme components cytochrome P450 alkane hydroxylase of the CYP153 type, ferredoxin NAD(P)+reductases of EC 1.18.1.2 or EC 1.18.1.3 and ferredoxin.

The AlkB alkane hydroxylase (E_{1b}) is a component of a reaction system comprising
AlkB alkane hydroxylases of EC 1.14.15.3 which is a component of a reaction system comprising three enzyme components AlkB alkane hydroxylase of EC 1.14.15.3, AlkT rubredoxin NAD(P)+reductase of EC 1.18.1.1 or of EC 1.18.1.4 and rubredoxin AlkG.

The P450 alkane hydroxylase (Eₐ) may be a methane monooxygenase (Eₐᵢ) (EC 1.14.13.25 or EC 1.14.18.3), propane monooxygenase (E_{b}) (EC 1.14.13.227) or butane monooxygenase (E_{c}) (EC 1.14.13.230).

In particular, E₁ may be an AlkB alkane hydroxylase (E_{b}) also known as an alkane monooxygenase. More in particular, E₁ may comprise sequence identity of at least 50% to the alkane monooxygenase from Pseudomonas putida GPo1 encoded by alkBGT. Even more in particular, E₄ may comprise sequence identity of at least 50% to the polypeptide YP_001185946.1. More in particular, E₁ may comprise a polypeptide with sequence identity of at least 50, 60, 65, 70, 75, 80, 85, 90, 91, 94, 95, 98 or 100% to a polypeptide YP_001185946.1.

In another example, E₁ may be a butane monooxygenase (Eₐᵢᵢᵢ) of EC 1.14.13.230 that comprises a gene cluster comprising butane monooxygenase hydroxylase BMOH alpha subunit (bmoX), butane monooxygenase beta subunit (bmoY), butane monooxygenase gamma subunit (bmoZ), butane monooxygenase regulatory protein (bmoB), butane monooxygenase reductase (bmoC_1), bmoG (similar to groEL from E. coli) and three putative ORF. In particular, the butane monooxygenase (Eₐᵢᵢᵢ) may be from Thauera butanivorans. More in particular, the butane monooxygenase operon may comprise SEQ ID NO:35.

Enzyme E₂

Enzyme E₂ may be capable of converting a 1-alkanol to the corresponding 1-alkanal. In particular, E₂ may be at least one P450 alkane hydroxylases (Eₐ) of EC 1.14.15.3, AlkB alkane hydroxylases (E_{b}) of EC 1.14.15.3, alcohol oxidase (E_{c}) of EC 1.1.3.20 or alcohol dehydrogenase (E_{d}) of EC 1.1.1.1 or EC 1.1.1.2. More in particular, E₂ may be selected from the group consisting of P450 alkane hydroxylase (Eₐ), AlkB alkane hydroxylase (E_{b}), alcohol oxidase (E_{c}) of EC 1.1.3.20, AlkJ alcohol dehydrogenase (E_{di}), and alcohol dehydrogenase (E_{dii}) of EC 1.1.1.1 or EC 1.1.1.2.

In particular, E₂ may be an AlkB alkane hydroxylase (E_{b}) also known as an alkane monooxygenase. More in particular, E₂ may comprise sequence identity of at least 50% to the alkane monooxygenase from Pseudomonas putida GPo1 encoded by alkBGT. Even more in particular, E₂ may comprise sequence identity of at least 50% to the polypeptide YP_001185946.1. More in particular, E₂ may comprise a polypeptide with sequence identity of at least 50, 60, 65, 70, 75, 80, 85, 90, 91, 94, 95, 98 or 100% to a polypeptide YP_001185946.1.

In one example, E₂ may be an alcohol oxidase (E_{c}) that may be selected from the group consisting of AAS46878.1, ACX81419.1, AAS46879.1, CAB75353.1, AAS46880.1, XP_712350.1, XP_002422236.1, XP_712386.1, EEQ43775.1, CAB75351.1, CAB75352.1, XP_002548766.1, and XP_002548765.1.

In a further example, E₂ may be an AlkJ alcohol dehydrogenase (E_{di}) and may be selected from the group consisting of Q00593.1, Q9WWW2.1, ZP_00957061.1, YP_957894.1, CAC38030.1, YP_694430.1, YP_957725.1, and YP_001672216.1.

In another example, E₂ may be an alcohol dehydrogenase (E_{dii}) and may be selected from the group consisting of AdhE, AdhP, YjgB, YqhD, GldA, EutG, YiaY, AdhE, AdhP, YhhX, YahK, HdhA, HisD, SerA, Tdh, Ugd, Udg, Gmd, YefA, YbiC, YdfG, YeaU, TtuC, YeiQ, YgbJ, YgcU, YgcT, YgcV, YggP, YgjR, YliI, YqiB, YzzH, LdhA, GapA, Epd, Dld, GatD, Gcd, GlpA, GlpB, GlpC, GlpD, GpsA and YphC from bacteria, in particular E. coli.

Enzyme E₃

Enzyme E₃ may be capable of converting at least one 1-alkanal to the corresponding alkanoic acid. In particular, E₃ may be capable of converting formaldehyde, acetaldehyde, propanal and/or butanal to the corresponding fatty acid. In particular, E₃ may be selected from the group consisting of P450 alkane hydroxylases (Eₐ) of EC 1.14.15.3-, AlkB alkane hydroxylases (E_{b}) of EC 1.14.15.3, bifunctional alcohol oxidases (E_{c}) of EC 1.1.3.20, bifunctional AlkJ alcohol dehydrogenases (E_{di}) or bifunctional alcohol dehydrogenases (E_{d}) of EC 1.1.1.1 or EC 1.1.1.2, capable of oxidizing an 1-alkanol via an 1-alkanal directly to the corresponding alkanoic acid, and aldehyde dehydrogenases (Eₑ).

Enzyme E₃ may be an aldehyde dehydrogenase (Eₑ) (EC 1.2.1.3, EC 1.2.1.4 or EC 1.2.1.5), that may be capable of catalyzing the conversion of ω-oxoalkanoic acid (ester)=ω-carboxyalkanoic acid (ester).

In one example, Eₑ may be capable of specifically catalysing the following reaction: ω-oxoalkanoic acid (ester)+NAD(P)⁺=ω-carboxyalkanoic acid (ester)+NAD(P)H+H⁺

In this case, enzyme Eₑ may be an aldehyde dehydrogenase of EC 1.2.1.3, EC 1.2.1.4 or EC 1.2.1.5, and may be selected from the group consisting of Prr, Usg, MhpF, AstD, GdhA, FrmA, Feab, Asd, Sad, PuuE, GabT, YgaW, BetB, PutA, PuuC, FeaB, AldA, Prr, EutA, GabD, AldB, TynA and YneI from bacteria, in particular E. coli.

In another example, enzyme E₃ may be capable of catalysing the following reaction: ω-oxoalkanoic acid (ester)+O₂=ω-carboxyalkanoic acid (ester)+H₂O₂

In this case, E₃ may be a fatty alcohol oxidases (E_{c}) of EC 1.1.3.20.

Enzyme E₄

The Enzyme E₄ may be capable of converting at least one alkanoic acid to the corresponding fatty acyl thioester. In particular, short-chain fatty acids, such as acetic, propanoic and/or butyric acid may be converted to the corresponding fatty acyl thioester, such as fatty acyl-Coenzyme A, fatty acyl-ACP, fatty acyl-S-4-phosphopantotheine with the 4-phosphopantotheine group residing in a polypeptide chain and the like.

In particular, E₄ may be selected from the group consisting of fatty acyl coenzyme A (CoA) synthase (E_{f}) of EC 6.2.1.1, EC 6.2.1.2 or EC 6.2.1.3; Acyl-Acyl Carrier Protein (ACP) synthase (E_{g}) of EC 6.2.1.20 or EC 6.2.1.47; Fatty acyl kinase (Eₕ) of EC 2.7.2.1, EC 2.7.2.12, EC 2.7.2.15 or EC 27.2.7 and phosphotransacylase (Eⱼ) of EC 2.3.1.8 or EC 2.3.1.19; and fatty acyl coenzyme A synthase (E_{f}) of EC 6.2.1.1, EC 6.2.1.2 or EC 6.2.1.3 and fatty acyl-CoA:ACP transacylase (Eⱼ) of EC 2.3.1.38 or EC 2.3.1.39.

In particular, E₄ may be

(a) fatty acyl CoA synthase (FACS) (E_{f}) of EC 6.2.1.1, EC 6.2.1.2 or EC 6.2.1.3;

(b) acyl-acyl-ACP synthase (E_{g}) of EC 6.2.1.20 or EC 6.2.1.47;

(c) combination of fatty acyl kinase (Eₕ) of EC 2.7.2.1, EC 2.7.2.12, EC 2.7.2.15 or EC 2.7.27 and phosphotransacylase (Eᵢ) of EC 2.3.1.8 or EC 2.3.1.19; or

(d) combination of fatty acyl CoA synthase (E_{f}) of EC 6.2.1.1, EC 6.2.1.2 or EC 6.2.1.3 and fatty acyl-CoA:ACP transacylase (Eⱼ) of EC EC 2.3.1.38 or EC 2.3.1.39

The Enzyme E_{f} may be capable of catalysing the conversion of a fatty acid to acyl-CoA. A skilled person would appreciate that some fatty acyl-CoA synthase peptides will catalyse other reactions as well, for example some acyl-CoA synthase peptides will accept other substrates in addition to fatty acids. The Enzyme Eⱼ, (acyl-CoA (coenzyme A):ACP (acyl carrier protein) transacylases may be capable of catalysing the process of conversion of dodecanoyl-CoA thioester to dodecanoyl-ACP thioester.

More in particular, E₄ may be fatty acyl CoA synthase (FACS) (E_{f}) with SEQ ID NO:88 or variant thereof. In another example, E₄ may be a combination of fatty acyl kinase (Eₕ) with SEQ ID NO:89, 90 or a variant thereof and phosphotransacylase (Eᵢ) comprising SEQ ID NO:24 or a variant thereof.

Enzyme E₅

Enzyme E₅ may be capable of converting a short-chain aldehyde to a corresponding fatty acyl thioester. In particular, E₅ may convert aldehydes such as acetaldehyde, propanal or butanal to a corresponding fatty acyl thioester, such as fatty acyl-Coenzyme A, fatty acyl-ACP or fatty acyl-S-4-phosphopantotheine with the 4-phosphopantotheine group residing in a polypeptide chain and the like. Even more in particular, the Enzyme E₅ may be an aldehyde dehydrogenase (Eₑ) (EC 1.2.1.3, EC 1.2.1.4 or EC 1.2.1.5) or an alcohol oxidase (E_{c}) (EC 1.1.3.20).

The enzymes E₄ to E₈ may comprise a polypeptide sequence wherein up to 60%, preferably up to 25%, particularly up to 15%, in particular up to 10, 9, 8, 7, 6, 5, 4, 3, 2 or 1% of the amino acid residues are modified compared to the reference sequences known in the art. A skilled person may easily obtain the sequences of the relevant enzymes, E₄ to E₈ from Genebank (https://www.ncbi.nlm.nih.gov/genbank/) and using the methods known in the art obtain the cell according to any aspect of the present invention. For example, sequences labelled by accession numbers on genebank may be modified by deletion, insertion, substitution or a combination thereof and which still possess at least 50%, preferably 65%, particularly preferably 80%, in particular more than 90% of the activity of the protein with the corresponding, reference sequence, wherein 100% activity of the reference protein is understood to mean the increasing of the activity of the cells used as a biocatalyst, i.e. the quantity of substance converted per unit time based on the cell quantity used (units per gram cell dry weight [U/g CDW]) in comparison to the activity of the biocatalyst in the absence of the reference protein.

Modifications of amino acid residues of a given polypeptide sequence which lead to no significant modifications of the properties and function of the given polypeptide are known to those skilled in the art. Thus for example many amino acids can often be exchanged for one another without problems; examples of such suitable amino acid substitutions are: Ala by Ser; Arg by Lys; Asn by Gln or His; Asp by Glu; Cys by Ser; Gln by Asn; Glu by Asp; Gly by Pro; His by Asn or Gln; lie by Leu or Val; Leu by Met or Val; Lys by Arg or Gln or Glu; Met by Leu or lie; Phe by Met or Leu or Tyr; Ser by Thr; Thr by Ser; Trp by Tyr; Tyr by Trp or Phe; Val by lie or Leu. It is also known that modifications, particularly at the N- or C-terminus of a polypeptide in the form offer example amino acid insertions or deletions, often exert no significant influence on the function of the polypeptide.

The accession numbers stated in connection with the present invention mentioned throughout this specification correspond to the NCBI ProteinBank database entries with the date 27.06.2018; as a rule, the version number of the entry is identified here by “numerals” such as for example “0.1”.

All stated percentages (%) are, unless otherwise stated, mass percent.

According to any aspect of the present invention, the microbial cell may be selected from the species of bacteria, preferably selected from the group consisting of, Abiotrophia, Acaryochloris, Accumulibacter, Acetivibrio, Acetobacter, Acetohalobium, Acetonema, Achromobacter, Acidaminococcus, Acidimicrobium, Acidiphilium, Acidithiobacillus, Acidobacterium, Acidothermus, Acidovorax, Acinetobacter, Actinobacillus, Actinomyces, Actinosynnema, Aerococcus, Aeromicrobium, Aeromonas, Afipia, Aggregatibacter, Agrobacterium, Ahrensia, Akkermansia, Alcanivorax, Alicycliphilus, Alicyclobacillus, Aliivibrio, AlkaHHmriicola, Alkaliphilus, Allochromatium, Alteromonadales, Alteromonas, Aminobacterium, Aminomonas, Ammonifex, Amycolatopsis, Amycolicicoccus, Anabaena, Anaerobaculum, Anaerococcus, Anaerofustis, Anaerolinea, Anaeromyxobacter, Anaerostipes, Anaerotruncus, Anaplasma, Anoxybacillus, Aquifex, Arcanobacterium, Arcobacter, Aromatoleum, Arthrobacter, Arthrospira, Asticcacaulis, Atopobium, Aurantimonas, Azoarcus, Azorhizobium, Azospirillum, Azotobacter, Bacillus, Bartonella, Basfia, Baumannia, Bdellovibrio, Beggiatoa, Beijerinckia, Bermanella, Beutenbergia, Bifidobacterium, Bilophila, Blastopirellula, Blautia, Blochmannia, Bordetella, Borrelia, Brachybacterium, Brachyspira, Bradyrhizobium, Brevibacillus, Brevibacterium, Brevundimonas, Brucella, Buchnera, Bulleidia, Burkholderia, Butyrivibrio, Caldalkalibacillus, Caldanaerobacter, Caldicellulosiruptor, Calditerrivibrio, Caminibacter, Campylobacter, Carboxydibrachium, Carboxydothermus, Cardiobacterium, Carnobacterium, Carsonella, Catenibacterium, Catenulispora, Catonella, Caulobacter, Cellulomonas, Cellvibrio, Centipeda, Chelativorans, Chloroflexus, Chromobacterium, Chromohalobacter, Chthoniobacter, Citreicella, Citrobacter, Citromicrobium, Clavibacter, Cloacamonas, Clostridium, Collinsella, Colwellia, Comamonas, Conexibacter, Congregibacter, Coprobacillus, Coprococcus, Coprothermobacter, Coraliomargarita, Coriobacterium, corrodens, Corynebacterium, Coxiella, Crocosphaera, Cronobacter, Cryptobacterium, Cupriavidus, Cyanobium, Cyanothece, Cylindrospermopsis, Dechloromonas, Defernbacter, Dehalococcoides, Dehalogenimonas, Deinococcus, Delftia, Denitrovibrio, Dermacoccus, Desmospora, Desulfarculus, Desulphateibacillum, Desulfitobacterium, Desulfobacca, Desulfobacterium, Desulfobulbus, Desulfococcus, Desulfohalobium, Desulfomicrobium, Desulfonatronospira, Desulforudis, Desulfotalea, Desulfotomaculum, Desulfovibrio, Desulfurispirillum, Desulfurobacterium, Desulfuromonas, Dethiobacter, Dethiosulfovibrio, Dialister, Dicheiobacter, Dickeya, Dictyoglomus, Dietzia, Dinoroseobacter, Dorea, Edwardsiella, Ehrlichia, Eikenella, Elusimicrobium, Endoriftia, Enhydrobacter, Enterobacter, Enterococcus, Epulopiscium, Erwinia, Erysipelothrix, Erythrobacter, Escherichia, Ethanoligenens, Eubacterium, Eubacterium, Exiguobacterium, Faecalibacterium, Ferrimonas, Fervidobacterium, Fibrobacter, Finegoidia, Flexistipes, Francisella, Frankia, Fructobacillus, Fulvimarina, Fusobacterium, Gallibacterium, Gallionella, Gardnerella, Gemella, Gemmata, Gemmatimonas, Geobacillus, Geobacter, Geodermatophilus, Glaciecola, Gioeobacter, Glossina, Gtuconacetobacter, Gordonia, Granulibacter, Granulicatella, Grimontia, Haemophilus, Hahella, Halanaerobiumns, Haliangium, Halomonas, Halorhodospira, Halothermothrix, Halothiobacillus, Hamiltonella, Helicobacter, Heliobacterium, Herbaspirillum, Herminiimonas, Herpetosiphon, Hippea, Hirschia, Histophilus, Hodgkinia, Hoeflea, Holdemania, Hydrogenivirga, Hydrogenobaculum, Hylemonella, Hyphomicrobium, Hyphomonas, Idiomanna, Hyobacter, Intrasporangium, Isoptericola, Isosphaera, Janibacter, Janthinobacterium, Jonesia, Jonquetella, Kangiella, Ketogulonicigenium, Kineococcus, Kingella, Klebsiella, Kocuria, Konbacter, Kosmotoga, Kribbella, Ktedonobacter, Kytococcus, Labrenzia, Lactobacillus, Lactococcus, Lanbacter, Lautropia, Lawsonia, Legionella, Leifsonia, Lentisphaera, Leptolyngbya, Leptospira, Leptothrix, Leptotrichia, Leuconostoc, Liberibacter, Limnobacter, Listeria, Loktanella, Lutiella, Lyngbya, Lysinibacillus, Macrococcus, Magnetococcus, Magnetospirillum, Mahella, Mannheimia, Maricaulis, Marinithermus, Mannobacter, Marinomonas, Mariprofundus, Mantimibacter, Marvinbryantia, Megasphaera, Meiothermus, Melissococcus, Mesorhizobium, Methylacidiphilum, Methylibium, Methylobacillus, Methyiobacter, Methylobacterium, Methylococcus, Methylocystis, Methylomicrobium, Methylophaga, Methylophilales, Methylosinus, Methyloversatilis, Methylovorus, Microbacterium, Micrococcus, Microcoleus, Microcystis, Microlunatus, Micromonospora, Mitsuokella, Mobiluncus, Moorella, Moraxella, Moritella, Mycobacterium, Myxococcus, Nakamurella, Natranaerobius, Neisseria, Neorickettsia, Neptuniibacter, Nitratifractor, Nitratiruptor, Nitrobacter, Nitrococcus, Nitrosomonas, Nitrosospira, Nitrospira, Nocardia, Nocardioides, Nocardiopsis, Nodularia, Nostoc, Novosphingobium, Oceanibulbus, Oceanicaulis, Oceanicola, Oceanithermus, Oceanobacillus, Ochrobactrum, Octadecabacter, Odyssella, Oligotropha, Olsenella, Opitutus, Oribacterium, Orientia, Ornithinibacillus, Oscillatoria, Oscillochloris, Oxaiobacter, Paenibacillus, Pantoea, Paracoccus, Parascardovia, Parasutterella, Parvibaculum, Parvimonas, Parvularcula, Pasteurella, Pasteuria, Pectobacterium, Pediococcus, Pedosphaera, Pelagibaca, Peiagibacter, Peiobacter, Pelotomaculum, Peptoniphilus, Peptostreptococcus, Persephonella, Petrotoga, Phaeobacter, Phascolarctobacterium, Phenylobacterium, Photobacterium, Pirellula, Planctomyces, Planococcus, Plesiocystis, Polaromonas, Polaromonas, Polymorphum, Poiynucieobacter, Poribacteria, Prochlorococcus, Propionibacterium, Proteus, Providencia, Pseudoalteromonas, Pseudoflavonifractor, Pseudomonas, Pseudonocardia, Pseudoramibacter, Pseudovibrio, Pseudoxanthomonas, Psychrobacter, Psychromonas, Puniceispirillum, Pusillimonas, Pyramidobacter, Rahnella, Ralstonia, Raphidiopsis, Regiella, Reinekea, Renibacterium, Rhizobium, Rhodobacter, Rhodococcus, Rhodoferax, Rhodomicrobium, Rhodopirellula, Rhodopseudomonas, Rhodospirillum, Rickettsia, Rickettsiella, Riesia, Roseburia, Roseibium, Roseiflexus, Roseobacter, Roseomonas, Roseovarius, Rothia, Rubrivivax, Rubrobacter, Ruegeria, Ruminococcus, Ruthia, Saccharomonospora, Saccharophagus, Saccharopolyspora, Sagittula, Salinispora, Salmonella, Sanguibacte, Scardovia, Sebaldella, Segniliparus, Selenomonas, Serratia, Shewanella, Shigella, Shuttleworthia, Sideroxydans, Silicibacter, Simonsiella, Sinorhizobium, Slackia, Sodalis, Solibacter, Solobacterium, Sorangium, Sphaerobacter, Sphingobium, Sphingomonas, Sphingopyxis, Spirochaeta, Sporosarcina, Stackebrandtia, Staphylococcus, Starkeya, Stenotrophomonas, Stigmatella, Streptobacillus, Streptococcus, Streptomyces, Streptosporangium, Subdoligranulum, subvibrioides, Succinatimonas, Sulfitobacter, Sulfobacillus, Sulfuricurvum, Sulfurihydrogenibium, Sulfurimonas, Sulfurospirillum, Sulfurovum, Sutterella, Symbiobacterium, Synechocystis, Syntrophobacter, Syntrophobotulus, Syntrophomonas, Syntrophothermus, Syntrophus, taiwanensis, Taylorella, Teredinibacter, Terriglobus, Thalassiobium, Thauera, Thermaerobacter, Thermanaerovibrio, Thermincola, Thermoanaerobacter, Thermoanaerobacterium, Thermobaculum, Thermobifida, Thermobispora, Thermocrinis, Thermodesutphateator, Thermodesulfobacterium, Thermodesulfobium, Thermodesulfovibrio, Thermomicrobium, Thermomonospora, Thermosediminibacter, Thermosinus, Thermosipho, Thermosynechococcus, Thermotoga, Thermovibrio, Thermus, Thioalkalimicrobium, Thioalkalivibrio, Thiobacillus, Thiomicrospira, Thiomonas, Tolumonas, Treponema, tribocorum, Trichodesmium, Tropheryma, Truepera, Tsukamurella, Tuncibacter, Variovorax, Veillonella, Verminephrobacter, Verrucomicrobium, Verrucosispora, Vesicomyosocius, Vibrio, Vibrionales, Victivallis, Weissella, Wigglesworthia, Wolbachia, Wolinella, Xanthobacter, Xanthomonas, Xenorhabdus, Xylanimonas, Xylella, Yersinia, Zinderia and Zymomonas,

In particular, the microbial cell may be from E. coli. Pseudomonas sp., Pseudomonas fluorescens. Pseudomonas putida. Pseudomonas stutzeri, Acinetobacter sp., Burkholderia sp., Burkholderia thailandensis, Cyanobakterien, Klebsiella sp., Klebsiella oxytoca. Salmonella sp., Rhizobium sp. and Rhizobium meliloti. Bacillus sp., Bacillus subtilis, Clostridium sp., Corynebacterium sp., Corynebacterium glutamicum, Brevibacterium sp., Chlorella sp. and Nostoc sp. More in particular, the microbial cell may be from E. coli.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

Formation of o-Acetyl-L-Homoserine from Ethane with Escherichia coli.

For the biotransformation of ethane to o-Acetyl-L-homoserine the genetically modified strain Escherichia coli CGSC 12149 lysCfbr_Ec thrAfbr_Ec pACYC184 {PalkS} [alkS_PpGPo1]{PalkB} [bmoXYBZ_Tb PROKKA_02001_Tb PROKKA_02000_Tb bmoC_1_Tb PROKKA_01998_Tb bmoG_Tb] pBR322 {PalkS} [alkS_PpGPo1] {PalkB} [adhA_Cg aldH_Cg]{Placuv5}[metX_Cg]{Ptac}[thrA_fbr_Ec] was used. This strain harbours the following characteristics:
i. Modification of the E. coli CGSC 12149 lysC gene (SEQ ID NO:33), encoding a feedback resistant variant of aspartokinase 3.
ii. Modification of the E. coli CGSC 12149 thrA gene (SEQ ID NO:34), encoding a feedback resistant variant of bifunctional aspartokinase 1/homoserine dehydrogenase 1 (using a natural promotor).
iii. Expression of Thauera butanivorans DSM 2080 butane monooxygenase operon (SEQ ID NO:35), comprising of bmoX_Tb (butane monooxygenase hydroxylase BMOH alpha subunit), bmoY_Tb (butane monooxygenase beta subunit), bmoZ_Tb (butane monooxygenase gamma subunit), bmoB_Tb (butane monooxygenase regulatory protein), bmoC_1_Tb (butane monooxygenase reductase), bmoG_Tb (similar to groEL from E. coli) and three putative ORF PROKKA_02001_Tb, PROKKA_02000_Tb and PROKKA_01998_Tb.
iv. Expression of Corynebacterium glutamicum ATCC 13032 adhA_Cg (SEQ ID NO:36), encoding Zn-dependent alcohol dehydrogenases and aldH_Cg (SEQ ID NO:37), encoding NAD-dependent aldehyde dehydrogenases Cgl2796 genes.
v. Expression of Corynebacterium glutamicum ATCC 13032 metX gene (SEQ ID NO:38), encoding homoserine O-acetyl transferase.
vi. Modification and expression of the E. coli CGSC 12149 thrA gene (SEQ ID NO:34), encoding a feedback resistant variant of bifunctional aspartokinase 1/homoserine dehydrogenase 1 (using an overexpression system).

These characteristics were brought about by:
i. Replacement of E. coli CGSC 12149 thrA gene by another allele of thrA, encoding a feedback resistant variant of bifunctional aspartokinase 1/homoserine dehydrogenase 1 (point mutation at bp 1034 from C to T (SEQ ID NO:34), Ser345Phe SEQ ID NO:51), with pKO3 derivative 4-49 (SEQ ID NO:39).
ii. Replacement of E. coli CGSC 12149 lysC gene by another allele of lysC, encoding a feedback resistant variant aspartokinase 3 (point mutation at bp 1055 from C to T (SEQ ID NO:33), T342I (SEQ ID NO:1), with pKO3 derivative 4-47 (SEQ ID NO:40).
iii. Introduction of plasmid pACYC184 {PalkS} [alkS_PpGPo1] {PalkB} [bmoXYBZ_Tb PROKKA_02001_Tb PROKKA_02000_Tb bmoC_1_Tb PROKKA_01998_Tb bmoG_Tb](SEQ ID NO:41)
iv. Introduction of plasmid pBR322 {PalkS} [alkS_PpGPo1] {PalkB} [adhA_Cg aldH_Cg][blaA_Ec] {Placuv5}[metX_Cg]{Ptac}[thrA_fbr_Ec] (SEQ ID NO:73)

Construction of pKO3 Modification Vectors

For construction of pKO3 derivatives for gene deletion and/or allelic replacement homologous sequences up- and downstream of the target genes were amplified by PCR from genomic DNA of Escherichia coli W3110 using the following primers. Homologous ends for assembly cloning were introduced within the primers.

| | |
|---|---|
| lysCfbr homologue sequence 1 | SEQ ID NOs: 43, 44 |
| lysCfbr homologue sequence 2 | SEQ ID NOs: 45, 46 |
| thrAfbr homologue sequence 1 | SEQ ID NOs: 47, 48 |
| thrAfbr homologue sequence 2 | SEQ ID NOs: 49, 50 |

The PCR was performed with Phusion® High-Fidelity Master Mix according to the manufacturer (New England Biolabs, Ipswitch, Mass., USA). The thermal cycle profile was 3 min at 98° C. for initial denaturation, 35 cycles: 10 sec at 98° C., 30 sec at 60° C. to 68° C. (gradient), 20 sec at 72° C. and a final 10 min hold step at 72° C. Purification of PCR products was performed by gel extraction or PCR purification according to the manufacturer of purification kits (QiaQuick PCR Purification Kit and QiaQuick Gel Extraction Kit, Qiagen, Hilden, Germany). Purified PCR products were assembled into NotI restricted pKO3 plasmid using NEBuilder® HiFi DNA Assembly Master Mix according to the manufacturers manual (New England Biolabs, Ipswitch, Mass., USA). Transformation of E. coli DH10β was performed according to the manufacturer (New England Biolabs, Ipswitch, Mass., USA). The final plasmids were verified by restriction analysis and DNA sequencing.

Construction of pJAG-4-48

For construction of pCDF derivative for gene expression of thrA, encoding a feedback resistant variant of bifunctional aspartokinase 1/homoserine dehydrogenase 1 (point mutation at bp 1034 from C to T (SEQ ID NO:34), Ser345Phe, (SEQ ID NO:51) from Escherichia coli W3110 and metX_Cg, encoding Homoserine-O-Acetyltransferase from Corynebacterium glutamicum ATCC 13032 (SEQ ID NO:16) target genes were amplified by PCR from genomic DNA of Escherichia coli W3110 or Corynebacterium glutamicum ATCC 13032 (i.e. SEQ ID NO:34 or 52) respectively using the following primers. Homologous ends for assembly cloning were introduced within the primers. The point mutation of thrA that leads to a feedback resistant variant was implemented within the forward primer. The gene thrA was cloned downstream of a tac pro motor (SEQ ID NO:53) which was amplified by PCR from another vector. Following primers were used for amplification:

| | |
|---|---|
| metX_Cg | SEQ ID NOs: 54, 55 |
| tac promotor | SEQ ID NOs: 56, 57 |
| thrA part 1 | SEQ ID NOs: 58, 59 |
| thrA part 2 | SEQ ID NOs: 60, 61 |

The PCR was performed with Phusion® High-Fidelity Master Mix according to the manufacturer (New England Biolabs, Ipswitch, Mass., USA). The thermal cycle profile was 3 min at 98° C. for initial denaturation, 35 cycles: 10 sec at 98° C., 30 sec at 60° C. to 70° C. (gradient), 45 sec at 72° C. and a final 10 min hold step at 72° C. Purification of PCR products was performed by gel extraction or PCR purification according to the manufacturer of purification kits (QiaQuick PCR Purification Kit and QiaQuick Gel Extraction Kit, Qiagen, Hilden, Germany).

Purified PCR Products were Assembled into NdeI and XbaI Restricted

pJ281_alaT_C.gl._TA_C.v.(Ct) (SEQ ID NO:62) plasmid using NEBuilder® HiFi DNA Assembly Master Mix according to the manufacturers manual (New England Biolabs, Ipswitch, Mass., USA). Transformation of E. coli DH10B was performed according to the manufacturer (New England Biolabs, Ipswitch, Mass., USA). The final plasmid was verified by restriction analysis and DNA sequencing (SEQ ID NO:63).

Construction of HM-p-25

For expression of Thauera butanivorans DSM 2080 butane monooxygenase operon (SEQ ID NO:35), comprising of bmoX_Tb (butane monooxygenase hydroxylase BMOH alpha subunit), bmoY_Tb (butane monooxygenase beta subunit), bmoZ_Tb (butane monooxygenase gamma subunit), bmoB_Tb (butane monooxygenase regulatory protein), bmoC_1_Tb (butane monooxygenase reductase), bmoG_Tb (similar to groEL from E. coli) and three putative ORF PROKKA_02001_Tb, PROKKA_02000_Tb and PROKKA_01998_Tb the whole sequence was amplified from chromosomal DNA of Thauera butanivorans DSM 2018 and subcloned into a basal vector. From this vector, the whole operon was subcloned into a vector comprising a) pACYC184 backbone b) DCPK induction system (SEQ ID NO. 64) and c) full bmo operon sequence under DCPK control (SEQ ID NO:35). The final plasmid was verified by restriction analysis and DNA sequencing (SEQ ID NO:41) with sequence part b) spanning 12129 bp-36 bp, sequence part c) spanning 37-7885 bp and sequence part a) spanning the remaining vector sequence.

Construction of AH-p-125

For construction of pBR322 derivative for gene expression of Corynebacterium glutamicum ATCC 13032 adhA_Cg (SEQ ID NO:36), encoding Zn-dependent alcohol dehydrogenases and aldH_Cg (SEQ ID NO:37), encoding NAD-dependent aldehyde dehydrogenases Cgl2796 target genes were amplified by PCR from genomic DNA of C. glutamicum ATCC 13032 using the following primers. Homologous ends for assembly cloning were introduced within the primers SEQ ID NOs: 65-68.

The PCR was performed with Phusion® High-Fidelity Master Mix according to the manufacturer (New England Biolabs, Ipswitch, Mass., USA), 2 μl of 25 mM MgCl2 was added to each 25 μl reaction. The thermal cycle profile was 3 min at 98° C. for initial denaturation, 40 cycles: 10 sec at 98° C., 30 sec at 65° C.+/−1, 5° C. (gradient), 55 sec at 72° C. and a final 5 min hold step at 72° C. Purification of PCR products was performed by gel extraction or PCR purification according to the manufacturer of purification kits (QiaQuick PCR Purification Kit and QiaQuick Gel Extraction Kit, Qiagen, Hilden, Germany).

Purified PCR products were assembled into AgeI restricted AH-p-123 plasmid bringing DCPK induction system (SEQ ID NO:64) using NEBuilder® HiFi DNA Assembly Master Mix according to the manufacturers manual (New England Biolabs, Ipswitch, Mass., USA). Transformation of E. coli DH10β was performed according to the manufacturer (New England Biolabs, Ipswitch, Mass., USA). The final plasmid was verified by restriction analysis and DNA sequencing (SEQ ID NO:69).

Construction of HM-p-50

For construction of an E. coli expression vector for thrA, encoding a feedback resistant variant of aspartate kinase from E. coli W3110 and metX, encoding homoserine acetyl transferase from C. glutamicum ATCC 13032, both genes including lacUVS promotor (metX_Cg) and tac promotor (thrAfbr_Ec) were amplified by PCR from plasmid 4-52 (SEQ ID NO:70) with the primers SEQ ID NO:71 and SEQ ID NO:72.

Purified PCR products were assembled into Sail restricted AH-p-125 (SEQ ID NO:69) plasmid using NEBuilder® HiFi DNA Assembly Master Mix according to the manufacturers manual (New England Biolabs, Ipswitch, Mass., USA). Transformation of E. coli DH10β was performed according to the manufacturer (New England Biolabs, Ipswitch, Mass., USA). The final plasmid was verified by restriction analysis and DNA sequencing (SEQ ID NO:73).

Construction of Strain GAO-EC-147

E. coli CGSC 12149 wild type was modified according to pKO3 procedure (Link A J, Phillips D, Church G M. J Bateriol. 179(20):6228-37) with plasmids according to SEQ ID NO:39 and SEQ ID NO:40. Two rounds of modifications lead to E. coli CGSC 12149 lysCfbr_EcthrAfbr_Ec. This strain was transformed with plasmids according to SEQ ID NOs: 74 and 75. Transformation of E. coli derivatives was performed via electroporation as known in the art. This work resulted in E. coli strain GAO-EC-147.

DASGIP Testing GAO-EC-147

Materials and Methods

Working with highly combustible gases in atmospheres containing significant amounts of oxygen (air for example) requires some special safety precautions. Generally, gassing of the fermenters is done with an ethane/air mixtures above the upper explosion limit (UEL) of ≈15 vol. % ethane in air. The composition of the gas mix is ethane/air 0.25/0.75.

All biotransformation experiments were conducted in a DASGIP-fermenter system in glass vessels with a working volume of 150-300 ml. Two 8 fold pump modules are connected to the fermenters. Those can either be used for a two side pH-control of eight fermenters in parallel or for a pH control with base plus glucose feeding. A third external pump can be used additionally with a constant feeding rate; this pump is not connected and controlled by the DASGIP control programme.

All vessels are equipped with a pH and a dO2 probe. Those probes are connected to a control module and the corresponding signals serve as trigger for acid/base feed for pH control and for the stirrers for dO2 control respectively. In order to avoid possible sources of ignition that could occur with conventionally used thermos blocs, the temperature is controlled by immersion of the vessels into a tempered water bath. For the same reason—elimination of ignition sources—no overhead stirrers, but submergible magnetic stirrers are used for agitation of the fermenter content.

Media

(i) LB-Medium:

25 g LB-broth are dissolved in distilled water and autoclaved for 20 min. at 121° C.

(ii) M9-Medium without C-Source:

For 1 L medium, 8.52 g Na2HPO4, 3.00 g KH2PO4, 0.50 g NaCl, and 2.00 g NH4Cl are dissolved in approximately 900 mL distilled water. pH is adjusted to 7.0 with a diluted NH3-solution and distilled water is added to a final volume of 1000 ml. The solution is autoclaved and 2 ml of a MgSO4 solution (1 mol/L) and 1 ml of US3 trace element solution are added under sterile conditions.

(iii) Trace Element Solution US3:

For 1000 ml trace element solution US3, 40 ml HCl (37%), 1.9 g MnCl2*4 H2O, 1.9 g ZnSO4*7 H2O, 0.9 g Na-EDTA*2 H2O, 0.3 g H3BO3, 0.3 g Na2MoO4*2 H2O, 4.7 g CaCl2*2 H2O, 17.8 g FeSO4*7H2O, 0.2 g CuCl2*2H2O are dissolved one by one in 900 ml distilled water. Distilled water is added to a final volume of 1000 ml and the solution is filter sterilised (0.22 μm, PTFE membrane).

(iv) MgSO4-Solution (1M):

246.47 g MgSO4*7H2O were dissolved in 1 L distilled water and filter sterilised (0.22 μm, PTFE membrane).

(v) MgSO4-Solution (200 g/L):

200 g MgSO4*7H2O were dissolved in 1 L distilled water and filter sterilised (0.22 μm, PTFE membrane).

(vi) NH4Cl-Solution (220 g/L):

220 g NH4Cl were dissolved in 1 L distilled water and filter sterilised (0.22 μm, PTFE membrane).

(vii) Glucose Feed:

550 g glucose*H2O were dissolved at ═° C. in distilled water to give a final volume of 850 ml. The solution was sterilised by autoclaving it at 121° C. for 20 min. For a glucose feed solution, 150 ml of sterile, distilled water were added under sterile conditions.

Growth and Induction in Fermenter

For experiments with growth and induction in the main DASGIP-fermenter, only one preculture step is required. 100 ml shaking flasks are filled with 25 ml LB-medium, the respective amount of antibiotic and inoculated from a cryo culture. After cultivation at 37° C. and 180 rpm, fermenters are inoculated from the LB-preculture with an OD of 0.1. The fermenters contain 190 ml M9 medium with a batch glucose concentration of 4 g/L and an antibiotic according to the cultivated strain. When the measured dO₂-increases due to glucose depletion, the glucose feed is started (0.4 g/Lh) and the inductor is added to the fermenter (1.5 μl DCPK, 1 mM IPTG, approximately after 22 h). Gas flow was set to 4.5 NL/H, after 25 h glucose feed was shut down and cultures were growing on ethane as sole carbon source. DO was set at 30% as lower level and controlled by stirring speed, pH was set up 7.0 and controlled by 220 g/L NH₄Cl when necessary.

Analytics

Quantification of Ethanol and Acetate by HPLC

The quantification of ethanol and acetate in fermentation samples is carried out by HPLC. The quantification is based on an external calibration with the respective standards.

Chemicals

Ethanol (e.g. Sigma-Aldrich, >99% (GC), purum); natrium acetate (e.g. Merck); sulfuric acid (e.g. Merck); deionized water (Purification by a Millipore system)

Sample Preparation

The aqueous fermentation samples are sterile-filtered and diluted by 20 mmolar aqueous sulfuric acid. Possible precipitates are separated by centrifugation.

HPLC Conditions

| | |
|---|---|
| HPLC system | Agilent Technologies 1200 Series |
| HPLC column | Aminex HPX-87H (300 mm × 7.8 mm) (Bio-rad) |
| Eluent | 10 mmolar aqueous sulfuric acid |
| Column temperature | 40° C. |
| Flow rate | 0.6 mL/min |
| Detector | RID (Agilent G1362A-B) and DAD (210 nm) |
| | (Agilent G1315C-B) |
| Detector temperature | 35° C. (RID) |
| Injection volume | 20 μL |
| Retention times | acetate 14.5 min; ethanol 20.5 min |

Quantification of Amino Acids by HPLC

The quantification of amino acids is carried out by HPLC after derivatization with ortho-phthaldialdehyde. The quantification is based on an external calibration with the respective standards.

Chemicals

NaOH 32% (e.g., Fluka); methanol HPLC grade (e.g. Honeywell); n-propanol (e.g. Sigma-Aldrich); o-phthaldialdehyde (e.g. Roth); boric acid (e.g. Merck); mercaptoethanol (e.g. Sigma-Aldrich); formic acid (e.g. Sigma-Aldrich); acetonitrile HPLC grade (e.g. Sigma-Aldrich); Brij35 25% in water (e.g. Sigma-Aldrich); deionized water (Purification by a Millipore system); aspartic acid (e.g. Sigma-Aldrich); homoserine (e.g. Sigma-Aldrich); threonine (e.g. Sigma-Aldrich); glycine (e.g. Merck); acetylhomoserine (e.g. Chemos); methionine (e.g. Acros); valine (e.g. Merck; isoleucine (e.g. Roth); lysine (e.g. Sigma-Aldrich);

Preparation of OP a Reagent

1000 mg o-phthaldialdehyde is dissolved in 10 ml methanol, 90 ml borate buffer (pH 10.4) is added, 500 μl mercaptoethanol is added. The reagent is stored in the fridge overnight. Then 100 μl mercaptoethanol is added.

Preparation of Borat Buffer (0.4 Mol/L)

38.1 g Na₂B₄O₇*10 H₂O is dissolved in 1 L water, pH value is adjusted to 10.4 by 10 mol/L NaOH, 1 mL Brij35 25% is added

Sample Preparation

The fermentation samples are diluted by n-propanol and centrifuged. The clear supernatant is used for analysis.

HPLC conditions

| | |
|---|---|
| HPLC system | Agilent Technologies 1200 Series |
| pre column | HPLC KrudKatcher Ultra HPLC In-Line Filter; |
| | 0.5 u Porosity × 0.004 ID (Phenomenex) |
| column | Kinetex XB-C18; 100 × 4.6 mm; 2.6 μm; |
| | 100A; (Phenomenex) |
| Eluent A | 95% water, 5% methanol, 0.1% formic acid |
| Eluent B | 90% acetonitrile, 5% water, 5% methanol, |
| | 0.1% formic acid |

Gradient Profile

| | time | eluent B | flow rate | max. pressure |
|---|---|---|---|---|
| | [min] | [%] | [ml/min] | [bar] |
| 1 | 0 | 10 | 0.6 | 400 |
| 2 | 1 | 10 | 0.6 | 400 |
| 3 | 5.5 | 35 | 0.6 | 400 |
| 4 | 6.5 | 35 | 0.6 | 400 |
| 5 | 13 | 70 | 0.6 | 400 |
| 6 | 13.1 | 100 | 0.6 | 400 |
| 7 | 16 | 100 | 0.6 | 400 |
| 8 | 16.1 | 10 | 0.6 | 400 |
| 9 | 21 | 10 | 0.6 | 400 |

| Column | 30° C. | |
|---|---|---|
| temperature | | |
| Flow rate | | 0.6 mL/min |
| Detector | | FLD (Agilent G1321A) |
| | | PMT Gain 5, excitation wavelength |
| | | 330 mm, emission wavelength 450 nm |
| Injection | # | Command |
| program | 1 | DRAW 4.5 μL from Vial 1*, def. speed, |
| (derivati- | | def. offset |
| zation) | 2 | DRAW 1.5 μL from sample, def. speed, |
| | | def. offset |
| | 3 | DRAW 0.5 μL from air, def. speed |
| | 4 | NEEDLE wash in flush Port. 15.0 sec |
| | 5 | DRAW 4.5 μL from Vial 1, def. speed, |
| | | def. offset |
| | 6 | MIX 11.0 μL in seat, def. speed, 1 times |
| | 7 | WAIT 1.00 min |
| | 8 | INJECT |
| | 9 | WAIT 0.50 min |
| | 10 | Switch VALVE to “Bypass” |
| | 11 | NEEDLE wash in flush Port. 10.0 sec |
| | 12 | Draw 100.0 μL from Vial 2*, def. speed, |
| | | def. offset |
| | 13 | EjeCt 100.0 μL from Vial 2, def. speed, |
| | | def. offset |
| | 14 | Draw 100.0 μL from Vial 3*, def. speed, |
| | | def. offset |
| | 15 | EjeCt 100.0 μL from Vial 3, def. speed, |
| | | def. offset |
| | 16 | Valve mainpass |

| | |
|---|---|
| | *Vial 1 OPA-Reagenz |
| | *Vial 2 water |
| | *Vial 3 55 Vol.-% n-propanol in water |
| Retention | aspartic acid 8.6 min; homoserine 9.1 min; threonine 9.8 |
| times | min; glycine |
| | 10.1 min; acetylhomoserine 11.6 min; methionine 13.3 min; |
| | valine |
| | 13.8 min; isoleucine 14.7 min; lysine 15.1 min |

Implementation of μ-GC Online Measurements of Ethane, Oxygen, Nitrogen, and, Carbon Dioxide and Determination of Transfer Rates and Connection of Fermenters to the μ-GC

All fermenters were equipped with sterile filters (0.22 μm) with NPT-thread to ensure tightness of the off-gas stream and enable mass balancing. Behind the sterile filters, a tee was installed with the main off-gas stream to the fume hood and a side branch for GC measurements. The side branch ( 1/16″ stainless steel tubing) was connected to a 16 port VICI-valve that is directly connected to the GC. The 16-port valve is controlled by the GC-software. In the μ-GC, a sampling pump is integrated which takes actively samples from the off-gas stream. To make sure, the sample represents the actual fermenter gas composition, the sampling time is 30 s at a flow rate of 9 mL/min to flush the whole sampling line. A second tee is installed in the gas supply of fermenter/unit No1 and No5 to be able to measure the actual gas inlet as a representative for all fermenters (For fermenters 1-4 and 5-8 respectively).

Calibration

For the calibration of the μ-GC, three test gas mixtures were used with a composition of ethane/CO2/N2/O2 of 1: 25/10/50.7/13.65; 2: 30/5/50.7/13.65; 3:35/1/49.92/13.44. Mixture 2 is used as quality control; mixtures 1 and 3 are used for a two point linear calibration. A quality control with mixture 2 is carried out every 30 days. The calibration is done at the installation of the μ-GC, every time, the method is changed, and when the quality control is out of the specification.

GC-Parameters

The μ-GC is equipped with four modules containing four different columns which can be analysed independently by four thermal conductivity detectors (TCD). All four columns are heated in a common oven to 80° C. Column No 1 is a 10 m mol sieve 5 Å (MS5A) with a heated injector (110° C.). To avoid deterioration of the column by water and other contaminants, a backflush of 10 s is set. The column runs at 170 kPa static pressure mode with argon as carrier gas. Column No 1 is used to analyse permanent gases such as oxygen (29.0 s retention time), and nitrogen (30.8 s retention time) with a total runtime of 180 s. With argon as carrier gas, the signal has to be inverted and an approximately two times reduced sensitivity for nitrogen and oxygen is observed compared to helium as carrier gas. Column No 2 is a 10 m PPU column. The backflush is 16 s, the injector temperature 110° C. and the pressure is kept at 150 kPa in static pressure mode with a total runtime of 180 s. On column No 2, carbon dioxide and ethane are analysed with retention times of 31.6 s and 34.5 s respectively. On column No3 and No4, higher molecules can be analysed, for the actual analytical task, they are not necessary.

Online Ethane, Oxygen, and Carbon Dioxide Measurements Using Nitrogen as Internal Standard

For the gassing of the fermenters, either pressurised air or a gas mixing unit (pressurised air plus pure ethane). While passing the fermentation broth the gas composition is changed by oxygen, and ethane consumption, carbon dioxide formation and dilution by saturation with steam. In the case of diluted liquid samples, the consumption of only one analyte does not influence the concentration of the other analytes as there is nearly no change in the total volume. For non-diluted gaseous samples, with all analytes present in significant amounts, the consumption or formation of one analyte drastically influences the concentration (vol.-%) of the other analytes. Therefore, an internal standard is needed. In the actual gas composition, nitrogen is used as an internal standard, as it is neither consumed nor produced during biotransformation and almost insoluble in water. Thus, the dilution factor F_{dil} respectively the change in the gas flow rate inlet vs. outlet is calculated using the respective nitrogen concentrations:

$${F}_{dil} = \frac{{N}_{2 , i n}}{{N}_{2 , out}}$$

With:
N_{2, in}=volume fraction nitrogen inlet
N_{2, out}=volume fraction nitrogen concentration outlet in %

The actual ethane consumption └Vₑₜₕₐₙₑ is then calculated from the difference in ethane volume fraction in the inlet—outlet taking the dilution factor F_{dil} into account:

$$Δ {V}_{ethane} = \dot{V} \cdot ( \frac{{x}_{ethane , i n}}{100} - \frac{{x}_{ethane , out} \cdot {F}_{dil}}{100} )$$

With:
V=total flow rate in L/h
x_{ethane,in}=volume fraction ethane in
x_{ethane,out}=volume fraction ethane out

The calculated ethane volume consumed is converted into the respective amount of ethane [mol] using the ideal gas law.

p·V=n·R·T

With:
p=pressure [Pa]
V=volume [m³]
n=amount of substance [mol]
R=Gas constant=8.3145 J·mol⁻¹·K⁻¹
T=temperature [K]

With these data, the volumetric ethane uptake rate (EUR, mmol*L⁻¹*h⁻¹), oxygen uptake/transfer rate (OUR/OTR, mmol*L⁻¹*h⁻¹) and the carbon dioxide transfer rate (CTR, mmol*L⁻¹*h⁻¹) are determined, as well as the specific EUR in mgₑₜₕₐₙₑ/(g_{CDW}*h).

Results

After 14 h until 40 h process time ethane uptake rate EUR is exceeding 90 mg ethane per g dry weight and hour.

484 mg/L o-Acetyl-L-homoserine were produced in 48.5 h process time with ethane as sole carbon source. Corresponding control strains equipped with expression systems comprising SEQ ID NO:35 and SEQ ID NO:46 did not show any production of o-Acetyl-L-homoserine while both other systems were functional.

### Example 2

Formation of Lysine from Ethane with Escherichia coli.

For the biotransformation of ethane to L-lysine the genetically modified strain E. coli CGSC 12149 lysCfbr_Ec thrAfbr_Ec pACYC184 {PalkS} [alkS_PpGPo1] {PalkB} [bmoXYBZ_Tb PROKKA_02001_Tb PROKKA_02000_Tb bmoC_1_Tb PROKKA_01998_Tb bmoG_Tb] pBR322 {PalkS} [alkS_PpGPo1] {PalkB} [adhA_Cg aldH_Cg] {Placuv5}[metX_Cg]{Ptac}[thrA_fbr_Ec] was used. This strain harbors the following characteristics:
i) Modification of the E. coli CGSC 12149 lysC gene (SEQ ID NO:33), encoding a feedback resistant variant of aspartokinase 3.
ii) Expression of Thauera butanivorans DSM 2080 butane monooxygenase operon (SEQ ID NO:35), comprising of bmoX_Tb (butane monooxygenase hydroxylase BMOH alpha subunit), bmoY_Tb (butane monooxygenase beta subunit), bmoZ_Tb (butane monooxygenase gamma subunit), bmoB_Tb (butane monooxygenase regulatory protein), bmoC_1_Tb (butane monooxygenase reductase), bmoG_Tb (similar to groEL from E. coli) and three putative ORF PROKKA_02001_Tb, PROKKA_02000_Tb and PROKKA_01998_Tb.
iii) Expression of Corynebacterium glutamicum ATCC 13032 adhA_Cg (SEQ ID NO:36), encoding Zn-dependent alcohol dehydrogenases and aldH_Cg (SEQ ID NO:37), encoding NAD-dependent aldehyde dehydrogenases Cgl2796 genes,
iv) Modification and expression of the E. coli W3110 dapA gene (SEQ ID NO:76), encoding a feedback resistant variant of 4-hydroxy-tetrahydrodipicolinate synthase (SEQ ID NO:3) with G84T G250A A251C leading to dapAmod3_Ec.

These characteristics were brought about by:
i. Replacement of E. coli CGSC 12149 lysC gene by another allele of lysC, encoding a feedback resistant variant aspartokinase 3 (point mutation at bp 1055 from C to T (SEQ ID NO:33), T342I (SEQ ID NO:1) with pKO3 derivative 4-47 (SEQ ID NO:40).
ii. Introduction of plasmid pACYC184 {PalkS} [alkS_PpGPo1] {PalkB} [bmoXYBZ_Tb PROKKA_02001_Tb PROKKA_02000_Tb bmoC_1_Tb PROKKA_01998_Tb bmoG_Tb] {PlacUV5} [adhA_Cg aldH_Cg] (SEQ ID NO:41)
iii. Introduction of plasmid pBR322 {PlacUV5} [dapAmod3_Ec] (SEQ ID NO:74)

Construction of pKO3 Modification Vectors

For construction of pKO3 derivatives for gene deletion and/or allelic replacement homologous sequences up- and downstream of the target genes were amplified by PCR from genomic DNA of E. coli W3110 using the primers of SEQ ID NOs: 43-46. Homologous ends for assembly cloning were introduced within the primers. The PCR was performed with Phusion® High-Fidelity Master Mix according to the manufacturer (New England Biolabs, Ipswitch, Mass., USA). The thermal cycle profile was 3 min at 98° C. for initial denaturation, 35 cycles: 10 sec at 98° C., 30 sec at 60° C. to 68° C. (gradient), 20 sec at 72° C. and a final 10 min hold step at 72° C. Purification of PCR products was performed by gel extraction or PCR purification according to the manufacturer of purification kits (QiaQuick PCR Purification Kit and QiaQuick Gel Extraction Kit, Qiagen, Hilden, Germany). Purified PCR products were assembled into NotI restricted pKO3 plasmid using NEBuilder® HiFi DNA Assembly Master Mix according to the manufacturers manual (New England Biolabs, Ipswitch, Mass., USA). Transformation of E. coli DH10β was performed according to the manufacturer (New England Biolabs, Ipswitch, Mass., USA). The final plasmids were verified by restriction analysis and DNA sequencing.

Construction of HM-p-48

Plasmid HM-p-54 (SEQ ID NO:74) is based on plasmid HM-p-25 (SEQ ID NO:41) comprising butane monooxygenase operon of Thauera butanivorans DSM 2080 (SEQ ID NO. 3), comprising of bmoX_Tb (butane monooxygenase hydroxylase BMOH alpha subunit), bmoY_Tb (butane monooxygenase beta subunit), bmoZ_Tb (butane monooxygenase gamma subunit), bmoB_Tb (butane monooxygenase regulatory protein), bmoC_1_Tb (butane monooxygenase reductase), bmoG_Tb (similar to groEL from E. coli) and three putative ORF PROKKA_02001_Tb, PROKKA_02000_Tb and PROKKA_01998_Tb. Additionally, gene expression of C. glutamicum ATCC 13032 adhA_Cg (SEQ ID NO:36), encoding Zn-dependent alcohol dehydrogenases and aldH_Cg (SEQ ID NO:37), encoding NAD-dependent aldehyde dehydrogenases Cgl2796 was enabled by amplifying genes by PCR from AP-p-125 (SEQ ID NO:69) including lacUVS pro motor region (SEQ ID NO:77). Homologous ends for assembly cloning were introduced within the primers. The final plasmid was verified by restriction analysis and DNA sequencing (SEQ ID NO:75).

Construction of HM-p-54

For expression of the E. coli W3110 dapA gene (DNA: SEQ ID NO:76; Protein: SEQ ID NO:3), encoding a feedback resistant variant of 4-hydroxy-tetrahydrodipicolinate synthase with G84T G250A A251C leading to dapAmod3_Ec was ordered as synthetic gene construct (SEQ ID NO:76). This synthetic gene was fused to a lacUVS promotor by in vitro recombination and cloned into pBR322 base vector. The final plasmid was verified by restriction analysis and sequencing (SEQ ID NO:74).

Construction of Strain GAO-EC-149

E. coli CGSC 12149 wild type was modified according to pKO3 procedure (Link A J, Phillips D, Church G M. J Bateriol. 179(20):6228-37) with plasmid according to SEQ ID NO:40. Modifications lead to E. coli CGSC 12149 lysCfbr_Ec. This strain was transformed with plasmids according to SEQ ID NO:74 and SEQ ID NO:75. Transformation of E. coli derivatives was performed via electroporation as known in the art. This work resulted in E. coli strain GAO-EC-149.

DASGIP Testing GAO-EC-149

Materials, methods and analytics are the same as Example 1.

Results

After 14 h until 40 h process time ethane uptake rate EUR exceeding 60 mg ethane per g dry weight and hour.

1211 mg/L L-lysine in 48.5 h process time were produced, thereby half was produced while glucose feed was still running (14 h process time), remaining 480 mg/L L-lysine was produced with ethane as sole carbon source. Corresponding control strains equipped with expression systems comprising SEQ ID NO:35 and SEQ ID NO:46 did not show any production of L-lysine while both other systems were functional.

### Example 3

As listed in table 1 different amino acids are produced by various bacteria with increased expression of the specific enzymes as referenced in the tables. An alkane mixture comprising ethane, propane and butane at a weight ratio of 1:1:1 is used as alkane. All enzyme entries are NCBI accession numbers. For the enzymes E6 of the type E6a, E6c, E6e, E6k, E6I and E6s also feedback-insensitive variants of the sequences indicated may be used.

| | | | | | | E6, in [ ] type | Amino acid to be |
|---|---|---|---|---|---|---|---|
| # | Host cell | E1 | E2 | E3 | E4 | of E6 | produced |
| 1 | E. coli | AAM19727.1 and | BAA36121.1 | BAA36121.1 | None, or | BAE77370.1 | Threonine |
| | | AAM19728.1 and | | | P27550.2, or | [a] | |
| | | AAM19729.1 and | | | APC52536.1, | | |
| | | AAM19730.1 and | | | and | | |
| | | AAM19731.1 and | | | P0A9M8.2, or | | |
| | | AAM19732.1 and | | | BAA16336.1 | | |
| | | ABU68845.2 | | | | | |
| 1 | E. coli | AAM19727.1 and | WP_011015397.1 | WP_011015386.1 | None, or | BAE77859.1 | Threonine |
| | | AAM19728.1 and | | | P27550.2, or | [b] | |
| | | AAM19729.1 and | | | APC52536.1, | | |
| | | AAM19730.1 and | | | and | | |
| | | AAM19731.1 and | | | P0A9M8.2, or | | |
| | | AAM19732.1 and | | | BAA16336.1 | | |
| | | ABU68845.2 | | | | | |
| 1 | E. coli | AAM19727.1 and | NP_745969.1 | NP_742708.1 | None, or | TLD77709.1 | Threonine |
| | | AAM19728.1 and | | | P27550.2, or | [n] | |
| | | AAM19729.1 and | | | APC52536.1, | | |
| | | AAM19730.1 and | | | and | | |
| | | AAM19731.1 and | | | P0A9M8.2, or | | |
| | | AAM19732.1 and | | | BAA16336.1 | | |
| | | ABU68845.2 | | | | | |
| 2 | E. coli | AAM19727.1 and | BAA36121.1 | NP_744824.1 | None, or | APC53474.1 | O- |
| | | AAM19728.1 and | | | P27550.2, or | [g] | Acetylhomoserine |
| | | AAM19729.1 and | | | APC52536.1, | | |
| | | AAM19730.1 and | | | and | | |
| | | AAM19731.1 and | | | P0A9M8.2, or | | |
| | | AAM19732.1 and | | | BAA16336.1 | | |
| | | ABU68845.2 | | | | | |
| 2 | E. coli | AAM19727.1 and | WP_011015397.1 | WP_011015386.1 | None, or | APC51865.1 | O- |
| | | AAM19728.1 and | | | P27550.2, or | [h] | Acetylhomoserine |
| | | AAM19729.1 and | | | APC52536.1, | | |
| | | AAM19730.1 and | | | and | | |
| | | AAM19731.1 and | | | P0A9M8.2, or | | |
| | | AAM19732.1 and | | | BAA16336.1 | | |
| | | ABU68845.2 | | | | | |
| 2 | E. coli | AAM19727.1 and | NP_745969.1 | NP_742708.1 | None, or | NP_747448.1 | O- |
| | | AAM19728.1 and | | | P27550.2, or | [i] | Acetylhomoserine |
| | | AAM19729.1 and | | | APC52536.1, | | |
| | | AAM19730.1 and | | | and | | |
| | | AAM19731.1 and | | | P0A9M8.2, or | | |
| | | AAM19732.1 and | | | BAA16336.1 | | |
| | | ABU68845.2 | | | | | |
| 3 | E. coli | AAM19727.1 and | BAA36121.1 | NP_744824.1 | None, or | BAB96580.2 | Methionine |
| | | AAM19728.1 and | | | P27550.2, or | [l] | |
| | | AAM19729.1 and | | | APC52536.1, | | |
| | | AAM19730.1 and | | | and | | |
| | | AAM19731.1 and | | | P0A9M8.2, or | | |
| | | AAM19732.1 and | | | BAA16336.1 | | |
| | | ABU68845.2 | | | | | |
| 3 | E. coli | AAM19727.1 and | WP_011015397.1 | WP_011015386.1 | None, or | BAB96579.2 | Methionine |
| | | AAM19728.1 and | | | P27550.2, or | [k] | |
| | | AAM19729.1 and | | | APC52536.1, | | |
| | | AAM19730.1 and | | | and | | |
| | | AAM19731.1 and | | | P0A9M8.2, or | | |
| | | AAM19732.1 and | | | BAA16336.1 | | |
| | | ABU68845.2 | | | | | |
| 3 | E. coli | AAM19727.1 and | BAA36121.1 | BAA36121.1 | None, or | APC51864.1 | Methionine |
| | | AAM19728.1 and | | | P27550.2, or | [h] | |
| | | AAM19729.1 and | | | APC52536.1, | | |
| | | AAM19730.1 and | | | and | | |
| | | AAM19731.1 and | | | P0A9M8.2, or | | |
| | | AAM19732.1 and | | | BAA16336.1 | | |
| | | ABU68845.2 | | | | | |
| 4 | E. coli | AAM19727.1 and | WP_011015397.1 | WP_011015386.1 | None, or | BAA16355.1 | Lysine |
| | | AAM19728.1 and | | | P27550.2, or | [c] | |
| | | AAM19729.1 and | | | APC52536.1, | | |
| | | AAM19730.1 and | | | and | | |
| | | AAM19731.1 and | | | P0A9M8.2, or | | |
| | | AAM19732.1 and | | | BAA16336.1 | | |
| | | ABU68845.2 | | | | | |
| 4 | E. coli | AAM19727.1 and | NP_745969.1 | NP_742708.1 | None, or | CAF19965.1 | Lysine |
| | | AAM19728.1 and | | | P27550.2, or | [f] | |
| | | AAM19729.1 and | | | APC52536.1, | | |
| | | AAM19730.1 and | | | and | | |
| | | AAM19731.1 and | | | P0A9M8.2, or | | |
| | | AAM19732.1 and | | | BAA16336.1 | | |
| | | ABU68845.2 | | | | | |
| 4 | E. coli | AAM19727.1 and | BAA36121.1 | NP_744824.1 | None, or | BAB96600.1 | Lysine |
| | | AAM19728.1 and | | | P27550.2, or | [d] | |
| | | AAM19729.1 and | | | APC52536.1, | | |
| | | AAM19730.1 and | | | and | | |
| | | AAM19731.1 and | | | P0A9M8.2, or | | |
| | | AAM19732.1 and | | | BAA16336.1 | | |
| | | ABU68845.2 | | | | | |
| 5 | C. glutamicum | AAM19727.1 and | WP_011015397.1 | WP_011015386.1 | None, or | BAB97645.1 | Threonine |
| | | AAM19728.1 and | | | BAC00146.1, | [a] | |
| | | AAM19729.1 and | | | and | | |
| | | AAM19730.1 and | | | BAC00147.1, | | |
| | | AAM19731.1 and | | | or P27550.2 | | |
| | | AAM19732.1 and | | | | | |
| | | ABU68845.2 | | | | | |
| 5 | C. glutamicum | AAM19727.1 and | NP_745969.1 | NP_742708.1 | None, or | CAF19888.1 | Threonine |
| | | AAM19728.1 and | | | BAC00146.1, | [l] | |
| | | AAM19729.1 and | | | and | | |
| | | AAM19730.1 and | | | BAC00147.1, | | |
| | | AAM19731.1 and | | | or P27550.2 | | |
| | | AAM19732.1 and | | | | | |
| | | ABU68845.2 | | | | | |
| 5 | C. glutamicum | AAM19727.1 and | WP_011015397.1 | WP_011015386.1 | None, or | BAB98978.1 | Threonine |
| | | AAM19728.1 and | | | BAC00146.1, | [g] | |
| | | AAM19729.1 and | | | and | | |
| | | AAM19730.1 and | | | BAC00147.1, | | |
| | | AAM19731.1 and | | | or P27550.2 | | |
| | | AAM19732.1 and | | | | | |
| | | ABU68845.2 | | | | | |
| 6 | C. glutamicum | AAM19727.1 and | NP_745969.1 | NP_742708.1 | None, or | BAB98576.1 | O- |
| | | AAM19728.1 and | | | BAC00146.1, | [k] | Acetylhomoserine |
| | | AAM19729.1 and | | | and | | |
| | | AAM19730.1 and | | | BAC00147.1, | | |
| | | AAM19731.1 and | | | or P27550.2 | | |
| | | AAM19732.1 and | | | | | |
| | | ABU68845.2 | | | | | |
| 6 | C. glutamicum | AAM19727.1 and | WP_011015397.1 | WP_011015386.1 | None, or | NP_747447.1 | O- |
| | | AAM19728.1 and | | | BAC00146.1, | [i] | Acetylhomoserine |
| | | AAM19729.1 and | | | and | | |
| | | AAM19730.1 and | | | BAC00147.1, | | |
| | | AAM19731.1 and | | | or P27550.2 | | |
| | | AAM19732.1 and | | | | | |
| | | ABU68845.2 | | | | | |
| 6 | C. glutamicum | AAM19727.1 and | NP_745969.1 | NP_742708.1 | None, or | BAB98082.1 | O- |
| | | AAM19728.1 and | | | BAC00146.1, | [i] | Acetylhomoserine |
| | | AAM19729.1 and | | | and | | |
| | | AAM19730.1 and | | | BAC00147.1, | | |
| | | AAM19731.1 and | | | or P27550.2 | | |
| | | AAM19732.1 and | | | | | |
| | | ABU68845.2 | | | | | |
| 7 | C. glutamicum | AAM19727.1 and | NP_745969.1 | NP_742708.1 | None, or | NP_599504.1 | Methionine |
| | | AAM19728.1 and | | | BAC00146.1, | [a] | |
| | | AAM19729.1 and | | | and | | |
| | | AAM19730.1 and | | | BAC00147.1, | | |
| | | AAM19731.1 and | | | or P27550.2 | | |
| | | AAM19732.1 and | | | | | |
| | | ABU68845.2 | | | | | |
| 7 | C. glutamicum | AAM19727.1 and | WP_011015397.1 | WP_011015386.1 | None, or | CAF19359.1 | Methionine |
| | | AAM19728.1 and | | | BAC00146.1, | [o] | |
| | | AAM19729.1 and | | | and | | |
| | | AAM19730.1 and | | | BAC00147.1, | | |
| | | AAM19731.1 and | | | or P27550.2 | | |
| | | AAM19732.1 and | | | | | |
| | | ABU68845.2 | | | | | |
| 7 | C. glutamicum | AAM19727.1 and | NP_745969.1 | NP_742708.1 | None, or | CAF21108.1 | Methionine |
| | | AAM19728.1 and | | | BAC00146.1, | [u] | |
| | | AAM19729.1 and | | | and | | |
| | | AAM19730.1 and | | | BAC00147.1, | | |
| | | AAM19731.1 and | | | or P27550.2 | | |
| | | AAM19732.1 and | | | | | |
| | | ABU68845.2 | | | | | |
| 8 | C. glutamicum | AAM19727.1 and | BAA36121.1 | BAA36121.1 | None, or | CAF20314.1 | Lysine |
| | | AAM19728.1 and | | | BAC00146.1, | [d] | |
| | | AAM19729.1 and | | | and | | |
| | | AAM19730.1 and | | | BAC00147.1, | | |
| | | AAM19731.1 and | | | or P27550.2 | | |
| | | AAM19732.1 and | | | | | |
| | | ABU68845.2 | | | | | |
| 8 | C. glutamicum | AAM19727.1 and | WP_011015397.1 | WP_011015386.1 | None, or | CAF19884.1 | Lysine |
| | | AAM19728.1 and | | | BAC00146.1, | [e] | |
| | | AAM19729.1 and | | | and | | |
| | | AAM19730.1 and | | | BAC00147.1, | | |
| | | AAM19731.1 and | | | or P27550.2 | | |
| | | AAM19732.1 and | | | | | |
| | | ABU68845.2 | | | | | |
| 8 | C. glutamicum | AAM19727.1 and | NP_745969.1 | NP_742708.1 | None, or | P75826.2 | Lysine |
| | | AAM19728.1 and | | | BAC00146.1, | [f] | |
| | | AAM19729.1 and | | | and | | |
| | | AAM19730.1 and | | | BAC00147.1, | | |
| | | AAM19731.1 and | | | or P27550.2 | | |
| | | AAM19732.1 and | | | | | |
| | | ABU68845.2 | | | | | |
| 8 | C. glutamicum | AAM19727.1 and | BAA36121.1 | BAA36121.1 | None, or | BAE76111.1 | Lysine |
| | | AAM19728.1 and | | | BAC00146.1, | [f] | |
| | | AAM19729.1 and | | | and | | |
| | | AAM19730.1 and | | | BAC00147.1, | | |
| | | AAM19731.1 and | | | or P27550.2 | | |
| | | AAM19732.1 and | | | | | |
| | | ABU68845.2 | | | | | |
| 8 | C. glutamicum | AAM19727.1 and | WP_011015397.1 | WP_011015386.1 | None, or | CAF19884.1 | Lysine |
| | | AAM19728.1 and | | | BAC00146.1, | [e] | |
| | | AAM19729.1 and | | | and | | |
| | | AAM19730.1 and | | | BAC00147.1, | | |
| | | AAM19731.1 and | | | or P27550.2 | | |
| | | AAM19732.1 and | | | | | |
| | | ABU68845.2 | | | | | |
| 9 | P. putida | AAM19727.1 and | NP_745969.1 | NP_742708.1 | None, or | NP_746584.1 | Threonine |
| | | AAM19728.1 and | | | APC52536.1, | [a] | |
| | | AAM19729.1 and | | | and | | |
| | | AAM19730.1 and | | | P0A9M8.2, or | | |
| | | AAM19731.1 and | | | NP_746598.2, | | |
| | | AAM19732.1 and | | | or | | |
| | | ABU68845.2 | | | NP_746811.1 | | |
| 9 | P. putida | AAM19727.1 and | NP_745969.1 | NP_742708.1 | None, or | NP_747448.1 | Threonine |
| | | AAM19728.1 and | | | APC52536.1, | [l] | |
| | | AAM19729.1 and | | | and | | |
| | | AAM19730.1 and | | | P0A9M8.2, or | | |
| | | AAM19731.1 and | | | NP_746598.2, | | |
| | | AAM19732.1 and | | | or | | |
| | | ABU68845.2 | | | NP_746811.1 | | |
| 9 | P. putida | AAM19727.1 and | BAA36121.1 | BAA36121.1 | None, or | NP_744388.1 | Threonine |
| | | AAM19728.1 and | | | APC52536.1, | [n] | |
| | | AAM19729.1 and | | | and | | |
| | | AAM19730.1 and | | | P0A9M8.2, or | | |
| | | AAM19731.1 and | | | NP_746598.2, | | |
| | | AAM19732.1 and | | | or | | |
| | | ABU68845.2 | | | NP_746811.1 | | |
| 10 | P. putida | AAM19727.1 and | BAA36121.1 | BAA36121.1 | wildtype | NP_743662.1 | O- |
| | | AAM19728.1 and | | | | [k] | Acetylhomoserine |
| | | AAM19729.1 and | | | | | |
| | | AAM19730.1 and | | | | | |
| | | AAM19731.1 and | | | | | |
| | | AAM19732.1 and | | | | | |
| | | ABU68845.2 | | | | | |
| 10 | P. putida | AAM19727.1 and | WP_011015397.1 | WP_011015386.1 | wildtype | BAA35485.1 | O- |
| | | AAM19728.1 and | | | | [ad] | Acetylhomoserine |
| | | AAM19729.1 and | | | | | |
| | | AAM19730.1 and | | | | | |
| | | AAM19731.1 and | | | | | |
| | | AAM19732.1 and | | | | | |
| | | ABU68845.2 | | | | | |
| 10 | P. putida | AAM19727.1 and | NP_745969.1 | NP_742708.1 | wildtype | NP_747198.1 | O- |
| | | AAM19728.1 and | | | | [s] | Acetylhomoserine |
| | | AAM19729.1 and | | | | | |
| | | AAM19730.1 and | | | | | |
| | | AAM19731.1 and | | | | | |
| | | AAM19732.1 and | | | | | |
| | | ABU68845.2 | | | | | |
| 11 | P. putida | AAM19727.1 and | BAA36121.1 | NP_744824.1 | wildtype | NP_744143.1 | Methionine |
| | | AAM19728.1 and | | | | [b] | |
| | | AAM19729.1 and | | | | | |
| | | AAM19730.1 and | | | | | |
| | | AAM19731.1 and | | | | | |
| | | AAM19732.1 and | | | | | |
| | | ABU68845.2 | | | | | |
| 11 | P. putida | AAM19727.1 and | WP_011015397.1 | WP_011015386.1 | wildtype | NP_742819.1 | Methionine |
| | | AAM19728.1 and | | | | [q] | |
| | | AAM19729.1 and | | | | | |
| | | AAM19730.1 and | | | | | |
| | | AAM19731.1 and | | | | | |
| | | AAM19732.1 and | | | | | |
| | | ABU68845.2 | | | | | |
| 11 | P. putida | AAM19727.1 and | NP_745969.1 | NP_742708.1 | wildtype | BAE78143.1 | Methionine |
| | | AAM19728.1 and | | | | [t] | |
| | | AAM19729.1 and | | | | | |
| | | AAM19730.1 and | | | | | |
| | | AAM19731.1 and | | | | | |
| | | AAM19732.1 and | | | | | |
| | | ABU68845.2 | | | | | |
| 12 | P. putida | AAM19727.1 and | WP_011015397.1 | WP_011015386.1 | wildtype | NP_744186.1 | Lysine |
| | | AAM19728.1 and | | | | [c] | |
| | | AAM19729.1 and | | | | | |
| | | AAM19730.1 and | | | | | |
| | | AAM19731.1 and | | | | | |
| | | AAM19732.1 and | | | | | |
| | | ABU68845.2 | | | | | |
| 12 | P. putida | AAM19727.1 and | NP_745969.1 | NP_742708.1 | wildtype | NP_747328.1 | Lysine |
| | | AAM19728.1 and | | | | [e] | |
| | | AAM19729.1 and | | | | | |
| | | AAM19730.1 and | | | | | |
| | | AAM19731.1 and | | | | | |
| | | AAM19732.1 and | | | | | |
| | | ABU68845.2 | | | | | |
| 12 | P. putida | AAM19727.1 and | BAA36121.1 | NP_744824.1 | wildtype | NP_746833.1 | Lysine |
| | | AAM19728.1 and | | | | [d] | |
| | | AAM19729.1 and | | | | | |
| | | AAM19730.1 and | | | | | |
| | | AAM19731.1 and | | | | | |
| | | AAM19732.1 and | | | | | |
| | | ABU68845.2 | | | | | |

1: A microbial cell for producing at least one L-amino acid from at least one C1-C4 alkane, wherein the cell comprises:
(i) an increased expression relative to the wild type cell of Enzyme E₁ capable of converting the alkane to a corresponding 1-alkanol;
(ii) an increased expression relative to the wild type cell of Enzyme E₂ capable of converting the 1-alkanol of (i) to a corresponding aldehyde; and either
(in) (A)
an increased expression relative to the wild type cell of Enzyme E₃ capable of converting the aldehyde of (ii) to a corresponding alkanoic acid; and
a wild-type level expression of Enzyme E₄ or an increased expression relative to the wild type cell of Enzyme E₄ capable of converting the alkanoic acid of (iii) to a corresponding fatty acyl thioester; or
(B)
an increased expression relative to the wild type cell of Enzyme E₅ capable of converting the aldehyde of (ii) to a corresponding fatty acyl thioester;
and
(iv) an increased expression relative to the wild type cell of Enzyme E₆ capable of converting the fatty acyl thioester of (iii) to a corresponding amino acid.

2: The cell according to claim 1, wherein the amino acid produced is selected from the group consisting of lysine, threonine, and O-acetyl homoserine.

3: The cell according to claim 1, wherein the amino acid produced is lysine and
the Enzyme E₁ is selected from the group consisting of P450 alkane hydroxylase (Eₐ) of EC 1.14.15.3-, AlkB alkane hydroxylase (E₆) of EC 1.14.15.3 from the AlkBGT component, methane monooxygenase (Eₖ) of EC 1.14.18.3, 1.14.99.39 or 1.14.13.25, propane monooxygenase (Eₗ) of EC. 1.14.13.227 and butane monooxygenase (Eₘ) of EC 1.14.13.230;
the Enzyme E₂ is selected from the group consisting of P450 alkane hydroxylase (Eₐ) of EC 1.14.15.3-, AlkB alkane hydroxylase (E_{b}) of EC 1.14.15.3 from the AlkBGT component, alcohol oxidase (E_{c}) of EC 1.1.3.20 and alcohol dehydrogenase (E_{d}) of EC 1.1.1.1 or EC 1.1.1.2;
the Enzyme E₃ is selected from the group consisting of P450 alkane hydroxylase (Eₐ) of EC 1.14.15.3-, AlkB alkane hydroxylase (E_{b}) of EC 1.14.15.3 from the AlkBGT component, aldehyde dehydrogenase (Eₑ) of EC 1.2.1.3, EC 1.2.1.4 or EC 1.2.1.5, bifunctional alcohol oxidase (E_{c}) of EC 1.1.3.20, bifunctional AlkJ alcohol dehydrogenase (E_{di}) of EC 1.1.99 and bifunctional alcohol dehydrogenase (E_{dii}) of EC 1.1.1.1 or EC 1.1.1.2, wherein E_{c}, E_{di}, and E_{dii} are capable of oxidizing an ω-hydroxy alkanoic acid ester directly to the corresponding ω-carboxy alkanoic acid ester;
the Enzyme E₄ is selected from the group consisting of fatty acyl coenzyme A (CoA) synthase (E_{f}) of EC 6.2.1.1, EC 6.2.1.2 or EC 6.2.1.3, acyl-Acyl Carrier Protein (ACP) synthase (E_{g}) of EC 6.2.1.20 or EC 6.2.1.47, a combination of fatty acyl kinase (Eₕ) of EC 2.7.2.1, EC 2.7.2.12, EC 2.7.2.15 or EC 2.7.2.7 and phosphotransacylase (Eᵢ) of EC 2.3.1.8 or EC 2.3.1.19, and a combination of fatty acyl-CoA synthase (E_{f}) of EC 6.2.1.1, EC 6.2.1.2 or EC 6.2.1.3 and a fatty acyl-CoA:ACP transacylase (Eⱼ) of EC 2.3.1.38 or EC 2.3.1.39;
the Enzyme E₅ is an CoA-linked aldehyde dehydrogenase (Eₑᵢ) of EC 1.2.1.10 or EC 1.2.1.87; and
the Enzyme E₆ is selected from the group consisting of aspartate kinase (E₆ₐ) (EC 2.7.2.4), aspartate semialdehyde dehydrogenase (E_{6b}) (EC 1.2.1.114-hydroxy-tetrahydrodipicolinate synthase (E_{6c}) (EC 1.4.1.16), dihydrodipicolinate reductase (E_{6d}) (EC 1.17.1.8), diaminopimelate decarboxylase (E₆ₑ) (EC 4.1.1.20), lysine exporter (E_{6f}) (TCDB families 2.A.124.1.1, 2.A.75.1.1 or 2.A.75.1.2), phosphoenolpyruvate (PEP) carboxylase (E_{6g}) (EC 4.1.1.31), proton-translocating transhydrogenase (E₆ₕ) (EC 1.6.1.5), and pyruvate carboxylase (E₆ᵢ) (EC 6.4.1.1).

4: The cell according to claim 3, wherein the enzyme E₆ is selected from the group consisting of aspartate kinase (E₆ₐ) and 4-hydroxy-tetrahydrodipicolinate synthase (E_{6c}) (EC 1.4.1.16).

5: The cell according to claim 3, wherein the enzyme E₆ is a feedback resistant variant of aspartate kinase (E₆ₐ) comprising SEQ ID NOT, or a feedback resistant variant of 4-hydroxy-tetrahydrodipicolinate synthase (E_{6c}) (EC 1.4.1.16) comprising SEQ ID NOT.

6: The cell according to claim 1, wherein the amino acid produced is lysine and the enzyme
E₁ is a butane monoxygenase (E_{c}) (EC 1.14.13.230);
E₂ is an alcohol dehydrogenase (E_{d}) (EC 1.1.1.1 or EC 1.1.1.2);
E₃ is an aldehyde dehydrogenase (Eₑ) (EC 1.2.1.3, EC 1.2.1.4 or EC 1.2.1.5);
E₄ is fatty acyl CoA synthase (FACS) (E_{f}) (EC 6.2.1.1, EC 6.2.1.2 or EC 6.2.1.3);
E₆ is at least one enzyme selected from the group consisting of:
(i) a feedback-resistant variant of aspartate kinase (E₆ₐ) and
(ii) a feedback-resistant variant of 4-hydroxy-tetrahydrodipicolinate synthase (E_{6c}) (EC 1.4.1.16).

7: The cell according to claim 1, wherein the amino acid produced is O-acetyl homoserine and
the Enzyme E₁ is selected from the group consisting of P450 alkane hydroxylase (Eₐ) of EC 1.14.15.3-, AlkB alkane hydroxylase (E_{b}) of EC 1.14.15.3 from the AlkBGT component, methane monooxygenase (Eₖ) of EC 1.14.18.3, 1.14.99.39 or 1.14.13.25, propane monooxygenase (Eₗ) of EC. 1.14.13.227 and butane monooxygenase (Eₘ) of EC 1.14.13.230;
the Enzyme E₂ is selected from the group consisting of P450 alkane hydroxylase (Eₐ) of EC 1.14.15.3-, AlkB alkane hydroxylase (E_{b}) of EC 1.14.15.3 from the AlkBGT component, alcohol oxidase (E_{c}) of EC 1.1.3.20 and alcohol dehydrogenase (E_{d}) of EC 1.1.1.1 or EC 1.1.1.2;
the Enzyme E₃ is selected from the group consisting of P450 alkane hydroxylase (Eₐ) of EC 1.14.15.3-, AlkB alkane hydroxylase (E_{b}) of EC 1.14.15.3 from the AlkBGT component, aldehyde dehydrogenase (Eₑ) of EC 1.2.1.3, EC 1.2.1.4 or EC 1.2.1.5, bifunctional alcohol oxidase (E_{c}) of EC 1.1.3.20, bifunctional AlkJ alcohol dehydrogenase (E_{di}) of EC 1.1.99 and bifunctional alcohol dehydrogenase (E_{dii}) of EC 1.1.1.1 or EC 1.1.1.2, wherein E_{c}, E_{di}, and E_{dii} are capable of oxidizing an ω-hydroxy alkanoic acid ester directly to the corresponding ω-carboxy alkanoic acid ester;
the Enzyme E₄ is selected from the group consisting of fatty acyl coenzyme A (CoA) synthase (E_{f}) of EC 6.2.1.1, EC 6.2.1.2 or EC 6.2.1.3, acyl-Acyl Carrier Protein (ACP) synthase (E_{g}) of EC 6.2.1.20 or EC 6.2.1.47, a combination of fatty acyl kinase (Eₕ) of EC 2.7.2.1, EC 2.7.2.12, EC 2.7.2.15 or EC 2.7.2.7 and phosphotransacylase (Eᵢ) of EC 2.3.1.8 or EC 2.3.1.19, and a combination of fatty acyl-CoA synthase (E_{f}) of EC 6.2.1.1, EC 6.2.1.2 or EC 6.2.1.3 and a fatty acyl-CoA:ACP transacylase (Eⱼ) of EC 2.3.1.38 or EC 2.3.1.39;
the Enzyme E₅ is an CoA-linked aldehyde dehydrogenase (Eₑᵢ) of EC 1.2.1.10 or EC 1.2.1.87; and
the Enzyme E₆ is selected from the group consisting of aspartate kinase (E₆ₐ) (EC 2.7.2.4), aspartate semi aldehyde dehydrogenase (E_{6b}) (EC 1.2.1.11), glyceraldehyde-3-phosphate dehydrogenase (NADP-dependent) (E₆J) (EC 1.2.1.9, EC 1.2.1.13, EC 1.2.1.59, EC 1.2.1.60), homoserine dehydrogenase (E₆ₖ) (EC 1.1.1.3), homoserine kinase (E₆ₗ) (EC 2.7.1.39), phosphoenolpyruvate (PEP) carboxylase (E_{6g}) (EC 4.1.1.31), proton-translocating transhydrogenase (E₆ₕ) (EC 1.6.1.5), pyruvate carboxylase (E₆ᵢ) (EC 6.4.1.1), homoserine O-acetyltransferase (E₆ₛ) (EC 2.3.1.31), and O-acetyl homoserine exporter (E_{6ad}) (TCDB classification 2.A.42.2.2; 2.A.7.3.6; 2.A.76.1.10; 2.A.76.1.2; 2.A.79.1.1; 2.A.95.1.4, 2.A.7.21.5, 2.A.76.1.1, 2.A.76.1.9).

8: The cell according to claim 7, wherein the enzyme E₆ is selected from the group consisting of aspartate kinase (E₆ₐ), homoserine dehydrogenase (E₆ₖ) and homoserine O-acetyltransferase (E₆ₛ).

9: The cell according to claim 6, wherein the enzyme E₆ is homoserine dehydrogenase (E₆ₖ) comprising SEQ ID NO: 14, 51, 80 or a variant thereof, or a homoserine O-acetyltransferase (E₆ₛ) comprising SEQ ID NO: 16, 78 or a variant thereof.

10: The cell according to claim 1, wherein the amino acid produced is O-acetyl homoserine and the enzyme
E₁ is a butane monoxygenase (E_{c}) (EC 1.14.13.230);
E₂ is an alcohol dehydrogenase (E_{d}) (EC 1.1.1.1 or EC 1.1.1.2);
E₃ is an aldehyde dehydrogenase (Eₑ) (EC 1.2.1.3, EC 1.2.1.4 or EC 1.2.1.5);
E₄ is fatty acyl CoA synthase (FACS) (E_{f}) (EC 6.2.1.1, EC 6.2.1.2 or EC 6.2.1.3); and
E₆ is at least one enzyme selected from the group consisting of:
(i) a feedback resistant variant of homoserine dehydrogenase (E₆ₖ),
(ii) a feedback-resistant variant of aspartate kinase (E₆ₐ) comprising SEQ ID NO:1 with a point mutation of T342I, or SEQ ID NO:79 with at least one point mutation selected from the group consisting of T311I, A279T, S301Y, A279V, S301F, T308I, S317A, R320G, G345D, S381F, Q404E, G408R, G277A, Q298A, T361A, E363A, and F364A, and
(iii) a feedback-resistant variant of homoserine O-acetyltransferase (E₆ₛ) comprising SEQ ID NO:78 with point mutation Y294C.

11: The cell according to claim 1, wherein the cell is selected from the group consisting of Acinetobacter sp., Bacillus sp., Brevibacterium sp., Burkholderia sp., Chlorella sp., Clostridium sp., Corynebacterium sp., Cyanobakterien, Escherichia sp., Pseudomonas sp., Klebsiella sp., Salmonella sp., Rhizobium sp., Saccharomyces sp., Pichia sp., and Nostoc sp.

12: The cell according to claim 1, wherein the cell is selected from the group consisting of Bacillus subtilis, Burkholderia thailandensis, Corynebacterium glutamicum, E. coli, Klebsiella oxytoca, Pseudomonas fluorescens, Pseudomonas putida, Pseudomonas stutzeri, Rhizobium meliloti, Saccharomyces cerevisiae and Pichia pastoris.

13: A method of producing at least one amino acid, wherein the method comprises contacting at least one cell according to claim 1 with at least one C1-C4 alkane.

14: The method according to claim 13, wherein the alkane is ethane or butane and the amino acid is lysine or o-acetyl homoserine.

15. (canceled)
